(19) United States
(12) Patent Application Publication
(10) Pub. No.: US 2025/0011452 A1
(43) Pub. Date: Jan. 9, 2025

(54) DYSFUNCTIONAL ANTIGEN-SPECIFIC CD8+ T CELLS IN THE TUMOR MICROENVIRONMENT

(71) Applicant: The University of Chicago, Chicago, IL (US)

(72) Inventors: Thomas GAJEWSKI, Chicago, IL (US); Jason WILLIAMS, Chicago, IL (US); Brendan HORTON, Chicago, IL (US)

(21) Appl. No.: 18/885,340

(22) Filed: Sep. 13, 2024

Related U.S. Application Data
(63) Continuation of application No. 18/151,112, filed on Jan. 6, 2023, now abandoned.
Application No. 18/151,112 is a continuation of application No. 16/476,219, filed on Jul. 5, 2019, now Pat. No. 11,566,077.
(60) Provisional application No. 62/447,199, filed on Jan. 17, 2017.

(51) Int. Cl. C07K 16/28; A61K 39/00; G01N 33/569

(52) U.S. Cl. CPC C07K 16/2878; A61K 39/4611; A61K 39/4632; A61K 39/464499; A61K 2239/31; A61K 2239/38; C07K 16/2803; G01N 33/56972; C07K 2317/75; C07K 2317/76

(57) ABSTRACT

Provided herein are compositions and methods for detecting and/or targeting dysfunctional tumor antigen-specific CD8⁺ T cells in the tumor microenvironment for diagnostic, therapeutic and/or research applications. In particular, dysfunctional tumor antigen-specific CD8⁺ T cells are detected and/or targeted via their expression of cell surface receptors described herein, such as 4-1BB,LAG-3, or additional markers that correlate with 4-1BB and LAG-3 expression, such as markers differentially expressed on the surface of the T cells.

### CROSS-REFERENCE TO RELATED APPLICATIONS

The present invention is a continuation of U.S. patent application Ser. No. 18/151,112, filed on Jan. 6, 2023, which is a continuation of U.S. patent application Ser. No. 16/476,219, filed on Jul. 5, 2019, now issued as U.S. Pat. No. 11,566,077, issued on Jan. 31, 2023, which is a § 371 National Entry of PCT/US18/14008, filed on Jan. 17, 2018, which claims priority to U.S. Provisional Patent Application No. 62/447,199, filed on Jan. 17, 2017, each of which is incorporated by reference in its entirety.

### SEQUENCE LISTING

The text of the computer readable sequence listing filed herewith, titled “35120-304_SEQUENCE_LISTING”, created Sep. 13, 2024, having a file size of 53,909 bytes, is hereby incorporated by reference in its entirety.

### FIELD

Provided herein are compositions and methods for detecting and/or targeting dysfunctional tumor antigen-specific CD8+ T cells in the tumor microenvironment for diagnostic, therapeutic and/or research applications. In particular, dysfunctional tumor antigen-specific CD8⁺ T cells are detected and/or targeted via their expression of cell surface receptors described herein, such as 4-1BB,LAG-3, or additional markers that correlate with 4-1BB and LAG-3 expression, such as markers differentially expressed on the surface of the T cells.

### BACKGROUND

The immune system plays a critical role in protecting the host from cancer (Vesely et al., 2011; incorporated by reference in its entirety). Innate sensing of tumors leads to an adaptive T cell response through the presentation of tumor-associated antigens (TAAs) derived from mutations and epigenetic changes that contribute to carcinogenesis (Gajewski et al., 2013; incorporated by reference in its entirety). Spontaneously-primed CD8+ T cells home to tumor sites in mouse tumor models (Harlin et al., 2009; Fuertes et al., 2011; incorporated by reference in their entireties) and in a subset of patients with advanced cancer (Harlin et al., 2006; incorporated by reference in its entirety). These tumor-infiltrating lymphocytes (TIL) have the ability to recognize tumor antigens and are believed to contribute to tumor control in cancer patients, based on the correlation between activated CD8+ T cell infiltration with improved prognosis and response to immunotherapy (Fridman et al., 2012; Tumeh et al., 2014; incorporated by reference in their entireties). However, without additional manipulation, this endogenous anti-tumor response is usually not sufficient to mediate complete rejection of an established tumor (Gajewski, 2007b; Pardoll, 2012; Baitsch et al., 2011; Gajewski et al., 2006; Larkin et al., 2015). Data accumulated over the past several years have indicated that tumors with spontaneous anti-tumor T cell responses have high expression of immune-inhibitory pathways that subvert the effector phase of the response. These include PD-L1/PD-1 interactions (Pardoll, 2012; incorporated by reference in its entirety), recruitment of CD4+Foxp3+ regulatory T (Treg) cells (Gajewski, 2007a; incorporated by reference in its entirety), and metabolic dysregulation by indoleamine-2,3-dioxygenase (IDO) (Spranger et al., 2013; incorporated by reference in its entirety). However, even when CD8+ T cells specific for tumor antigens are isolated from tumors, away from these extrinsic immune inhibitory factors, they still show altered functional properties ex vivo (Harlin et al., 2006; Baitsch et al., 2011; incorporated by reference in their entireties).

Expression of PD-1 has been described to identify tumor-specific exhausted T cells (Ahmadzadeh et al., 2009; Fourcade et al., 2012; Wu et al., 2014; Gros et al., 2014; incorporated by reference in their entireties). However, T cells expressing PD-1 in the context of chronic infection can still retain effector function (Wherry and Kurachi, 2015; incorporated by reference in its entirety), and PD-1 is not required for the induction of T cell exhaustion (Odorizzi et al., 2015; incorporated by reference in its entirety). In addition to PD-1, several additional co-inhibitory receptors, including CD223 (LAG-3), CD244 (2B4), T-cell immunoreceptor with Ig and ITIM domains (TIGIT), hepatitis A virus cellular receptor 2 (TIM-3), and cytotoxic T-lymphocyte-associated protein 4 (CTLA-4), are also be expressed on dysfunctional T cells and expression of a greater number of inhibitory receptors has been correlated with diminished cytokine secretion (in particular IFN-g and TNF-a) as well as proliferative capacity (Blackburn et al., 2009; incorporated by reference in its entirety). Expression of these receptors has been observed in both viral and cancer models, however, a complete analysis of both co-inhibitory and co-stimulatory receptors on the same population is lacking in the tumor setting.

### SUMMARY

Provided herein are compositions and methods for detecting and/or targeting dysfunctional tumor antigen-specific CD8+ T cells in the tumor microenvironment for diagnostic, therapeutic and/or research applications. In particular, dysfunctional tumor antigen-specific CD8⁺ T cells are detected and/or targeted via their expression of cell surface receptors described herein, such as 4-1BB,LAG-3, or additional markers that correlate with 4-1BB and LAG-3 expression, such as markers differentially expressed on the surface of the T cells (e.g., PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM and Sema7a).

In some embodiments, provided herein are methods of treating a subject with cancer comprising administering an agent that specifically targets dysfunctional tumor antigen-specific CD8⁺ T cells. In some embodiments, the subject suffers from a solid tumor cancer. In some embodiments, the tumor allows T cell infiltration, but is resistant to immunotherapies. In some embodiments, the tumor environment comprises dysfunctional tumor antigen-specific CD8⁺ T cells. In some embodiments, contacting the dysfunctional tumor antigen-specific CD8⁺ T cells with an anti-4-1BB and/or anti-LAG3 agent. In some embodiments, the anti-4-1BB and/or anti-LAG3 agent is an antibody, antibody fragment, or antibody mimetic molecule. In some embodiments, methods further comprise co-administration of an additional therapeutic agent. In some embodiments, the additional therapeutic agent is a chemotherapeutic or an immunotherapeutic agent. In some embodiments, the additional therapeutic agent is an immunotherapeutic agent selected from the list consisting of cell-based therapies, monoclonal antibody (mAb) therapy, cytokine therapy, and adjuvant treatment. In some embodiments, the immunotherapeutic agent is a mAb therapy selected from the list consisting of anti-CTLA-4 monoclonal antibodies and/or anti-PD-L1 monoclonal antibodies. In some embodiments, the immunotherapeutic agent is a cell-based therapy selected from the list consisting of dendritic-cell therapy and T-cell therapy. In some embodiments, the additional therapeutic agent targets one of the markers/receptors listed in Table 2. In some embodiments, the additional therapeutic targets a marker/receptor expressed on the surface of the T cells. In some embodiments, the additional therapeutic targets PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM or Sema7a. In some embodiments, the additional therapeutic agent targets Nrn1, Sema7a, or CRTAM.

In some embodiments, provided herein are methods of treating a subject with cancer comprising administering a therapeutic agent that specifically targets dysfunctional tumor antigen-specific CD8⁺ T cells, wherein the agent targets one of the receptors listed in Table 2. In some embodiments, the therapeutic targets a marker/receptor expressed on the surface of the T cells. In some embodiments, the therapeutic targets PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM or Sema7a. In some embodiments, the therapeutic agent targets Nrn1, Sema7a, or CRTAM. In some embodiments, the therapeutic agent is an antibody, antibody fragment, or antibody mimetic molecule that binds the target marker/receptor. In some embodiments, the therapeutic agent is an anti-Nm antibody, antibody fragment, or antibody mimetic molecule. In some embodiments, the therapeutic agent is an anti-Sema7a antibody, antibody fragment, or antibody mimetic molecule. In some embodiments, the therapeutic agent is an anti-CRTAM antibody, antibody fragment, or antibody mimetic molecule.

In some embodiments, provided herein are compositions comprising: (a) one or more of an anti-4-1BB agent, an anti-LAG-3 agent, an anti-Nrn1 agent, an anti-Sema7a agent, and an anti-CRTAM agent; and (b) an immunotherapeutic agent, said composition formulated for therapeutic delivery to a subject. In some embodiments, the anti-4-1BB agent, anti-LAG-3 agent, anti-Nrn1 agent, anti-Sema7a agent, and/or anti-CRTAM agent is an antibody, antibody fragment, or antibody mimetic molecule.

In some embodiments, provided herein are compositions comprising: (a) an agent that targets and/or binds one of PD-1, TIM-3, OX-40ICOS, TIGIT, CD244, TNFRSF18, Nrn1, Nrp1, KLRG1, GM156, GPNMB, GPR65, TMEM205, and TMEM126A; and (b) an immunotherapeutic agent, said composition formulated for therapeutic delivery to a subject.

In some embodiments, provided herein are methods comprising: (a) testing CD8⁺ T cells from a cell population to determine whether they co-express LAG-3 and 4-1BB; and (b) administering one or more agents that target and/or bind one of PD-1, TIM-3, OX-40ICOS, TIGIT, CD244, TNFRSF18, Nrn1, Nrp1, KLRG1, GM156, GPNMB, GPR65, TMEM205, and TMEM126A. In some embodiments, the agent is an anti-Nrn1 agent, an anti-Sema7a agent, and an anti-CRTAM agent. In some embodiments, the anti-Nrn1 agent, anti-Sema7a agent, and/or anti-CRTAM agent is an antibody, antibody fragment, or antibody mimetic molecule. In some embodiments, testing is performed in vitro.

In some embodiments, provided herein are methods of identifying dysfunctional T cells by testing said cells for co-expression of 4-1BB and LAG-3. In some embodiments, provided herein are methods of identifying dysfunctional T cells by testing said cells for expression of one or more of the markers/receptors of Table 2 (e.g., a T-cell surface marker/receptor (e.g., PD-1, TIM-3, OX-40ICOS, TIGIT, CD244, TNFRSF18, Nrn1, Nrp1, KLRG1, GM156, GPNMB, GPR65, TMEM205, TMEM126A).

### BRIEF DESCRIPTION OF THE DRAWINGS

FIG. 1A-J. Co-expression of 4-1BB and LAG-3 identifies a significant fraction of the CD8⁺ TIL compartment found in progressing tumors. (A) Representative analysis of 4-1BB and LAG-3 expression on CD8⁺ T cells from B16.SIY tumors and the spleen and TdLN from tumor bearing mice on day 7, 14 and 21 after s.c. tumor inoculation. (B-D) Longitudinal summary of the composition, n=5; four to five independent experiments per time point, (C) absolute cell number, n=5; seven to nine independent experiments per time point, and (D) cellular density of the CD8⁺ 4-1BB/LAG-3 TIL subpopulations, n=5; two to five independent experiments per time point. Absolute cell numbers were determined by acquiring the complete tumor sample by flow cytometery. (E) Day 14 summary of the proportion of the CD8⁺ 4-1BB/LAG-3 TIL subpopulations that are Ki67⁺. n=3-5; two independent experiments. (F) Summary of BrdU uptake on day 13 in the CD8⁺ 4-1BB/LAG-3 TIL subpopulations after a 24 hour BrdU pulse. n=5; three independent experiments. (G-I) Representative flow plots (G and H) and summary (I) of the 4-1BB/LAG-3 populations in other tumor models. Mice were inoculated with 2×10⁶ C1498.SIY, MC38.SIY, EL4.SIY, B16 Parental, MC57.SIY or 1969.SIY subcutaneously and analyzed for 4-1BB and LAG-3 expression on day 14 after tumor inoculation. n=3-5; two to 5 independent experiments for each time point. (J) Mice were inoculated on both flanks with 2×10⁶ MC57.SIY or B16.SIY, at indicated time points tumors from each mouse were pooled and analyzed for co-expression of 4-1BB and LAG-3 in the CD8⁺ TIL compartment. n=3-5; two independent experiments for each time point. All error bars indicate ±SEM. *:P<0.05, **:P<0.01, ***:P<0.001. A two-way ANOVA with Bonferroni post-hoc test was used for (B, C, D, H) longitudinal studies and Kruskal-Wallis (non-parametric) test was used for (E and F) analysis at one time-point.

FIG. 2A-G. Egr2 and a component of the Egr2-transcriptional network are enriched in 4-1BB⁺LAG-3⁺ CD8⁺ TILs. (A) Representative flow plot and summary of Egr2^{EGFP} expression. Egr2^{EGFP} mice were inoculated with 2×10⁶B16.SIY tumors s.c. CD8⁺ T cells from the tumor, TdLN and spleen were analyzed for Egr2^{EGFP} expression on day 7 and day 14. n=4-5; two-independent experiments. (B) Expression of Egr2 target genes (Zheng et al., 2013). CD8⁺ TILs from day 14 tumor bearing mice were sorted based on high or low expression of Egr2^{EGFP} and analyzed directly for expression of Egr2 targets by qRT-PCR. Two tumors on opposite flanks pooled per mouse. n=3; two independent experiments. (C) Representative flow plots and summary of the 4-1BB/LAG-3 subpopulations in CD8⁺ Egr2GFP^{hi} and Egr2GFP^{lo} TILs on day 7 and 14. n=4-5. Two-independent experiments per time point. (D) Expression of Egr2 targets in the 4-1BB⁺LAG-3⁺ and 4-1BB⁻LAG-3⁻ subpopulations. The subpopulations were sorted and analyzed directly for the expression of targets by qRT-PCR. Two tumors on opposite flanks pooled per mouse. n=4; two-independent experiments. (E) Egr2^{flox/flox}×pLCKCreERT2×YFP−Rosa26 mice given 5 doses of tamoxifen by gavage and inoculated 3 days later with 2×10⁶ B16.SIY cells. YFP⁺ or YFP⁻ CD8⁺ TILs were sorted and analyzed for Egr2 transcript directly and after in vitro stimulation. Two tumors on opposite flanks pooled per mouse. n=3; two independent experiments. (F) Representative flow plots and summary of 4-1BB/LAG-3 co-expression in YFP⁺ or YFP⁻ CD8⁺ TILs on day 7 and 14. n=3; two independent experiments. (G) Expression of Egr3 and Hif1α in Egr2GFP^{hi} and Egr2GFP^{lo} from day 7 CD8⁺ TILs isolated from Egr2GFP mice. n=5; two-independent experiments. Error bars indicate ±SEM. *:P<0.05, **:P<0.01, ***:P<0.001. A two-way ANOVA with Bonferroni post-hoc test was used for longitudinal studies (A and C) and a Mann-Whitney test was used to compute significance in (B, D, E, F and G).

FIG. 3A-H. Co-expression of 4-1BB and LAG-3 identifies tumor antigen-specific TILs in progressing tumors. (A) Representative CDR3β distributions from the different 4-1BB/LAG-3 subpopulations and CD8⁺ T cells isolated from the spleen. Boxed regions represent dominant peaks in the 4-1BB⁺LAG-3⁺ CD8⁺ TIL subpopulation. (B) As a measure of skewness, the Hamming Distance (HD) for each Vβ spectratype was calculated between each TIL subpopulation and CD8⁺ T cell spleen population within the same mouse. As a control the HDs from CD8⁺ splenocyte populations between mice (grey bar) were calculated. n=3; one independent experiment. (C-D) Representative flow analysis of the 4-1BB/LAG-3 subpopulation in H-2K^{b}/SIY⁺ and H-2K^{b}/SIY⁻ CD8⁺ TILs on day 14 after B16.SIY and MC38.SIY or (D) MC57.SIY and 1969.SIY tumor inoculation. n=3-4; three to five independent experiments. (E) Summary of the composition of H-2K^{b}/SIY⁺ and H-2K^{b}/SIY⁻ CD8⁺ TILs co-expressing 4-1BB and LAG-3 comparing B16.SIY, MC38.SIY, MC57.SIY and 1969.SIY tumors on day 14 after tumor inoculation. n=5; three to four independent experiments. (F-H) On day 7 after tumor inoculation 1×10⁶ P14/CD45.2 and 2C/CD45.1/2 Tg T cells were adoptively transferred, via tail vein, into CD45.1 congenic tumor bearing hosts and analyzed for the (F) total number of recovered cells in the tumor, (G and H) profile of 4-1BB and LAG-3 expression in 2C, P14 and host CD8⁺ TILs. n=5; two-independent experiments. All error bars indicate ±SEM. *:P<0.05, **:P<0.01, ***:P<0.001. A Kruskal-Wallis (non-parameteric) test was used for (B) spectratype analysis and (E and F) H-2K^{b}/SIY analysis. A two-way ANOVA with Bonferroni post-hoc test was used for (H) 2C, Host and P14 composition analysis.

FIG. 4A-G. Co-expression of 4-1BB and LAG-3 but not PD-1 define dysfunctional CD8⁺ TILs with diminished IL-2. (A and B) Sorted cells from day 14 B16.SIY tumor bearing mice were stimulated in vitro with anti-CD3P and anti-CD28 for 12 hours and analyzed for (A) Il-2 transcript by qRT-PCR and (B) IL-2 protein by ELISA. Two tumors on opposite flanks pooled per mouse. n=4-5; three independent experiments. (C) Egr2GFP^{hi} and Egr2GFP^{lo} TILs were sorted from day 14 B16.SIY tumor bearing Egr2GFP mice and stimulated in vitro for 12 hours and analyzed for Il-2 transcript by qRT-PCR. Two tumors on opposite flanks pooled per mouse. n=5; two independent experiments. (D) On day 7 after tumor inoculation 1×10⁶ 2C/CD45.1/2 Tg T cells were transferred into mice, 7 days later host 4-1BB⁺LAG-3⁺ T cells sorted from the tumor and 2C T cells sorted from the tumor or TdLN were stimulated in vitro and analyzed for expression of Il-2 transcript by qRT-PCR. Two tumors on opposite flanks pooled per mouse. n=3; two independent experiments. (E and F) Representative flow analysis of PD-1 expression on 4-1BB/LAG-3 CD8⁺ TIL subpopulations and (F) summary of the composition of the 4-1BB⁻LAG-3⁻PD-1⁺ subpopulation in the CD8⁺ TIL compartment on day 14 and 21. n=5; three independent experiments. (G) 4-1BB⁻LAG-3⁻ PD-1⁺ and LAG-3⁺4-1BB+CD8⁺ TILs were sorted from day 14 tumor bearing mice, stimulated in vitro and analyzed for Il-2 transcript by qRT-PCR. Two tumors on opposite flanks pooled per mouse. n=3; two independent experiments. All error bars indicate ±SEM. *:P<0.05, **:P<0.01, ***:P<0.001 ****:P<0.0001. A Kruskal-Wallis (non-parametric) test was used for analysis of multiple comparisons (A, B, and D) and a Mann-Whitney test was used for pair-wise comparisons (C and G).

FIG. 5A-E. Dysfunctional CD8⁺ TILs retain IFN-7 production, cytolytic capacity and produce Treg-recruiting chemokines. (A) Longitudinal analysis CD8⁺ TIL subpopulation cytokine production capacity. CD8⁺ TIL subpopulations were sorted and stimulated with anti-CD3ε and anti-CD28 for 10-12 hours and the concentration of IL-2, IFN-γ and TNF-α was measured. Concentration was normalized to cell number. Two tumors on opposite flanks pooled for day 7 and 14. n=4-5; two-independent experiments. (B) Ifn-γ Tnf-α and Gzmb transcript levels in the 4-1BB/LAG-3 subpopulations analyzed directly ex vivo. Two tumors on opposite flanks pooled per mouse. n=3-5; three-independent experiments. (C) Representative flow plot and summary of IFN-7 production analyzed directly ex vivo. Briefly, 100 μl of PBS containing 2 mg/mL GolgiPlug was injected intratumorally on day 14 after tumor inoculation. 8 hours later TILs were isolated. All steps were performed on ice with media containing 1 mg/mL GolgiStop until fixation. n=5; two independent experiments. (D) CD8⁺ TIL subpopulations at indicated time points were sorted and plated with 50,000 P815 target cells and 1 μg/mL anti-CD3ε. Lysed target cells were measured by positive staining for propidium iodide and/or live/dead fixable viability dye. P815 target cells plated without CTLs were used as a negative control (black bar). Primed OTI cells were used as a positive control. Tumors from 10 mice with 2 tumors on opposite flank were pooled to obtain sufficient quantities of CD8⁺ TILs. Data are representative of three independent experiments. (E) Ccl1 and Ccl22 transcript levels in the 4-1BB/LAG-3 subpopulations analyzed directly ex vivo by qRT-PCR. n=4; two independent experiments. *:P<0.05, **:P<0.01, ***:P<0.001, ****:P<0.0001. A Kruskal-Wallis (non-parametric) test was used for (A-C, E) cytokine/chemokine analysis and a two-way ANOVA with Bonferroni post-hoc test was used for (D) cytolytic assay.

FIG. 6A-D. Dysfunctional CD8⁺ TILs express a wide range of co-inhibitory and co-stimulatory receptors. (B) Gene expression profile of cell surface receptors in the 4-1BB/LAG-3 CD8⁺ TIL subsets. Probe sets that revealed a 1.5-fold increase in the 4-1BB⁺LAG-3⁺ population relative to the 4-1BB⁻LAG-3⁻PD-1⁻ population are displayed. Columns show the log₂-transformed signal intensity. (C) Longitudinal study of selected up-regulated cell surface receptors. Flow plots are representative of the CD8⁺ TIL subsets on day 14. n=5; two to five independent experiments for each time point. (D) Representative flow plot and summary of KLRG-1 and IL-7Rα expression among the 4-1BB/LAG-3 subpopulations on day 14 after tumor inoculation. n=5; two independent experiments. *:P<0.05, **:P<0.01, ***:P<0.001, ****:P<0.0001. A two-way ANOVA with Bonferroni post-hoc test was used for all analyses.

FIG. 7A-G. Anti-4-1BB and anti-LAG-3 acts synergistically to control tumor outgrowth and restore TIL function. (A) Tumor outgrowth measured in mm². Arrows indicate on which days mice received antibody therapy. Statistical significance at indicate time points is in comparison to anti-4-1BB+anti-LAG-3 treatment. n=5; two independent experiments. (B) Composition of H-2K^{b}/SIY⁺CD8⁺ TILs on day 14. Mice received antibody doses (100 μg each) on days 7, 10, 13 and 16. n=5; two independent experiments. (C-F) Representative flow plot and summary of NRP1/2B4 (C and E) and KLRG-1/IL-7Rα (D and F) expression in H-2K^{b}/SIY⁺ CD8⁺ TILs without FTY720 (C and D) and with FTY720 (E and F) on day 14 after tumor inoculation. Mice received antibody treatment as in (A and B) and FTY720 was administered at a dose of 25 μg/mouse by gavage starting one day before treatment and continuing one dose per day until analysis (day 6 to day 13). n=5; two-independent experiments. (G) IL-2 production after treatment. Sorted cells from treated or untreated day 14 B16.SIY tumor bearing mice were stimulated in vitro for 12 hours and analyzed for Il-2 transcript by qRT-PCR. Protein concentration was determined by the bead-based LEGENDplex immunoassay and normalized to cell number. Two tumors on opposite flanks pooled per mouse. n=2-3; two independent experiments. A two-way ANOVA with Bonferroni post-hoc test was used for all analyses. *:P<0.05, **:P<0.01, ***:P<0.001.

FIG. 8. Spectratype graphs used in the analysis in FIG. 3B.

FIG. 9. CD3⁺ T cells on day 14 after FTY720 administration.

FIG. 10A-B. Statistical analysis of the cross-study comparison of gene expression profiles. (A) Rank-Rank Hypergeometric plots of each pair-wise comparison. (B) Pair-wise correlation of expression values between each data set. Rho (p) is the spearman rank correlation coefficient.

FIG. 11A-E. Nrn1, CRTAM and Sema7a are regulators of anti-tumor immunity. (A) Tumor growth measured in mm2. Nrn1^{-/-} or Sema7a^{-/-} and littermate control mice were engrafted with 2×106 B16.SIY cells subcutaneously. (B) Gene expression analysis of Nrn1 in T cell subsets of the spleen, TdLN and Tumor. (C) Representative flow plot and summary of IFN-g production of WT, Nrn^{-/-} or (D) CRTAM^{-/-} 2C T cells on day 7. Briefly, on the same day as tumor inoculation, 1×10⁶ Cell Trace Violet-labeled 2C T cells were transferred into mice by tail vein injection. On day 7, whole TdLN suspensions were restimulated with SIY peptide for 12 hours and analyzed for cell trace dilution and IFN-g production. (E) Mice that received 1×10⁶ Nrn1^{-/-} 2C T cells are more likely to exhibit complete tumor control compared to mice that received the same number of WT 2C T cells. Adoptive transfer of T cells was performed the same way as in (C).

FIG. 12. Exemplary experimental protocol and data.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a” “an” and “the” include plural reference unless the context clearly dictates otherwise. Thus, for example, reference to “an antibody” is a reference to one or more antibodies and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition (e.g., cancer, solid tumor cancer, etc.).

As used herein, an “immune response” refers to the action of a cell of the immune system (e.g., T lymphocytes, B lymphocytes, natural killer (NK) cells, macrophages, eosinophils, mast cells, dendritic cells, neutrophils, etc.) and soluble macromolecules produced by any of these cells or the liver (including antibodies, cytokines, and complement) that results in selective targeting, binding to, damage to, destruction of, and/or elimination from a subject of invading pathogens, cells or tissues infected with pathogens, or cancerous or other abnormal cells.

As used herein, the term “immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the cancer microenvironment.

As used herein, the term “immunotherapy” refers to the treatment or prevention of a disease or condition by a method comprising inducing, enhancing, suppressing or otherwise modifying an immune response.

As used herein, “potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “antibody” refers to a whole antibody molecule or a fragment thereof (e.g., fragments such as Fab, Fab′, and F(ab′)₂), unless otherwise specified (e.g., “whole antibody,” “antibody fragment”). An antibody may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, VH, and three constant regions, C_{H1}, C_{H2}, and C_{H3}. The VH domain is at the amino-terminus of the heavy chain, and the CH₃ domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “anti-4-1BB antibody” or “4-1BB antibody” refers to an antibody which specifically recognizes an antigen and/or epitope presented by 4-1BB. Similarly, the terms “anti-LAG-3 antibody” and “LAG-3 antibody” refer to an antibody which specifically recognizes an antigen and/or epitope presented by LAG-3, the terms “anti-Nrn1 antibody” and “Nrn1 antibody” refer to an antibody which specifically recognizes an antigen and/or epitope presented by Nrn1, the terms “anti-CRTAM antibody” and “CRTAM antibody” refer to an antibody which specifically recognizes an antigen and/or epitope presented by CRTAM, and the terms “anti-Sema7a antibody” and “Sema7a antibody” refer to an antibody which specifically recognizes an antigen and/or epitope presented by Sema7a. Antibodies that recognize epitopes on other molecular entities may be referred to according to a similar scheme (e.g., anti-CTLA-4, anti-PD-L1, etc.).

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

As used herein, the term “chimeric antibody” refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

A “humanized” antibody refers to a non-human antibody that has been modified so that it more closely matches (in amino acid sequence) a human antibody. A humanized antibody is thus a type of chimeric antibody. In certain embodiments, amino acid residues outside of the antigen binding residues of the variable region of the non-human antibody are modified. In certain embodiments, a humanized antibody is constructed by replacing all or a portion of a complementarity determining region (CDR) of a human antibody with all or a portion of a CDR from another antibody, such as a non-human antibody, having the desired antigen binding specificity. In certain embodiments, a humanized antibody comprises variable regions in which all or substantially all of the CDRs correspond to CDRs of a non-human antibody and all or substantially all of the framework regions (FRs) correspond to FRs of a human antibody. In certain such embodiments, a humanized antibody further comprises a constant region (Fc) of a human antibody.

The term “human antibody” refers to a monoclonal antibody that contains human antibody sequences and does not contain antibody sequences from a non-human animal. In certain embodiments, a human antibody may contain synthetic sequences not found in native antibodies. The term is not limited by the manner in which the antibodies are made. For example, in various embodiments, a human antibody may be made in a transgenic mouse, by phage display, by human B-lymphocytes, or by recombinant methods.

As used herein, the term “natural antibody” refers to an antibody in which the heavy and light chains of the antibody have been made and paired by the immune system of a multicellular organism. For example, the antibodies produced by the antibody-producing cells isolated from a first animal immunized with an antigen are natural antibodies. Natural antibodies contain naturally-paired heavy and light chains. The term “natural human antibody” refers to an antibody in which the heavy and light chains of the antibody have been made and paired by the immune system of a human subject.

Native human light chains are typically classified as kappa and lambda light chains. Native human heavy chains are typically classified as mu, delta, gamma, alpha, or epsilon, and define the antibody's isotype as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids. See, e.g., Fundamental Immunology (1989) Ch. 7 (Paul, W., ed., 2nd ed. Raven Press, N.Y.); herein incorporated by reference in its entirety.

The term “neutralizing antibody” or “antibody that neutralizes” refers to an antibody that reduces at least one activity of a polypeptide comprising the epitope to which the antibody specifically binds. In certain embodiments, a neutralizing antibody reduces an activity in vitro and/or In vivo. In some embodiments, by neutralizing the polypeptide comprising the epitope, the neutralizing antibody inhibits the capacity of the cell displaying the epitope.

As used herein, the term “glycoengineered”, as used herein, includes any manipulation of the glycosylation pattern of a naturally occurring or recombinant protein, polypeptide or a fragment thereof.

The term “antigen-binding site” refers to a portion of an antibody capable of specifically binding an antigen. In certain embodiments, an antigen-binding site is provided by one or more antibody variable regions.

The term “epitope” refers to any polypeptide determinant capable of specifically binding to an immunoglobulin or a T-cell or B-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class, whereas conservative substitutions may involve the exchange of a member of one of these classes for another member of that same class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

The term “effective dose” or “effective amount” refers to an amount of an agent, e.g., an antibody, that results in the reduction of symptoms in a patient or results in a desired biological outcome. In certain embodiments, an effective dose or effective amount is sufficient to treat or reduce symptoms of a disease or condition.

As used herein, the terms “administration” and “administering” refer to the act of giving a drug, prodrug, or other agent, or therapeutic to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

The term “treatment” encompasses both therapeutic and prophylactic/preventative measures unless otherwise indicated. Those in need of treatment include, but are not limited to, individuals already having a particular condition as well as individuals who are at risk of acquiring a particular condition or disorder (e.g., those having a genetic or epigenetic predisposition; based on age, gender, lifestyle, etc.). The term “treating” refers to administering an agent to a subject for therapeutic and/or prophylactic/preventative purposes.

A “therapeutic agent” refers to an agent that may be administered In vivo to bring about a therapeutic and/or prophylactic/preventative effect.

A “therapeutic antibody” refers to an antibody that may be administered In vivo to bring about a therapeutic and/or prophylactic/preventative effect.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term pharmaceutical composition” refers to the combination of an active agent (e.g., binding agent) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, a “diagnostic” or “diagnostic test” includes the detection, identification, or characterization of a disease state or condition of a subject. For example, a disease or condition may be characterized to determine the likelihood that a subject with a disease or condition will respond to a particular therapy, determine the prognosis of a subject with a disease or condition (or its likely progression or regression), determine the effect of a treatment on a subject with a disease or condition, or determine a future treatment course of action.

### DETAILED DESCRIPTION

Provided herein are compositions and methods for detecting and/or targeting dysfunctional tumor antigen-specific CD8⁺ T cells in the tumor microenvironment for diagnostic, therapeutic and/or research applications. In particular, dysfunctional tumor antigen-specific CD8⁺ T cells are detected and/or targeted via their expression of cell surface receptors described herein, such as 4-1BB,LAG-3, or additional markers that correlate with 4-1BB and LAG-3 expression, such as markers differentially expressed on the surface of the T cells (e.g., PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM and Sema7a).

Experiments conducted during development of embodiments herein identified markers/receptors that correlate and/or are responsible for tumor antigen-specific CD8⁺ T cell dysfunction. In some embodiments, the markers/receptors are overexpressed in dysfunctional tumor antigen-specific CD8⁺ T cells. In such embodiments, detecting the level (e.g., above a threshold) of such markers provides a diagnostic for detecting tumor antigen-specific CD8⁺ T cell dysfunction. Further, in such embodiments, targeting (e.g., inhibiting (e.g., expression and/or activity of)) such markers/receptors provides a therapeutic. In other embodiments, the markers/receptors are under expressed in dysfunctional tumor antigen-specific CD8⁺ T cells. In such embodiments, detecting the level (e.g., below a threshold) of such markers provides a diagnostic for detecting tumor antigen-specific CD8⁺ T cell dysfunction. Further, in such embodiments, targeting (e.g., enhancing (e.g., expression and/or activity of)) such markers/receptors provides a therapeutic.

Transcription factor Egr2 is a critical regulator of the anergic state in CD4⁺ T cell clones manipulated in vitro (Zheng et al., 2013; 2012; incorporated by reference in their entireties). Egr2 has also been shown to be involved in negative regulation of T cell activation in several in vivo model systems (Sumitomo et al., 2013; incorporated by reference in its entirety). Egr2 contributes to upregulation of DGKa and -z which act to blunt TCR-mediated Ras pathway activation (Zha et al., 2006; incorporated by reference in its entirety). By comparing gene expression profiling of energized cells along with Egr2 ChIP-Seq analysis multiple additional Egr2-driven gene targets were identified (Zheng et al., 2013; incorporated by reference in its entirety). These gene targets include 4-1BB (Tnfrsf9 or CD137), Lag3, Nrn1, Sema7a, Crtam, and Rank1, which encode cell surface proteins. 4-1BB is a co-stimulatory molecule transiently expressed after TCR engagement. Lag3 (lymphocyte-activation gene 3 or CD223) is a CD4 homologue and functions as an inhibitory receptor. Expression of 4-1BB and Lag3 is regulated following TCR engagement and continues throughout differentiation. In humans, 4-1BB and LAG-3 are expressed on CD8⁺ TILs from human melanoma tumors (Gros et al., 2014; Baitsch et al., 2012; incorporated by reference in their entireties). In both mice and humans, either molecule alone are expressed on populations of activated T cells. However, co-expression is more limited and is rarely observed in circulating T cells. The function of CD8⁺ TILs co-expressing these markers is unknown.

Experiments were conducted during development of embodiments herein to investigate the detailed characteristics of CD8⁺ TILs expressing 4-1BB and LAG-3 using mouse tumor models. It was found that the co-expression of 4-1BB and LAG-3 was sufficient to identify tumor antigen-specific dysfunctional CD8⁺ TILs enriched in the expression of Egr2 target genes. These CD8⁺ TILs failed to make IL-2 following in vitro stimulation, yet still produced IFN-g and Treg-recruiting chemokines and lysed target cells ex vivo, indicating they are not completely functionally inert. Combinatorial treatment with anti-LAG-3/anti-4-1BB restored the function of this population and promoted in situ acquisition of KLRG-1hi effector cells. Additional gene expression profiling provided a complete phenotyping of this T cell subset, which revealed expression of a broad panel of both inhibitory receptors and co-stimulatory receptors (e.g., receptors of Table 2 (e.g. Nrn1, Sema7a, CRTAM, etc.)). Inhibitory receptors and co-stimulatory receptors identified in this profiling that are displayed on the surface of T cells include PD-1, TIM-3, OX-40ICOS, TIGIT, CD244, TNFRSF18, Nrn1, Nrp1, KLRG1, GM156, GPNMB, GPR65, TMEM205, and TMEM126A. These approaches have thus enabled the characterization of the population of tumor antigen-specific CD8+ T cells that arise specifically within the tumor microenvironment having altered functional properties. In some embodiments, this population is a target for immunotherapeutic approaches to restore desired functionality and promote tumor regression. In some embodiments, the receptors/markers identified herein (e.g., 4-1BB, LAG-3, receptors/markers of Table 2 (e.g., surface markers/receptors (e.g. Nrn1, Sema7a, CRTAM, etc.), etc.) etc.) are targeted (e.g., via immunotherapeutic approaches) to restore desired immunoresponsiveness, to promote tumor regression, and/or for the treatment of cancer.

Experiments conducted during development of embodiments herein applied knowledge of Egr2 targets to evaluate applicability of these markers toward understanding dysfunctional T cells within tumors in vivo. The data indeed confirm that co-expression of LAG-3 and 4-1BB is sufficient to identify the majority of tumor antigen-specific CD8⁺ T cells within the tumor microenvironment. Co-expression of these markers was not observed within peripheral lymphoid organs in tumor-bearing mice, indicating that a property unique to the tumor context drives 4-1BB and LAG-3 expression. In addition, acquisition of LAG-3 and 4-1BB expression was not observed within tumors that were undergoing successful rejection, indicating that the acquisition of this phenotype occurs under conditions of incomplete antigen clearance.

In some embodiments, cancer treatment methods described herein comprise administration (or co-administration with one or more additional therapies/therapeutics) of one or more anti-4-1BB and/or anti-LAG-3 agents (e.g., antibodies, antibody fragments, antibody mimetic molecules (e.g., DARPins, affibodies, aptamers, nanobodies, etc.), etc.). In some embodiments, an anti-4-1BB and/or anti-LAG-3 agents is administered to render cancer cells, tumor(s), and/or the tumor microenvironment accessible or susceptible to treatment with additional therapies/therapeutics (e.g., immunotherapeutics). Anti-4-1BB and/or anti-LAG-3 agents that find use in embodiments described herein are not limited by their mechanism of action. Agents may be small molecules, peptide, polypeptides, proteins, nucleic acids (e.g., antisense, RNAi, etc.), antibodies, antibody fragments, etc.

In some embodiments, cancer treatment methods described herein comprise enhancing the activity or expression of a marker/receptor identified herein that negatively correlates with tumor antigen-specific CD8⁺ T cell dysfunction.

Experiments conducted during development of embodiments herein identified receptors/markers that are differentially expressed in dysfunctional CD8+ TILs (See Table 2). Testing of targets of interest identified in that screen demonstrate that at least neuritin 1 (Nrn1), cytotoxic and regulatory t-cell molecule (CRTAM), and Semaphorin 7A (Sema7a) are regulators of anti-tumor immunity, with Nrn1 and CRTAM blockade correlating with increased tumor area, and Sema7a blockade correlating with decreased tumor area.

In some embodiments, cancer treatment methods described herein comprise administration (or co-administration with one or more additional therapies/therapeutics) of agents (e.g., antibodies, antibody fragments, antibody mimetic molecules (e.g., DARPins, affibodies, aptamers, nanobodies, etc.), etc.) that target one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.). In some embodiments, an agent is administered to render cancer cells, tumor(s), and/or the tumor microenvironment accessible or susceptible to treatment with additional therapies/therapeutics (e.g., immunotherapeutics). Agents targeting one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.) that find use in embodiments described herein are not limited by their mechanism of action. Agents may be small molecules, peptides, polypeptides, proteins, nucleic acids (e.g., antisense, RNAi, etc.), antibodies, antibody fragments, etc. In some embodiments, an antagonist of Nrn1 is administered. In some embodiments, an antagonist of CRTAM is administered. In some embodiments, an agonist of Sema7a is administered.

In some embodiments, antibodies, antibody fragments, antibody mimetic molecules (e.g., DARPins, affibodies, aptamers, nanobodies, etc.) targeting 4-1BB, LAG-3 and/or one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.), or fragments thereof, are provided. Such agents may be naked, deriving their effect by target binding (e.g., neutralizing the target), or may be conjugated to a functional moiety (e.g., drug, toxin, effector moiety, etc.).

In some embodiments, a subject is treated with (i) one or more agents (e.g., antibodies, antibody fragments, antibody mimetic molecules (e.g., DARPins, affibodies, aptamers, nanobodies, etc.), etc.) that target 4-1BB, LAG-3 and/or one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.), as well as (ii) one or more additional cancer therapies. Such therapies include chemotherapy, immunotherapy, radiation, surgery, etc. In some embodiments, agents targeting the receptors/markers described herein are co-administered with one or more additional agents for the treatment of cancer.

In some embodiments, exemplary anticancer agents suitable for use in compositions and methods described herein include, but are not limited to: 1) alkaloids, including microtubule inhibitors (e.g., vincristine, vinblastine, and vindesine, etc.), microtubule stabilizers (e.g., paclitaxel (Taxol), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies (e.g., conjugated with anticancer drugs, toxins, and/or radionuclides, etc.; neutralizing antibodies; etc.); 9) biological response modifiers (e.g., interferons (e.g., IFN-.alpha., etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); and 22) modulators of p53 protein function.

In some embodiments, agents targeting 4-1BB, LAG-3 and/or one or more receptors/markers of Table 2 (e.g. Nrn1, Sema7a, CRTAM, etc.) are administered to overcome immune invasion of the cancer cells, tumor, tumor microenvironment, etc. In some embodiments, one or more additional cancer immunotherapies are employed (e.g., concurrently or serially) to make use of the immune responsiveness of the treated cells/tumor. Suitable immunotherapies may include, but are not limited to: cell-based therapies (e.g., dendritic cell or T cell therapy, etc.), monoclonal antibody (mAb) therapy (e.g., naked mAbs, conjugated mAbs), cytokine therapy (e.g., interferons, interleukins, etc.), adjuvant treatment (e.g., polysaccharide-K), etc.

In some embodiments, agents targeting 4-1BB, LAG-3 and/or one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.) are co-administered with agents (e.g., small molecules, peptides, antibodies, antibody fragments, etc.) that target one or more cancer cell or tumor) markers or components. In some embodiments, such co-administration renders the cancer cells, tumor, and/or tumor microenvironment susceptible and/or accessible to the treatment with the additional agent.

In some embodiments, agents for use in the methods and compositions described herein target and/or binds a cancer or tumor cell marker or component, selected from the group including but not limited to, epidermal growth factor receptor (EGFR, EGFR1, ErbB-1, HER1). ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-β), TGF-β receptor; Interleukin β receptor alpha2 chain (IL13Ralpha2), Interleukin-6 (IL-6), 1L-6 receptor, interleukin-4, IL-4 receptor, Cytokine receptors, Class I (hematopoietin family) and Class II (interferon/1L-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT12, SNRPD1, SYT-SSX1 or -SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl glucosaminyl transferase V, MGAT5), HERV-K MEL, KK-LC, KM-HN-1, LAGE, LAGE-1, CTL-recognized antigen on melanoma (CAMEL), MAGE-A1 (MAGE-1). MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (SiLV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikrein 4, mammaglobin-A, OA1, prostate specific antigen (PSA), prostate specific membrane antigen, TRP-1/, 75. TRP-2 adipophilin, interferon inducible protein absent in melanoma 2 (AIM-2). BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, PRAME, PSMA, RAGE-1, RNF43, RU2AS, SOX10, STEAPI, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15ql4, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enolase, heat shock protein gp96. GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), etc.

Examples of antibodies which can be incorporated into compositions and methods disclosed herein include, but are not limited, to antibodies such as trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb): Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (@(17-1A)) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym). Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals—Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab, or ImmuRAIT-CEA.

In some embodiments, an agent that finds use in embodiments herein specifically binds a component of a regulatory T cell, myeloid suppressor cell, or dendritic cell. In another aspect, the targeting moiety specifically binds one of the following molecules: CD4; CD25 (IL-2a receptor; IL-2αR); cytotoxic T-lymphocyte antigen-4 (CTLA-4; CD152); Interleukin-10 (IL-10); Transforming growth factor-beta receptor (TGF-βR); Transforming growth factor-beta (TGF-β); Programmed Death-1 (PD-1); Programmed death-1 ligand (PD-L1 or PD-L2); Receptor activator of nuclear factor-κB (RANK); Receptor activator of nuclear factor-κB (RANK) ligand (RANKL); LAG-3; glucocorticoid-induced tumor necrosis factor receptor family-related gene (GITR; TNFRSF18); or Interleukin-4 receptor (IL-4R). In some embodiments, the agent is an agonist that increases the function of the targeted molecule. In other embodiments, the agent is an antagonist that inhibits the function of the targeted molecule.

In some embodiments, an agent that finds use in embodiments herein binds a specific cytokine, cytokine receptor, co-stimulatory molecule, co-inhibitory molecule, or immunomodulatory receptor that modulates the immune system. In another aspect, the targeting moiety specifically binds one of the following molecules: tumor necrosis factor (TNF) superfamily; tumor necrosis factor-a (TNF-α); tumor necrosis factor receptor (TNFR) superfamily; Interleukin-12 (IL-12); IL-12 receptor; 4-1BB (CD137); 4-1BB ligand (4-1BBL; CD137L); OX40 (CD134; TNR4); OX40 ligand (OX40L; CD40; CD40 ligand (CD40L); CTLA-4; Programmed death-1 (PD-1); PD-1 ligand I (PD-L1: B7-H1); or PD-1 ligand 2 (PD-L2; B7-DC); B7 family; B7-1 (CD80); B7-2 (CD86); B7-H3; B7-H4; GITR/AITR: GITRL/AITRL; BTLA; CD70; CD27; LIGHT; HVEM: Toll-like receptor (TLR) (TLR 1, 2, 3, 4, 5, 6, 7, 8, 9, 10). In some embodiments, the agent is an agonist that increases the function of the targeted molecule. In other embodiments, the agent is an antagonist that inhibits the function of the targeted molecule.

In some embodiments, agents (e.g., immunotherapeutics) targeting 4-1BB, LAG-3 and/or one or more receptors/markers of Table 2 (e.g. PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.) are co-administered (e.g., serially or sequentially) with one or more adjuvants. Suitable adjuvants include, but are not limited to, one or more of: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. In addition to variant B7-DC polypeptides, other co-stimulatory molecules, including other polypeptides of the B7 family, may be administered. Proteinaceous adjuvants may be provided as the full-length polypeptide or an active fragment thereof, or in the form of DNA, such as plasmid DNA.

Pharmaceutical and immunotherapeutic compositions described herein may be delivered by any suitable route of administration (e.g., oral delivery, parenteral delivery, mucous membrane delivery, pulmonary delivery, intravenous delivery, etc.). Appropriate formulations for such delivery routes are understood in the field.

Non-limiting examples of cancers that may be treated with the compositions and methods described herein include, but are not limited to: melanoma (e.g., metastatic malignant melanoma), renal cancer (e.g. clear cell carcinoma), prostate cancer (e.g. hormone refractory prostate adenocarcinoma), pancreatic cancer (e.g., adenocarcinoma), breast cancer, colon cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, cancer is a solid tumor cancer.

Some embodiments described herein are particularly useful for the treatment of tumors that do not otherwise respond to immunotherapeutic approaches. In some embodiments, provided herein is the treatment of cancers that are non-responsive (or have a reduced response) to T cells or antigen presenting cells (e.g., dendritic cells (e.g., CD103⁺DCs, etc.), etc.). In some embodiments, provided herein is the treatment of cancers that are non-responsive to treatments, despite T cell infiltration. In some embodiments, compositions and methods described herein find use in the treatment of cancers in which T cells are not appropriately primed against tumor-associated antigens. In some embodiments, compositions and methods described herein find use in the treatment of cancers comprising tumors or cells that are defective in recruitment of dendritic cells (e.g., CD103⁺DCs, etc.). In some embodiments, compositions and methods described herein find use in the treatment of cancers comprising tumors or cells that are defective in production of the chemokine CCL4.

In some embodiments, the therapeutic compositions and methods herein find use with those described in, for example WO 2016/141312; incorporated by reference in its entirety.

In some embodiments, methods are provided for testing samples (e.g., cell, tissue, population of cells, tumor, blood, urine, saliva, etc.) from a subject for one or more biomarkers (e.g., biomarkers of dysfunctional tumor antigen-specific CD8⁺ T cells). Such biomarkers may comprise nucleic acids, small molecules, proteins, peptides, etc., and may be detected using any suitable assay technique. In some embodiments, provided herein are DNA-, RNA-, small molecule, and/or protein-based diagnostic methods that either directly or indirectly detect the biomarkers of the evasion of immune response or immunotherapy by cancer cells or tumors. The present invention also provides compositions, reagents, and kits for such diagnostic purposes.

In some embodiments, biomarkers are detected at the nucleic acid (e.g., RNA) level. For example, the presence or amount of biomarker nucleic acid (e.g., mRNA) in a sample is determined (e.g., to determine the presence or level of biomarker expression). Biomarker nucleic acid (e.g., RNA, amplified cDNA, etc.) may be detected/quantified using a variety of nucleic acid techniques known to those of ordinary skill in the art, including but not limited to nucleic acid sequencing, nucleic acid hybridization, nucleic acid amplification (e.g., by PCR, RT-PCR, qPCR, etc.), micorarray, Southern and Northern blotting, sequencing, etc. Non-amplified or amplified nucleic acids can be detected by any conventional means. For example, in some embodiments, nucleic acids are detected by hybridization with a detectably labeled probe and measurement of the resulting hybrids. Nucleic acid detection reagents may be labeled (e.g., fluorescently) or unlabeled, and may by free in solution or immobilized (e.g., on a bead, well, surface, chip, etc.).

In some embodiments, biomarkers are detected at the protein level. For example, the presence or amount of biomarker protein in a sample is determined (e.g., to determine the presence or level of biomarker expression or localization). In some embodiments, reagents are provided for the detection and/or quantification of biomarker proteins. Suitable reagents include primary antibodies (e.g., that bind to the biomarkers), secondary antibodies (e.g., that bind primary antibodies), antibody fragments, aptamers, etc. Protein detection reagents may be labeled (e.g., fluorescently) or unlabeled, and may be free in solution or immobilized (e.g., on a bead, well, surface, chip, etc.).

In some embodiments, biomarker capture reagents are provided to localize, concentrate, aggregate, etc. a biomarker. For example, in some embodiments a biomarker capture reagent that interacts with the biomarker is linked to a solid support (e.g., a bead, surface, resin, column, and the like) that allows manipulation by the user on a macroscopic scale. Often, the solid support allows the use of a mechanical means to isolate and purify the biomarker from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target) from the heterogeneous solution. Magnetic beads used to isolate targets are described in the art, e.g., as described in European Patent Application No. 87309308, incorporated herein in its entirety for all purposes.

Compositions for use in the diagnostic methods or testing steps described herein include, but are not limited to, probes, amplification oligonucleotides, and antibodies. Any of the detection and/or diagnostic reagents used in embodiments described herein may be provided alone or in combination with other compositions in the form of a kit. Kits may include any and all components necessary or sufficient for assays including, but not limited to, the detection reagents, buffers, control reagents (e.g., tissue samples, positive and negative control sample, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered.

In some embodiments, a computer-based analysis program is used to translate the raw data generated by the detection assay (e.g., the presence, absence, or amount of expression a biomarker) into data of predictive value for a clinician. In some embodiments, computer analysis combines various data into a single score or value that is predictive and/or diagnostic.

The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject. Contemplated herein are any methods capable of receiving, processing, and transmitting the information to and from laboratories conducting the assays, information providers, medical personnel, and subjects.

For example, in some embodiments of the present invention, a sample (e.g., a biopsy, cell, or blood sample) is obtained from a subject and submitted to a profiling service (e.g., clinical lab at a medical facility, third-party testing service, genomic profiling business, etc. to generate raw data. Where the sample comprises a tissue or other biological sample, the subject may visit a medical center to have the sample obtained and sent to the profiling center, or subjects may collect the sample themselves and directly send it to a profiling center. In some embodiments, a report is generated (e.g., by a clinician, by a testing center, by a computer or other automated analysis system, etc.). A report may contain test results, diagnoses, and/or treatment recommendations.

### EXPERIMENTAL

### Materials and Methods

### Mice and Tumor Inoculation

Female C57BL/6 mice ranging from 6 to 8 weeks were purchased from Taconic Farms. CD45.1 and Rag2^{-/-} mice on the C57BL/6 background were obtained from Taconic Farms and bred at the University of Chicago. 2C/Rag2^{-/-} and P14/Rag2^{-/-} mice have been previously described (Brown et al., 2006; incorporated by reference in its entirety). pLCK-CreERT2×ROSA-YFP mice were generated and have been described (Evaristo et al., 2016; incorporated by reference in its entirety). B16.SIY.dsRed (Kline et al., 2012; incorporated by reference in its entirety), C1498.SIY.GFP (Zhang et al., 2009; incorporated by reference in its entirety), and MC57.SIY.GFP (Spiotto et al., 2002; incorporated by reference in its entirety) tumor cells were engineered to express either dsRed or GFP in frame with the H2-K^{b}-restricted model antigen SIYRYYGL. The 1969.SIY.GFP cell line was engineered by retroviral transduction of the 1969 cell line (Diamond et al., 2011; incorporated by reference in its entirety) using the pLEGFP plasmid expressing cDNA for SIYRYYGL (Spiotto et al., 2002; incorporated by reference in its entirety). For experiments, mice 6 to 9 weeks of age received 2×10⁶ tumor cells subcutaneously on either the left flank or both the left and right flank. All mice were maintained according to the National Institute of Health Animal Care guidelines and studied under IACUC-approved protocols.

To generate the targeting construct for the Egr2^{EGFP} knock-in reporter mice, a 12.6kb mouse genomic DNA fragment including the egr2 gene was excised with SacII and cloned into a pEasy-Flox vector adjacent to the thymidine kinase (TK) selection marker. A cassette containing IRES2-eGFP and a LoxP-flanked neomycin selection marker was inserted into an Nhel site between the translation stop codon (TGA) and the polyadenylation signal of the egr2 gene. ES cell clones from 129 mice were electroporated and selected for Neomycin resistance. ES cell clones were verified for homologous insertion in the endogenous locus by PCR and southern blot with 5′ and 3′ probes. Mice were backcrossed to C57BL/6 for over 8 generations.

### TIL Isolation

Tumors were harvested from mice at the indicated time points. Tumors were dissociated through a 50 m filter and washed with PBS. TTLs were further enriched by layering Ficoll-Hypaque beneath the cell suspension followed by centrifugation without breaks for 30 min at 400×g. The buffy layer was isolated and washed twice with PBS before staining. For isolating specific cell populations by FACS, tumors were pooled when indicated and the cell layer was re-purified by Ficoll-Hypaque centrifugation twice. For day 28 tumors, after Ficoll-Hypaque separation, T cells were further purified by negative bead selection according to manufacturer's instructions (MAGNISORT, eBiosciences). Cells were then washed with PBS, stained at 4° C. for 15 minutes before resuspending in complete DMEM (cDMEM: 10% FBS, 100 U/mL Penicillin-Streptomycin, 1% MEM Non-Essential Amino Acids, 50 μM β-ME, 0.01M MOPS), and were sorted into either RLT lysis buffer (QIAGEN) or cDMEM depending on the experimental assay. Cells sorted into RLT buffer were put directly on dry ice as soon as the sort was finished.

### Flow Cytometry and Antibodies

Cell suspensions were washed twice in PBS before staining an FACS buffer (10% FBS, 2 mM EDTA, 0.001% NaN₃). Cells were stained for 30 min on ice and fixed in 1% PFA. Antibodies against the following molecules were used: CD3 (17A2, AX700), 2B4 (2B4, FITC), CD127 (A7R34, PE), OX-40 (OX-86, PE), 4-1BB (17B5, Biotin, APC), CD160 (7H1, PE-Cy7), LAG-3 (C9B7W, PerCPeFluor7l0), PD-1 (RMP1-30, PE-Cy7), NRP1 (3E12, BV421), GITR (DTA-1, FITC), ICOS (7E.17G9, BV421), KLRG-1 (2F1, eF450, BV605), TIGIT (1G9, APC), TIM-3 (RMT3-23, PE), CD4 (RM4-5, BV605), CD45.1 (A20, FITC), CD45.2 (104, PE), CD8a (53-6.7, BV711). Fixable Viability Dye 506 (eBioscience) was used for live/dead discrimination. Staining of SIY-specific T cells was performed utilizing the SIYRYYGL-Pentamer (PE) (Proimmune); a SIINFEKL-pentamer (PE) was used as a non-specific control. All flow cytometric analysis was conducted on an LSRFortessa (BD) and analyzed using FlowJo software (Tree Star).

### Quantitative Real-Time PCR

Total RNA was extracted from sorted cell populations using the RNEasy Micro Kit (QIAGEN) following the manufacturer's protocol. cDNA was synthesized using the High-Capacity cDNA Reverse Transcription kit (Applied Biosystems) according to manufacturer's instructions. Transcript levels were determined using primer-probe sets (Tables 1a and 1b) developed through the online ProbeFinder Software and the Universal Probe Library (Roche) with the exception of IL-2 (Mm00434256_ml) and 18S (Hs99999901_s1). To minimize batch effect, when possible, all samples probed for a gene were run on the same 96-well qRT-PCR plate. All primer-probe sets either contained a primer spanning an exon-exon boundary or primers spanning an intron. Expression levels of transcripts were normalized to 18S expression.

TABLE 1a
Primer Sequences

| # | Wilson | IMGT | Sequence |
|---|---|---|---|
| 0 | Cβ1.1 | TRBC1 | CTCAAACAAGGAGACCTTGGGTGG |
| 1 | Vβ1 | TRVB5 | CAGACAGCTCCAAGCTACTTTTAC |
| 2 | Vβ2 | TRVB1 | ATGAGCCAGGGCAGAACCTTGTAC |
| 3 | Vβ3 | TRVB26 | GAAATTCAGTCCTCTGAGGCAGGA |
| 4 | Vβ4 | TRVB2 | CTAAAGCCTGATGACTCGGCCACA |
| 5 | Vβ5.1 | TRVB12-2 | CTTTGGAGCTAGAGGACTCTGCCG |
| 6 | Vβ5.2 | TRVB12-1 | CCTTGGAACTGGAGGACTCTGCTA |
| 7 | Vβ6 | TRVB19 | GCCCAGAAGAACGAGATGGCCGTT |
| 8 | Vβ7 | TRVB29 | GGATTCTGCTAAAACAAACCAGACATCTGT |
| 9 | Vβ8.1 | TRVB13-3 | GCTTCCCTTTCTCAGACAGCTGTA |
| 10 | Vβ8.2 | TRVB13-2 | GCTACCCCCTCTCAGACATCAGTG |
| 11 | Vβ8.3 | TRVB13-3 | GGCTTCTCCCTCTCAGACATCTT |
| 12 | Vβ9 | TRVB17 | CTCTCTCTACATTGGCTCTGCAGG |
| 13 | Vβ10 | TRVB4 | CTTCGAATCAAGTCTGTAGAGCCG |
| 14 | Vβ11 | TRVB16 | TGAAGATCCAGAGCAGCGGGCCCC |
| 15 | Vβ12 | TRVB15 | CCACTCTGAAGATTCAACCTACAGAACCC |
| 16 | Vβ13 | TRVB14 | CAAGATCCAGTCTGCAAAGCAGGG |
| 17 | Vβ14 | TRVB31 | GCACGGAGAAGCTGCTTCTCAGCC |
| 18 | Vβ15 | TRVB20 | GCATATCTTGAAGACAGAGGC |
| 19 | Vβ16 | TRVB3 | CTCTGAAAATCCAACCCACAGCACTGG |
| 20 | Vβ17 | TRVB24 | TCTGAAGAAGACGACTCAGCACTG |
| 21 | Vβ18 | TRVB30 | GCAAGGCCTGGAGACAGCAGTATC |

TABLE 1b
Primer/Probe

| | | | Roche |
|---|---|---|---|
| Gene | Primer1 | Primer2 | Probe # |
| Lag3 | tgctttgggaagctccagt | gctgcagggaagatggac | 79 |
| Tnfrsf9 | ccggtcttaagcacagacct | gaacggtactggcgtctgtc | 108 |
| Egr2 | CTACCCGGTGGAAGACCTC | AATGTTGATCATGCCATCTCC | 60 |
| Sema7a | tcaatcggctgcaagatgt | cgcagacagctgagtagttcc | 15 |
| Crtam | AGATCCAACAACGAGGAGACA | TCATGCAACGCTTAGACTGG | 71 |
| Ccl1 | tcaccatgaaacccactgc | agcagcagctattggagacc | 71 |
| Ngn | caccctagectaacctcaacc | tgaaaacctcctcccctctt | 45 |
| Arl3 | ctggcagatccagtcctgtt | acccagttcatgccatcct | 100 |
| Exph5 | atgagggaggagagcggtat | cagcttgttgtccaaatcgtc | 67 |
| Fhl2 | agaaaaccatcatgccaggt | acaggtgaagcaggtctcgt | 74 |
| Nrn1 | atcctcgcggtgcaaata | gcccttaaagactgcatcaca | 108 |
| Ptgfrn | ccggggagatctcatcaaa | tcgaaggccatgtcatctg | 12 |
| Rankl | tgaagacacactacctgactcctg | cccacaatgtgttgcagttc | 88 |
| Hif1a | gctgctcactgtgaaggaagt | tggggaatgcattttaccat | 2 |
| Egr3 | caatctgtaccccgaggaga | ccgatgtccatcacattctct | 74 |
| Tnfa | ctgtagcccacgtcgtagc | ttgagatccatgccgttg | 25 |
| Gzmb | gctgctcactgtgaaggaagt | tggggaatgcattttaccat | 2 |
| Ccl1 | tcaccatgaaacccactgc | agcagcagctattggagacc | 71 |
| Ccl22 | tcttgctgtggcaattcaga | gcagagggtgacggatgtag | 74 |

### In Vivo Proliferation Assay

In vivo proliferation was measured by a BrdU pulse 24 hours prior to flow cytometric analysis. Each mouse received 0.8 mg BrdU injected i.p. on day 12 after tumor inoculation. TILs were isolated and surface stain was performed as described above. Following surface staining, cells were fixed and permeabilized using the Foxp3 staining kit (BD), according to manufacturer's protocol, and incubated with 100 μl PBS/DNase solution (300 μg/ml) for 30 minutes at 37° C. Cells were washed and incubated for 30 minutes at room temperature with anti-BrdU (FITC, Bu20a) and then washed with and resuspended in PBS.

### In Vitro Stimulation Assays

Tissue culture-treated 96-well round bottom plates were coated with anti-CD3F (1 μg/ml; 2C11) in DPBS overnight at 4° C. or for 2 hours at 37° C. Cells were sorted into cold cDMEM media and put on ice as soon as the sort was finished. Cells were then pelleted, resuspended in 50 μl cDMEM and incubated with soluble anti-CD28 (2 μg/ml; PV-1) for 10-12 hours for a final volume of 100 μl. After stimulation supernatants were removed for ELISA or bead-based immunoassay (LegendPlex), and cells were washed once with DPBS and resuspended in 15 μl of RNAlater Stabilization Solution (QIAGEN) or 300 μl of RLT buffer. Cells were stored at −80° C. until RNA isolation was performed.

### Protein Quantification

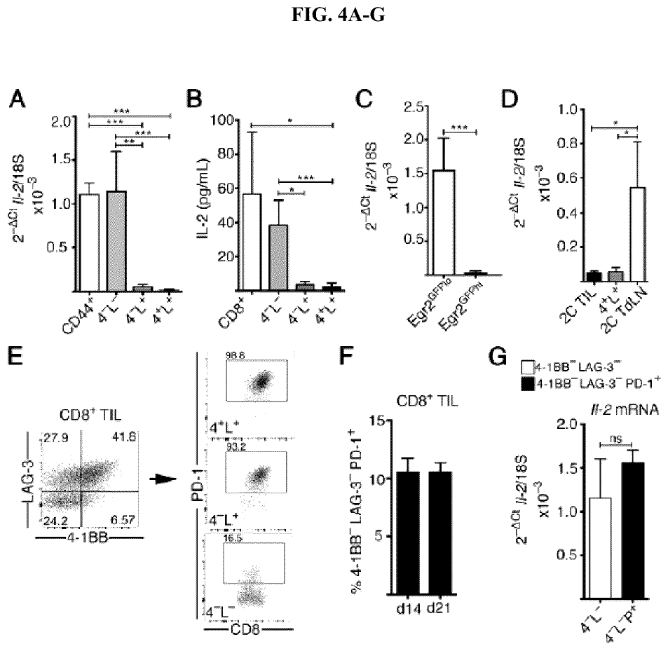

Measurement of protein concentration was determined either by a standard ELISA or bead-based immunoassay (LEGENDplex, BioLegend). ELISAs were performed according to manufacturer's protocol (Ready-SET-Go ELISA; eBioscience) on supernatants from in vitro stimulations. Absorbance values were obtained at 450 nm using an Emax microplate reader (Molecular Devices) and IL-2 concentration was determined by standard curve. Protein concentration values were normalized to the number of sorted cells plated. LEGENDplex assays were performed according to manufacturer's protocols. IL-2 concentration (FIG. 4B) was confirmed by both methods in separate experiments with no significant difference in IL-2 concentration between the two methods.

### Spectratype Analysis and Sequencing

Mice were injected with 2×10⁶ B16.SIY.dsRed tumor cells. 14 days later, tumors were harvested and specific CD8⁺ TIL subpopulations were sorted into RLT buffer (QIAGEN) and immediately frozen. cDNA was synthesized from sorted cell populations and CDR3 regions were amplified by PCR with 21 different Vβ-5′ primers paired with a FAM-Cβ1.1 primer (Table 1). Three Vβ PCR reactions did not reach significant amplification for analysis and were removed from the analysis. For sequencing, Cβ-Vβ PCR products were purified using the QIAquick PCR purification kit (QIAGEN) and sequenced at the University of Chicago Genomics Core Facility. Cβ-Vβ PCR products were analyzed by capillary electrophoresis at the University of Chicago Genomics core and CDR3 peaks were aligned using the Liz500 ladder. Spectratype graphs were displayed using the GeneiousR9 software (Kearse et al., 2012). To generate the frequency profile for each V3 spectratype, the area under each peak was measured using peak studio (fodorlab.uncc.edu/software/peakstudio). The Hamming Distance (Currier and Robinson, 2001; incorporated by reference in its entirety) was calculated between each Vβ spectratype from each CD8⁺ spleen and TIL population within a given mouse. To determine significance between the HD from each comparison the HDs for each Vβ from mice were averaged and a One-Way ANOVA with Dunn's correction for multiple comparisons was performed.

### TCR Transgenic T Cell Transfer Experiments

Cell suspensions were generated from spleens and lymph nodes from congenic 2C/Rag2^{-/-}/CD45.1/2 and/or P14/Rag2^{-/-}/CD45.2 mice and T cells were purified by CD8⁺ negative selection (Miltenyi Biotechnologies) over magnetic columns according to the manufacturer's protocol. TCR Transgenic (Tg) T cells were washed with PBS, resuspended at a concentration of 10×10⁶/ml and 1×10⁶ TCR Tg cells were adoptively transferred into CD45.1 tumor bearing mice by tail vein transfer in a volume of 0.1 mL. After indicated times, 2C T cells and corresponding host CD8⁺ T cells were sorted and stimulated as described above.

### In Vitro Cytotoxicity Assay

Per individual experiment, 10 C57BL/6 mice were injected s.c. with 2×10⁶ B16.SIY cells on both left and right flanks. On day 14, all 20 tumors were pooled and dissociated using the Tumor Dissociation Kit (Miltenyi Biotec) following the manufacturer's protocol. Tumor cell suspensions were washed 3-5 times with PBS and TILs were enriched by Ficoll-Hypaque gradient centrifugation. TILs were stained, sorted and put directly on ice. TILs were titrated and added directly to a 96-well plate containing 50,000 P815 mastocytoma cells and 1 μg/mL anti-CD3. For a positive control, OT-I cells were isolated from OT-I/Rag2^{-/-} mice and stimulated with plate-bound anti-CD3 (0.25 μg/mL), anti-CD28 (2 μg/mL) and 100 U/mL IL-2 for 2-3 days. For a negative control, P815 cells were cultured alone or cultured with naïve CD8⁺ T cells isolated from lymph nodes. After 12 hours of incubation, cells were stained for Thy1, CD45, CD8a, Fixable Viability Dye 450 (eBioscience) and/or propidium Iodide.

### Gene Expression Analysis

Total RNA for the CD8⁺ TIL subpopulations was isolated following the manufacturer's protocol (RNEasy Micro Kit: QIAGEN) from sorted cells pooled from 10 mice. Samples were analyzed by the University of Chicago Genomics Facility using Illumina MouseRef8 microarray chips. Two experimental replicates were performed, and the results were log₂ transformed and averaged. Probe sets that revealed a 1.5-fold difference abs(log 2(ratio)>1.5)) relative to CD8⁺4-1BB⁻LAG-3⁻PD-1⁻ cells were identified and used for subsequent analysis. The microarray data are available in the Gene Expression Omnibus database (ncbi.nlm.nih.gov/gds) under accession number GSE79919. For cross-study comparisons, log 2-fold change values were extracted using the GEO2R online software from the hypofunctional CD8⁺ TIL data set, GSE79858 ((GSM2107353, GSM2107353 and GSM2107355) versus (GSM2107350, GSM2107351, GSM210732)) and the CD8⁺ T cell exhausted data set, GSE41870 ((GSM1026819, GSM1026820, GSM1026821) versus (GSM1026786, GSM1026787, GSM1026788, GSM1026789)). Upregulated genes showing a 2-fold difference were used for analysis. Multiple genes names with from the GEO2R extracted data were identified and matched to gene names from the Illumina data set. The rank-rank hypergeometric overlap (RRHO) analysis (Plaisier et al., 2010; incorporated by reference in its entirety) was conducted at systems.crump.ucla.edu/rankrank/index.php and the associated Bioconductor package “RRHO” (Rosenblatt and Stein, 2014; incorporated by reference in its entirety).

### Gene Ontology Enrichment Analysis

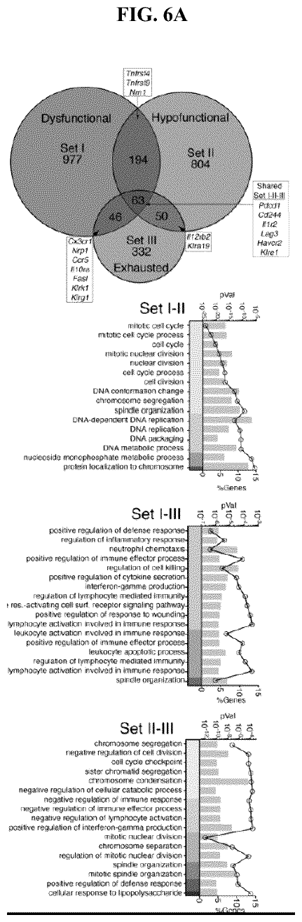

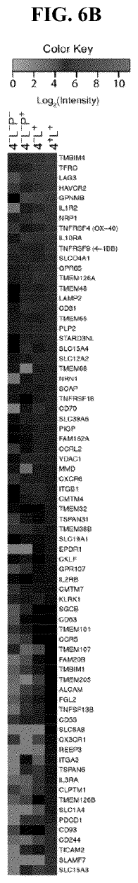

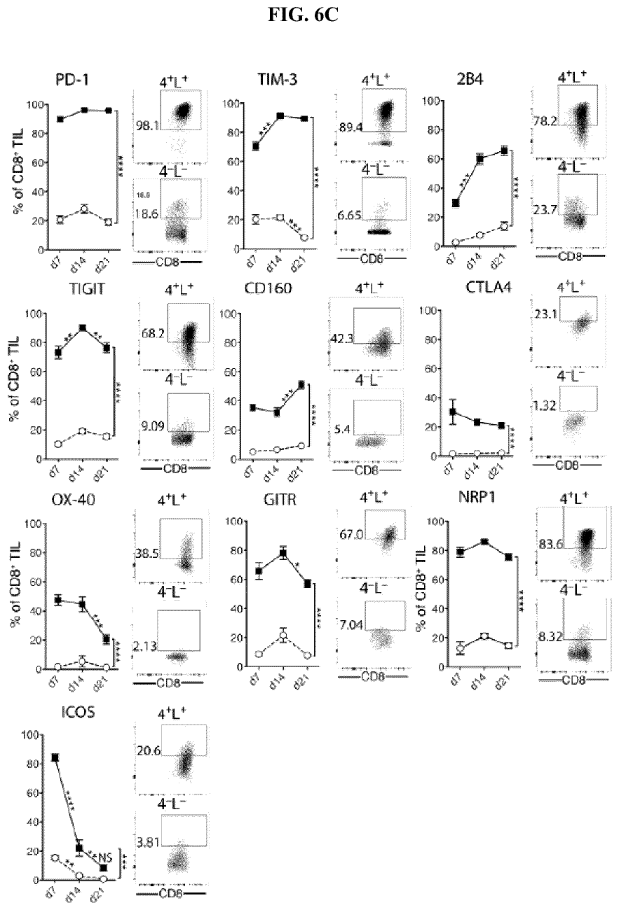

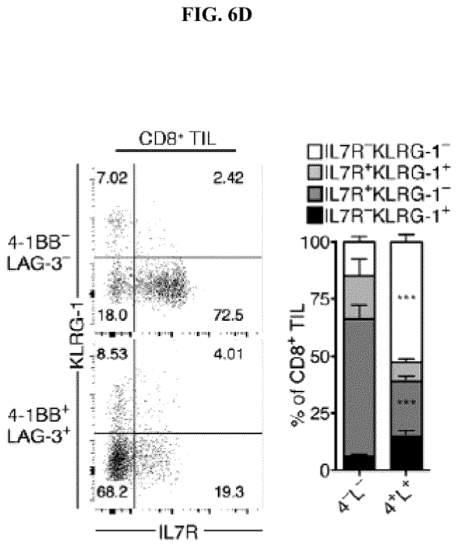

In a pair-wise fashion, shared upregulated genes were used as the input for the ClueGO software with the Cytoscape application (Shannon et al., 2003; incorporated by reference in its entirety). Both the Biological Process and Immune System Process Gene Ontology Annotations were used for analysis. Only pathways with a Bonferroni step down correction p-value>0.01 were considered when generating pathway nodes. Non-redundant pathways with the greatest number of genes found within each node were used as examples in FIG. 6A.

### Antibody and FTY720 Treatments

Mice were treated i.p. with 100 μg/mouse of anti-4-1BB (Bio-X-Cell; LOB12.3) antibody and/or 100 μg/mouse anti-LAG-3 (Bio-X-Cell; C9B7W). For tumor outgrowth experiments, mice were treated on day 7, 10, 13 and 16 after tumor inoculation. For ex vivo functional experiments mice were treated on day 7, 10 and 13 and cells were sorted on day 14. For experiments blocking lymph node egress, 25 μg of FTY720 was given by gavage one day prior to first antibody treatment (day 6) and continued every day until endpoint on day 14.

### Results

### 4-1BB and LAG-3 Identify a Major Population of CD8⁺ TILs

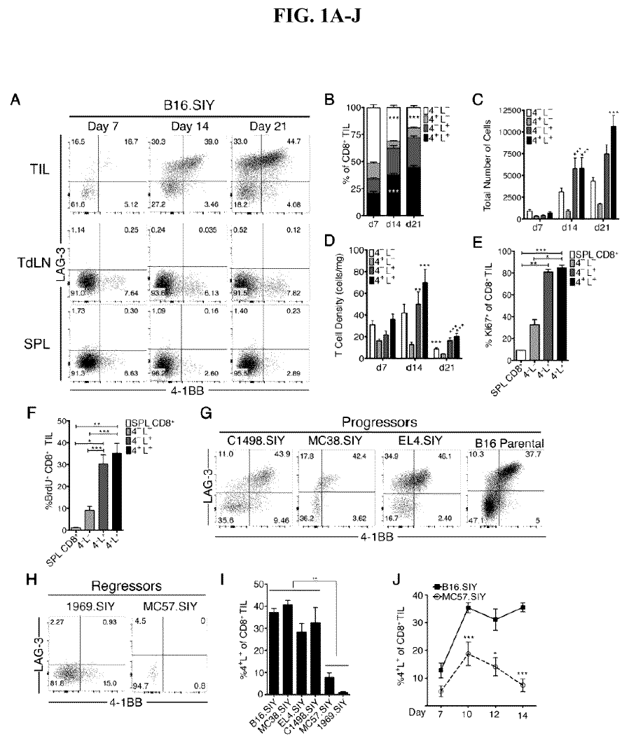

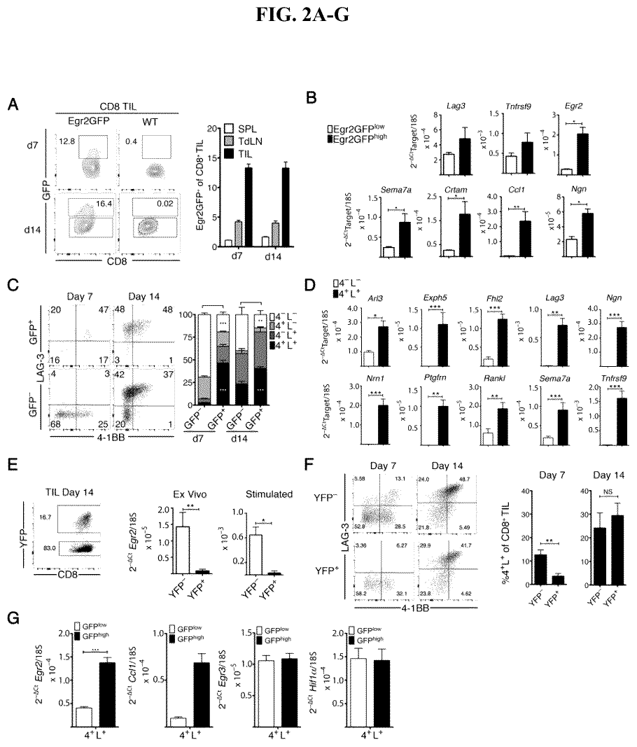

To determine whether 4-1BB and LAG-3 could identify dysfunctional CD8⁺ TILs, the expression pattern of LAG-3 and 4-1BB was examined using the well-characterized B16.SIY model of melanoma. On day 7 following tumor inoculation, the 4-1BB+LAG-3⁺ population comprised 15.8% of all CD8⁺ TILs. The frequency of this population significantly increased to 44% by day 21. The frequency of 4-1BB⁻LAG-3⁺ (4-L+) population also increased 1.9-fold from day 7 to day 14 to comprise 25% of the CD8⁺ TIL compartment. In contrast, the frequency of the 4-1BB⁻LAG-3⁻ (4⁻L⁻) population decreased by 2.7-fold by day 21. There was no significant increase in the proportion or number of 4-1BB⁺LAG-3⁻ CD8⁺ TILs within the time frame of the experiment (FIGS. 1A and B). Similar patterns were seen when analyzing absolute numbers of cell subsets (FIGS. 1C and D). Acquisition of these phenotypes was specific for the tumor microenvironment, as they were not observed in the spleen or tumor-draining lymph node (TdLN) (FIG. 1A). These data indicate that the tumor microenvironment preferentially supports the induced co-expression of LAG-3 and 4-1BB.

The selective increase in cell numbers and proportional shift towards the 4-1BB⁻LAG-3⁺ and 4-1BB⁺LAG-3⁺ populations during tumor progression indicated that expansion of these populations was occurring within the tumor microenvironment. CD8⁺ TILs were stained for Ki67 at day 14 after tumor inoculation and analyzed by flow cytometry. 81% of 4-1BB⁻LAG-3⁺ cells and 85% of 4-1BB⁺LAG-3⁺ cells were Ki67⁺ compared to only 32% of the 4-1BB⁻LAG-3⁻ TILs (FIG. 1E). Mice were pulsed with BrdU on day 12, and 24 hours later the CD8⁺ TIL subpopulations were analyzed for BrdU incorporation. Indeed, the 4-1BB⁻LAG-3⁺ and 4-1BB⁺LAG-3⁺ populations incorporated more BrdU compared to the 4-1BB⁻LAG-3⁻ population (FIG. 1F). These data indicate that once CD8⁺ T cells arrive at the tumor site, a fraction of TILs expands within the tumor, and that these expanding TILs are identified by increased expression of 4-1BB and LAG-3.

To determine if upregulation of LAG-3 and 4-1BB was simply a product of the B16.SIY tumor model or if it is a more general feature of CD8⁺ T cells within tumors, T cells from three additional progressively growing tumor models, C1498.SIY, MC38.SIY, EL4.SIY and B16F10 parental were analyzed. TILs were analyzed for expression of 4-1BB and LAG-3 at day 14. We found that the pattern of expression was similar to that seen in CD8⁺ TILs isolated from B16.SIY tumors (FIGS. 1G and I). The results from the B16F10 parental tumor confirm that presence of SIY is not required to see co-expression of 4-1BB and LAG-3. In order to determine whether the 4-1BB⁺LAG-3⁺ TIL subset was generated only in progressing tumors or also in tumors that were rejected, T cell phenotypes in the 1969.SIY and MC57.SIY fibrosarcoma tumor models we analyzed, which are more immunogenic and undergo spontaneous rejection. Distinctly fewer 4-1BB⁺LAG-3⁺ cells were found among the CD8⁺ TIL compartment in the 1969.SIY and MC57.SIY tumors (FIG. H and I). Over time, co-expression of 4-1BB and LAG-3 was maintained in B16.SIY tumors but not MC57.SIY tumors (FIG. 1J). These data indicate that the acquisition of the LAG-3⁺4-1BB⁺ TIL phenotype preferentially occurs within the tumor microenvironment and only upon conditions of tumor progression rather than regression.

CD8⁺4-1BB⁺LAG-3⁺ TILs Express Egr2 and Multiple Egr2 Gene Targets

Experiments conducted during development of embodiments herein to determine whether Egr2 expression itself was also characteristic of T cells within the CD8⁺ TIL compartment; an Egr2-IRES-GFP (Egr2^{GFP}) knock-in reporter mouse was utilized. Approximately 14% of all CD8⁺ TILs were GFP⁺ on both day 7 and day 14 (FIG. 2A). To confirm that Egr2 is faithfully reported, CD8⁺ TILs expressing high and low levels of EGFP were sorted and screened for Egr2 and several Egr2 targets by qRT-PCR. The Egr2-GFP^{hi} population expressed greater levels of Egr2 and many Egr2-target genes previously defined using in vitro anergy models. These include Tnfrsf9, Lag3, Ngn, Sema7a, Crtam, Ccl1 and Nrn1 (FIG. 2B). Expression of 4-1BB and LAG-3 in the Egr2-GFP^{hi} CD8⁺ TILs was confirmed by flow cytometry. The majority of Egr2-GFP^{hi} cells expressed LAG-3 and/or 4-1BB. The Egr2GFP^{lo} cells also showed expression of 4-1BB and LAG-3 on a subpopulation at day 14 (FIG. 2C). This result indicates either that CD8⁺ TILs expressing Egr2 encompass only a subset of the TILs expressing LAG-3 and/or 4-1BB, or that Egr2 is transiently expressed and is subsequently downregulated after the induction of LAG-3 and 4-1BB.

Using Egr2 target genes from in vitro anergic CD4⁺ T cell clones (Zheng et al., 2013; incorporated by reference in its entirety), the Egr2-driven transcriptional program was examined in sorted 4-1BB⁻LAG-3⁻ and 4-1BB⁺LAG-3⁺ cells by qRT-PCR. Of the 43 Egr2 target genes examined, 10 showed detectably increased expression in 4-1BB⁺LAG-3⁺ population, while expression of a similar subset of genes was increased in the 4-1BB⁻LAG-3⁺ population (FIG. 2D). Collectively, these data demonstrate that Egr2 is expressed in a subpopulation of CD8⁺ TILs expressing LAG-3 and/or 4-1BB, and that a subset of known Egr2 targets was detected in these larger T cell populations as a whole.

It was next examined whether Egr2 was required for expression of LAG-3 and 4-1BB among CD8⁺ TIL in vivo. To this end Egr2^{flox/flox} X pLCK-CreERT2×ROSA-YFP mice were utilized, in which oral tamoxifen administration results in a fraction of the CD8⁺ T cells deleting Egr2 and expressing YFP (FIG. 2E). This allowed comparison of both Egr2-sufficient (YFP⁻) and Egr2-deficient (YFP⁺) CD8⁺ within the same tumor. To determine that Egr2 was in fact deleted from the YFP⁺ fraction, both YFP⁺ and YFP⁻ CD8⁺ TILs were sorted and Egr2 transcripts were measured directly ex vivo and upon ex vivo stimulation. The YFP⁺ CD8⁺ TILs expressed substantially less Egr2 transcripts compared to the YFP⁻ counterparts (FIG. 2E). To determine if Egr2 is required for 4-1BB and LAG-3 expression, CD8⁺ TILs were analyzed at day 7 and 14 after tumor inoculation and compared the YFP⁺ and YFP⁻ populations to mice not treated with tamoxifen. At day 7, the YFP⁺ fraction expressed less 4-1BB and LAG-3 compared to the YFP⁻ population and the WT CD8⁺ TILs. However, expression of 4-1BB and LAG-3 was not significantly different at day 14 (FIG. 2F). This indicates that other transcriptional regulators compensate and contribute to the expression of LAG-3 and 4-1BB, especially at later time points.

Egr3 has been shown to have overlapping function with Egr2 (Safford et al., 2005; incorporated by reference in its entirety) and HIF1α can contribute to 4-1BB expression (Palazón et al., 2012). To investigate whether these transcription factors may compensate for 4-1BB and/or LAG-3 expression we sorted Egr2GFP^{hi} and Egr2GFP^{lo} CD8⁺ TILs expressing 4-1BB and LAG-3 on day 7 and analyzed expression of Egr3 and HIF1α by qRT-PCR. Egr3 and HIF1α were indeed expressed in both the Egr2GFP^{hi} and Egr2GFP^{lo} populations. It was confirmed differential expression of Egr2 and CCL1 to between the Egr2GFP^{hi} and Egr2GFP^{lo} populations to assure sort purity (FIG. 2G). Together, these data indicate that Egr2 contributes to upregulation of 4-1BB and LAG-3 expression at early time points, but that other transcriptional regulators compensate and drive expression of LAG-3 and 4-1BB as the T cell-tumor interaction progresses.

CD8⁺4-1BB⁺LAG-3⁺ TILs are Oligoclonal and Enriched for Tumor Antigen Specificity

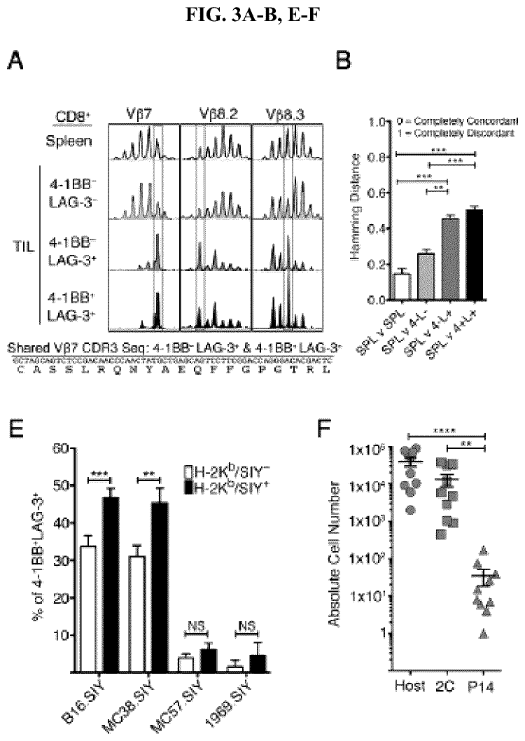

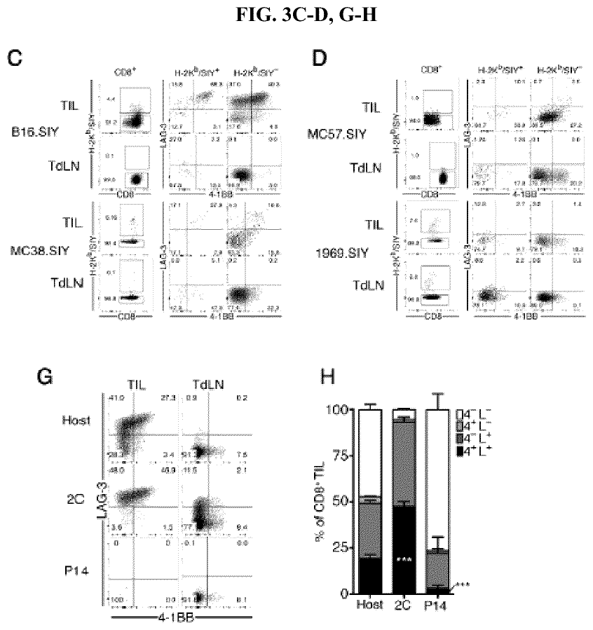

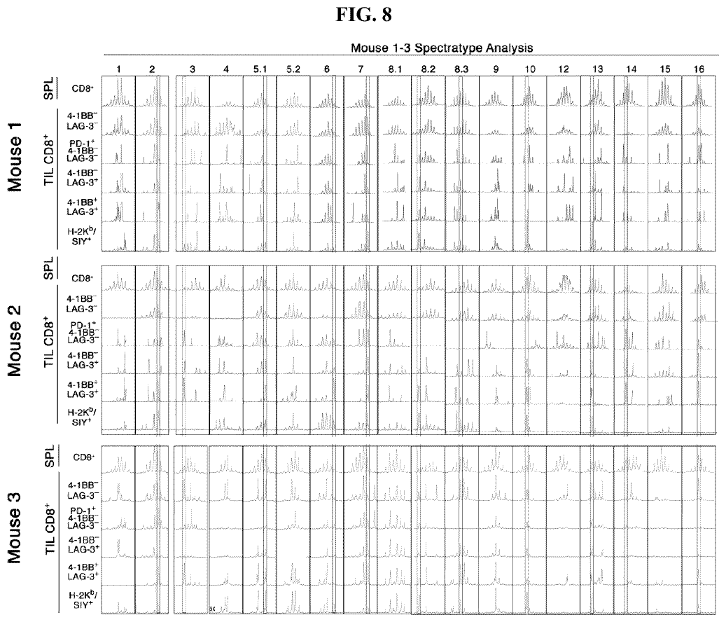

Not all T cells in the tumor microenvironment are specific for tumor-associated antigens, as memory T cells specific for irrelevant antigens are often found among TIL, and non-specific T cell trafficking has been documented in vivo (Harlin et al., 2006; incorporated by reference in its entirety). Experiments conducted during development of embodiments herein to determine whether 4-1BB⁺LAG-3⁺CD8⁺ TILs are tumor-antigen specific. LAG-3, 4-1BB and Egr2 are upregulated after TCR stimulation and experiments indicate that this population expands within the tumor microenvironment in situ. Three complementary techniques were employed. First, the CD8⁺ TILs were isolated based on LAG-3 and 4-1BB expression by cell sorting and performed TCRβ spectratype analysis. Compared to the 4-1BB⁻LAG-3⁻ TILs and CD8⁺ splenocytes, the 4-1BB⁺LAG-3⁺ TILs had a non-Gaussian distribution and shared one or two dominant peaks (FIG. 3A). Analysis of several Vβs displaying one dominant peak revealed that Vβ7 contained a single CDR3β sequence shared between the 4-1BB⁻LAG-3⁺ and 4-1BB⁺LAG-3⁺ populations, indicating a clonal relationship (FIG. 3A). To measure the oligoclonality of the CDR3β repertoires the Hamming Distance (HD) was calculated for each Vβ between the CD8⁺ TIL subpopulations and the splenic CD8⁺ population within three separate mice (FIG. 8). By transforming each spectratype into area under the curve frequency profiles the Hamming Distance computes the changes in frequency and reports a value of comparison between 0 and 1, with 0 indicating a completely identical frequency profile and 1 signifying a completely discordant profile. As a control, the HD of the splenic CD8⁺ populations between different mice was calculated (FIG. 3B, black bar). Since the splenic CD8⁺ spectratypes are largely Gaussian this value represents the HD between two similar distributions. Analysis of the HD between the CD8⁺ TIL subpopulations revealed that the 4-1BB⁺LAG-3⁺ and 4-1BB⁻LAG-3⁺ but not the 4-1BB⁻LAG-3⁻ CDR3β distributions are significantly different (less Guassian) compared to the splenic CD8⁺ population (FIG. 3B). These data indicate that the 4-1BB⁺LAG-3⁺ and 4-1BB-LAG-3⁺ populations are oligoclonal expanded subsets of TILs, indicating antigen specificity in these subpopulations.

As a second approach, the B16.SIY melanoma and MC38.SIY adenocarcinoma models were utilized. CD8⁺ T cells specific for the H-2K^{b}-restricted SIY epitope (SIYRYYGL) were monitored. SIYRYYGL/K^{b} pentamer+(H-2K^{b}/SIY) cells were found in expanded numbers within B16.SIY and MC38.SIY tumors at day 14 after tumor inoculation (FIG. 3C). Nearly 47% of the H-2K^{b}/SIY⁺ cells expressed both 4-1BB and LAG-3, in contrast to 32% of the H-2K^{b}/SIY⁻ population (FIGS. 3C and E). This enrichment of antigen-specific CD8⁺ TILs in the 4-1BB⁺LAG-3⁺ populations indicates that these markers identify tumor antigen-specific TILs. The H-2K^{b}/SIY⁻ cells also contained significant numbers of 4-1BB⁺LAG-3⁺ cells, which is consistent with the notion that tumor antigens other than SIY are also recognized by subsets of CD8⁺ TILs in vivo (FIG. 3C). H-2K^{b}/SIY⁺ cells in the spleen or TdLN did not co-express 4-1BB and LAG-3, indicating that this phenotype is acquired within the tumor microenvironment.

These features were also analyzed t in the context of tumor-antigen specific CD8⁺ TILs in two spontaneously rejected tumor models. To this end, H-2K^{b}/SIY-specific CD8⁺ TILs cells were evaluated from MC57.SIY and 1969.SIY tumors. At day 14 after tumor inoculation, approximately 5% of the H-2K^{b}/SIY-specific CD8⁺ TILs were found in the 4-1BB⁺LAG-3⁺ fraction. As with the B16.SIY tumors, no H-2K^{b}/SIY-specific CD8 T cells co-expressed 4-1BB and LAG-3 in the TdLN or spleen (not shown) (FIG. 3D). Unlike the B16.SIY and MC38.SIY tumors, no significant enrichment of 4-1BB⁺LAG-3⁺ H-2K^{b}/SIY-specific CD8⁺ TILs was observed (FIGS. 3D and E). These data indicate that tumor antigen specificity per se does not determine dysfunctionality, and that this is a feature unique to the microenvironment of progressing tumors.

As a third measure to determine if tumor-antigen specific CD8⁺ T cells acquire the 4-1BB⁺LAG-3⁺ phenotype, congenically marked 2C and P14 transgenic (Tg) T cells, isolated from 2C/Rag2^{-/-} and P14/Rag2^{-/-} mice, were transferred into tumor-bearing hosts. The 2C TCR is specific for the SIY model antigen expressed by B16.SIY tumor cells, while P14 is an irrelevant TCR specific for the LCMV-derived gp₃₃₋₄₁ epitope; both TCRs are H-2K^{b}-restricted. 2C and P14 Tg CD8⁺ T cells were transferred via tail vein 7 days after tumor inoculation. Seven days after transfer, tumors and TdLNs were extracted, and the phenotypic profile of the transferred populations was analyzed. This system allowed for the analysis of two T cell populations with defined antigen specificities within the same tumor microenvironment, as well as the polyclonal host CD8⁺ T cells. The 2C T cells were more efficiently recruited and expanded within the tumor microenvironment compared to the P14 T cells and encompassed a large fraction of the total CD8⁺ TIL population (FIG. 3F). Of the 2C T cells, nearly all expressed LAG-3 and or 4-1BB while this was true for only a small percentage of the P14 cells (FIGS. 3G and H). Consistent with the SIY-K^{b} pentamer analysis, the co-expression of LAG-3 and 4-1BB on 2C T cells was not observed in the TdLN. Together, these results demonstrate that the 4-1BB⁺LAG-3⁺ phenotype is a property of tumor antigen-specific TIL under conditions of tumor progression.

### CD8⁺ TILs Expressing LAG-3 and 4-1BB Exhibit Defective IL-2 Production Yet Produce IFN-γ and Treg-Recruiting Chemokines

Based on the characteristics of the in vitro T cell anergy model that led to the identification of Egr2 as an important regulator, experiments conducted during development of embodiments herein to determine whether the tumor-antigen specific 4-1BB⁺LAG-3⁺CD8⁺ TIL population is dysfunctional in their capacity to produce IL-2. To this end each subpopulation was sorted and stimulated with anti-CD3 and anti-CD28 mAb and analyzed IL-2 production by qRT-PCR and ELISA. Since nearly all CD8⁺ TILs displayed an activated phenotype, we used CD8⁺CD44⁺ splenocytes as a positive control. Indeed, the 4-1BB⁺LAG-3⁺ cells showed a 100-fold reduction in Il-2 mRNA and as much as a 40-fold reduction in IL-2 protein levels compared to the 4-1BB⁻LAG-3⁻ population (FIGS. 4A and 4B). As a second approach, Egr2^{hi} TIL (which are also largely 4-1BB⁺LAG-3⁺) was examined by utilizing the Egr2-GFP reporter mice. Indeed, ex vivo stimulated Egr2-GFP^{hi} CD8⁺ TILs also exhibited reduced Il-2 transcript compared to Egr2-GFP^{lo} cells (FIG. 4C). As a final approach, congenically marked 2C T cells were adoptively transferred intravenously into tumor-bearing hosts and recovered the 2C T cells 7 days later from the tumor and TdLN. 2C T cells isolated from tumors exhibited a reduced capacity to produce Il-2 transcripts, at a level equivalent to 4-1BB⁺LAG-3⁺ TILs, compared to 2C CD44⁺ T cells isolated from the TdLN (FIG. 4D). In chronic infection models, expression of PD-1 has been suggested to identify intrinsically dysfunctional or “exhausted” CD8⁺ T cells. To determine if PD-1 alone might be sufficient to identify cells that lack the capacity to produce IL-2, CD8⁺ TILs that lacked expression of LAG-3 and 4-1BB were isolated and tested for the ability of the PD-1⁺ fraction to produce IL-2. Approximately ˜10% of CD8⁺ TILs were 4-1BB-LAG-3-PD-1⁺ on day 14 and 21 (FIGS. 4E and F). Upon ex vivo stimulation, this population retained the capacity to produce 11-2 mRNA at a level comparable to the 4-1BB⁻LAG-3⁻ cells (FIG. 4G). These results indicate that PD-1 expression alone is not sufficient to identify dysfunctional TIL in the tumor microenvironment.

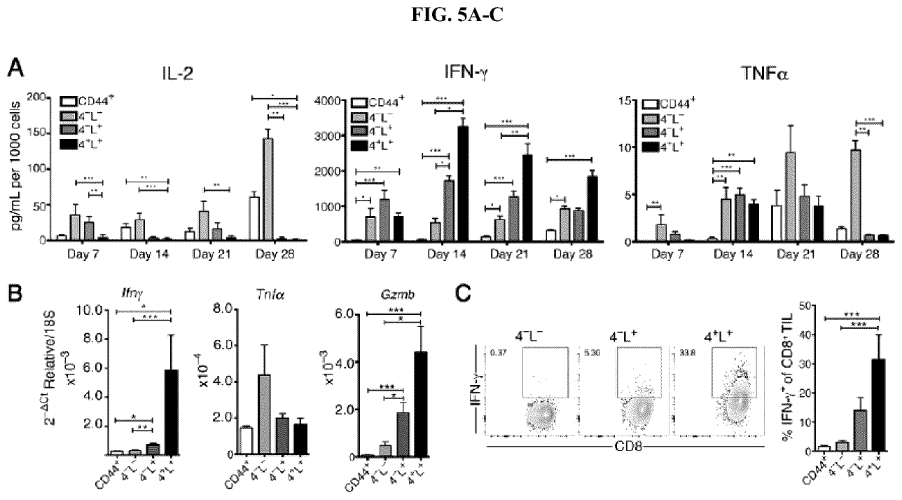

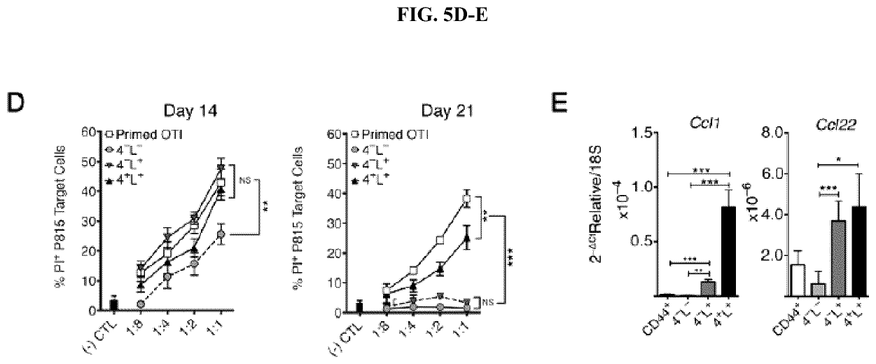

To further examine functional alterations during tumor progression we tested for protein levels of IL-2, IFN-γ and TNF-α after TCR stimulation. As the loss of the ability of CD8⁺ TILs to produce cytokines is suggested to be a temporal process reported initiated following entry into the tumor microenvironment (Waugh et al., 2016; Schietinger et al., 2016; incorporated by reference in their entireties) or progressively after 30 days in the chronic LCMV model (Wherry et al., 2007; incorporated by reference in its entirety), cytokine production was tested on day 7, 14, 21 and 28. The 4-1BB⁺LAG-3⁺ population lost the capacity to produce IL-2 as early as day 7 while the 4-1BB⁻LAG-3⁺ population lost IL-2 production between day 7 and day 14 (FIG. 5A). The 4-1BB⁻LAG-3⁻ population did not lose the ability to produce IL-2 at any time point tested (FIG. 5A), supporting the notion that this population is not tumor antigen specific and that differentiation into the dysfunctional state is an antigen-dependent process (Schietinger et al., 2016; incorporated by reference in its entirety). The 4-1BB⁺LAG-3⁺ population produced more IFN-γ at all time points after day 7 compared to their negative counterparts, albeit with a slight decrease in IFN-γ production over time. While the increase in IFN-γ was maintained until later time points, TNF-α production was lost by day 28 (FIG. 5A).

Experiments were conducted during development of embodiments herein to evaluate production of cytokines directly in the tumor without in vitro restimulation, which may more closely reflect which T cells were receiving TCR stimulation in situ. Each T cell population was sorted directly ex vivo without any culturing and mRNA levels were measured by qRT-PCR. Elevated Ifn-γ and Gzmb transcripts were observed from the 4-1BB⁺LAG-3⁺ subpopulation, along with a slight decrease in Tnf-α levels, compared to the 4-1BB⁻LAG-3⁻ cells (FIG. 5B). Production of IFN-γ in primary TILs was confirmed by injecting tumors with Brefeldin A prior to analysis by intracellular cytokine staining. Consistent with the mRNA expression, the 4-1BB⁺LAG-3⁺ population produced significantly greater amounts of IFN-γ protein (FIG. 5C). Thus, the 4-1BB⁺LAG-3⁺ TIL is not completely devoid of functionality, as they continue to produce IFN-γ despite defective production of IL-2. This phenotype is consistent with in vitro T cell anergy models (Jenkins et al., 1987; incorporated by reference in its entirety).

To test whether the 4-1BB⁺LAG-3⁺ population still retains cytotoxic capacity, re-directed lysis was performed by co-culturing anti-CD3 bound P815 mastocytoma target cells with the different CD8⁺ TIL subpopulations directly after sorting. 4-1BB⁺LAG-3⁺CD8⁺ TILs isolated from day 14 tumors were able to lyse target cells at a comparable efficacy to in vitro primed OT-I cells. 4-1BB⁺LAG-3⁺ TILs isolated from day 21 tumors were still able to lyse target cells, albeit to a lesser extent compared to primed OT-I cells (FIG. 5D).

CD8⁺ T cells in the tumor can be the source of the chemokine CCL22 that recruits FoxP3⁺ regulatory T cells (Tregs) to the tumor microenvironment (Spranger et al., 2013; incorporated by reference in its entirety). In addition, the chemokine Ccl1 was an Egr2 target in anergic T cells (Zheng et al., 2013; incorporated by reference in its entirety), and it has been suggested that CCL1 also contribute to Treg recruitment in the tumor context in vivo (Hoelzinger et al., 2010; incorporated by reference in its entirety). However, whether all CD8⁺ T cells in the tumor produce these chemokines or if they are only produced by subpopulations of T cells had not been determined. To address this the CD8⁺ TIL phenotypic subpopulations were analyzed for Ccl1 and Ccl22 mRNA expression directly ex vivo by qRT-PCR. Indeed, the 4-1BB⁺LAG-3⁺ TIL population produced substantially greater Ccl1 and Ccl22 compared to their negative counterparts or to splenic CD8⁺CD44⁺ T cells (FIG. 4K). As a control, expression of a distinct chemokine Ccl5 was found not to be differentially expressed.

Together, these data show that co-expression of 4-1BB and LAG-3 delineates tumor antigen-specific CD8⁺ TIL that lack the ability to produce IL-2 yet retain the ability to produce IFN-7, kill target cells in vitro, and secrete chemokines capable of Treg recruitment. Given the fact that IFN-7 is responsible for the upregulation of PD-L1 and IDO in the tumor microenvironment, and that chemokines produced by CD8⁺ TIL contribute to Treg recruitment (Spranger et al., 2013; incorporated by reference in its entirety), these data indicate that the 4-1BB⁺LAG-3⁺ population contributes to the network of immune suppressive mechanisms within the tumor microenvironment that limit the efficacy of anti-tumor immunity.

Gene Expression Profiling Reveals that CD8⁺4-1BB⁺LAG-3⁺ TILs Express an Extensive Array of Additional Co-Stimulatory and Co-Inhibitory Receptors

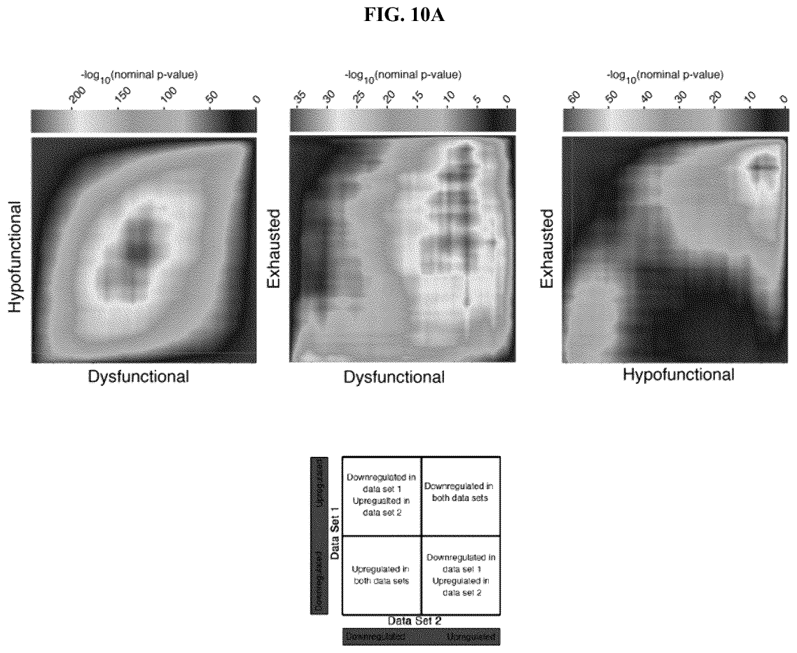

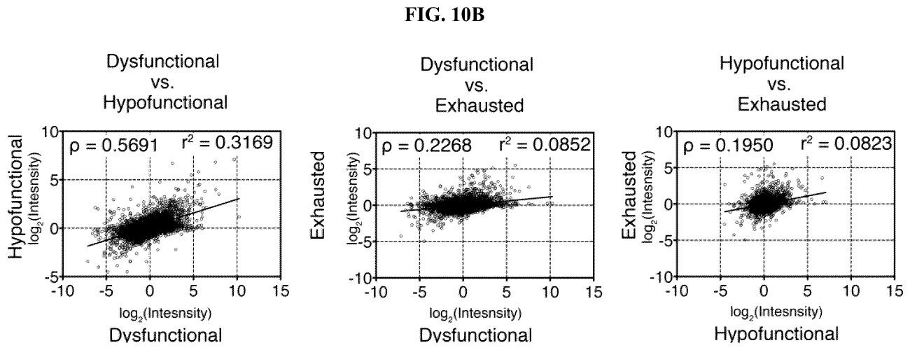

Having in hand surface markers that define tumor antigen-specific dysfunctional CD8⁺ TILs, experiments conducted during development of embodiments herein to compare the gene expression profile of this population to other published profiles of dysfunctional CD8⁺ T cells to determine genes that regulate or are differentially expressed in cells in this dysfunctional state. To this end, a cross-study comparison was conducted of the transcriptional profiles of the “dysfunctional” 4-1BB⁺LAG-3⁺ CD8⁺ TILs, “hypofunctional” CD8⁺ TILs from a study utilizing the murine CT26 tumor model (Waugh et al., 2016; incorporated by reference in its entirety) and LCMV “exhausted” GP33 specific CD8⁺ T cells (Doering et al., 2012; incorporated by reference in its entirety). The results are depicted in Table 2. Only genes with a 2-fold increase over controls from each study independently were considered. Over a 2-fold greater number of genes was found to be shared between the dysfunctional TIL dataset and the previously published hypofunctional CD8⁺ TIL data, than with the exhausted T cell profile (FIG. 6A). In addition, a rank-rank hypergeometric overlap (RRHO) analysis indicated a greater statistically significant overlap (FIG. 10A) and a greater correlation (FIG. 10B) between the current dysfunctional TIL and the published hypofunctional CD8⁺ TIL gene expression profiles compared to the virally induced exhausted CD8⁺ T cell profile, indicating a more similar molecular program between CD8⁺ T cells isolate from tumors compared to chronic viral infection.

TABLE 2
Differentially regulated genes in CD8⁺ 4-1BB⁺ LAG-3⁺ TILs

| | | Log2-Fold | Gene | | Log2-Fold |
|---|---|---|---|---|---|
| Gene | Gene Description | Change | Symbol | Gene Description | Change |
| GLDC | glycine decarboxylase | 11.25109772 | CRY2 | cryptochrome circadian | −1.546648257 |
| | | | | clock 2 | |
| GZMD | Granzyme D | 10.66720027 | KCMF1 | potassium channel | −1.546835341 |
| | | | | modulatory factor 1 | |
| SLC17A6 | solute carrier family 17 | 8.946467699 | RHOB | ras homolog family | −1.548813112 |
| | member 6 | | | member B | |
| IL1R2 | interleukin 1 receptor | 7.595353131 | KRT15 | keratin 15 | −1.549018071 |
| | type 2 | | | | |
| LTF | lactotransferrin | 7.530211233 | RRAD | RRAD, Ras related | −1.549530357 |
| | | | | glycolysis inhibitor and | |
| | | | | calcium channel regulator | |
| NRGN | neurogranin | 7.334049768 | C3 | complement component 3 | −1.549960037 |
| GZME | granzyme E | 7.160375687 | ITFG3 | Description Not Found | −1.550162812 |
| RPL6 | ribosomal protein L6 | 7.142107057 | HAAO | 3-hydroxyanthranilate 3,4- | −1.550553207 |
| | | | | dioxygenase | |
| NRN1 | neuritin 1 | 7.087993146 | RNF138 | ring finger protein 138 | −1.551449524 |
| LPL | lipoprotein lipase | 7.004501392 | UNC93B1 | unc-93 homolog B1 | −1.551767491 |
| | | | | (C. elegans) | |
| CLGN | calmegin | 6.933690655 | ANKZF1 | ankyrin repeat and zinc | −1.552214097 |
| | | | | finger domain containing 1 | |
| CD70 | CD70 molecule | 6.906890596 | IFITM3 | interferon induced | −1.552644542 |
| | | | | transmembrane protein 3 | |
| AREG | amphiregulin | 6.712870868 | TXNIP | thioredoxin interacting | −1.552785452 |
| | | | | protein | |
| ZRANB3 | zinc finger RANBP2-type | 6.595443985 | LMAN1L | lectin, mannose | −1.554588852 |
| | containing 3 | | | binding 1 like | |
| ASNS | asparagine synthetase | 6.59496878 | ALDH3B1 | aldehyde dehydrogenase 3 | −1.554711558 |
| | (glutamine-hydrolyzing) | | | family member B1 | |
| FANCD2 | Fanconi anemia | 6.353146826 | GIP | gastric inhibitory | −1.555511104 |
| | complementation | | | polypeptide | |
| | group D2 | | | | |
| GM156 | predicted gene | 6.293701542 | COX7A2L | cytochrome c oxidase | −1.555572553 |
| | 156(Gm156) | | | subunit 7A2 like | |
| ACAA1B | acetyl-Coenzyme A | 6.293701542 | APPL2 | adaptor protein, | −1.555598704 |
| | acyltransferase | | | phosphotyrosine interacting | |
| | 1B(Acaa1b) | | | with PH domain and | |
| | | | | leucine zipper 2 | |
| IGF2BP3 | insulin like growth factor | 6.186857067 | KLHL22 | kelch like family | −1.555929583 |
| | 2 mRNA binding | | | member 22 | |
| | protein 3 | | | | |
| GZMG | granzyme G | 6.093813673 | OLFR272 | olfactory receptor | −1.557482156 |
| | | | | 272(Olfr272) | |
| CIB2 | calcium and integrin | 6.007868243 | LRRC29 | leucine rich repeat | −1.559366716 |
| | binding family member 2 | | | containing 29 | |
| ATG9B | autophagy related 9B | 5.986410935 | A630095E13RIK | Description Not Found | −1.560714954 |
| XKR8 | XK related 8 | 5.977279924 | OLFR194 | olfactory receptor | −1.560714954 |
| | | | | 194(Olfr194) | |
| EPDR1 | ependymin related 1 | 5.956521363 | OLFR1013 | olfactory receptor | −1.560714954 |
| | | | | 1013(Olfr1013) | |
| SPP1 | secreted phosphoprotein 1 | 5.797769502 | GLRA4 | glycine receptor alpha 4 | −1.560714954 |
| RGS8 | regulator of G-protein | 5.753805672 | P2RY6 | pyrimidinergic receptor | −1.560714954 |
| | signaling 8 | | | P2Y6 | |
| MDFIC | MyoD family inhibitor | 5.730639956 | RASGEF1B | RasGEF domain family | −1.560714954 |
| | domain containing | | | member 1B | |
| DMWD | dystrophia myotonica, | 5.687200695 | IL22RA2 | interleukin 22 receptor | −1.560714954 |
| | WD repeat containing | | | subunit alpha 2 | |
| KIF11 | kinesin family member 11 | 5.669593751 | LIN7C | lin-7 homolog C, crumbs | −1.560714954 |
| | | | | cell polarity complex | |
| | | | | component | |
| LGI2 | leucine rich repeat LGI | 5.655351829 | DMRT1 | doublesex and mab-3 | −1.560714954 |
| | family member 2 | | | related transcription factor 1 | |
| ZFP41 | ZFP41 zinc finger protein | 5.615445725 | TSPAN12 | tetraspanin 12 | −1.560714954 |
| MLKL | mixed lineage kinase | 5.605849867 | PAK3 | p21 (RAC1) activated | −1.560714954 |
| | domain-like | | | kinase 3 | |
| CENPH | centromere protein H | 5.563768278 | COL2A1 | collagen type II alpha 1 | −1.560714954 |
| | | | | chain | |
| SERPINF1 | serpin family F member 1 | 5.5360529 | SLC37A1 | solute carrier family 37 | −1.560714954 |
| | | | | member 1 | |
| UNC13B | unc-13 homolog B | 5.503030646 | PSD3 | pleckstrin and Sec7 domain | −1.560714954 |
| | (C. elegans) | | | containing 3 | |
| MLANA | melan-A | 5.496654083 | RDH5 | retinol dehydrogenase 5 | −1.560714954 |
| PES1 | pescadillo ribosomal | 5.484376709 | ABCA3 | ATP binding cassette | −1.561263453 |
| | biogenesis factor 1 | | | subfamily A member 3 | |
| 2900026A02RIK | Description Not Found | 5.477353527 | PLA2G4E | phospholipase A2 group | −1.561650879 |
| | | | | IVE | |
| OSR2 | odd-skipped related | 5.416164165 | DDIT3 | DNA damage inducible | −1.563566526 |
| | transciption factor 2 | | | transcript 3 | |
| MPP6 | membrane palmitoylated | 5.408506442 | ZFP12 | zinc finger protein | −1.564308646 |
| | protein 6 | | | 12(Zfp12) | |
| HIST1H3C | histone cluster 1, H3c | 5.397460726 | PIGYL | phosphatidylinositol glycan | −1.564585219 |
| | | | | anchor biosynthesis, class | |
| | | | | Y-like(Pigyl) | |
| PI4K2B | phosphatidylinositol 4- | 5.375039431 | CCDC97 | coiled-coil domain | −1.565355117 |
| | kinase type 2 beta | | | containing 97 | |
| SH3YL1 | SH3 and SYLF domain | 5.375039431 | OLFR1112 | olfactory receptor | −1.56589319 |
| | containing 1 | | | 1112(Olfr1112) | |
| RAD51 | RAD51 recombinase | 5.371558863 | ACTN2 | actinin alpha 2 | −1.566931646 |
| ZBTB32 | zinc finger and BTB | 5.318316841 | POLG | polymerase (DNA) gamma, | −1.567265595 |
| | domain containing 32 | | | catalytic subunit | |
| MSC | musculin | 5.285402219 | FBXO32 | F-box protein 32 | −1.567281905 |
| TG | thyroglobulin | 5.259272487 | MRPL15 | mitochondrial ribosomal | −1.570722678 |
| | | | | protein L15 | |
| RSPH1 | radial spoke head 1 | 5.236492618 | FCHSD2 | FCH and double SH3 | −1.571821211 |
| | homolog | | | domains 2 | |
| ARL11 | ADP ribosylation factor | 5.21916852 | RECQL | RecQ like helicase | −1.572889668 |
| | like GTPase 11 | | | | |
| NUDT11 | nudix hydrolase 11 | 5.215290306 | NDUFB11 | NADH:ubiquinone | −1.572889668 |
| | | | | oxidoreductase subunit B11 | |
| APBB1 | amyloid beta precursor | 5.197708158 | SOX8 | SRY-box 8 | −1.573341535 |
| | protein binding family B | | | | |
| | member 1 | | | | |
| SPINK2 | serine peptidase inhibitor, | 5.189824559 | 1700030J22RIK | Description Not Found | −1.57662394 |
| | Kazal type 2 | | | | |
| HMGN3 | high mobility group | 5.168922782 | EMB | embigin | −1.577890585 |
| | nucleosomal binding | | | | |
| | domain 3 | | | | |
| FAM20B | family with sequence | 5.12722055 | CELSR1 | cadherin EGF LAG seven- | −1.578201987 |
| | similarity 20 member B | | | pass G-type receptor 1 | |
| CDC25C | cell division cycle 25C | 5.11997861 | COL1A2 | collagen type I alpha 2 | −1.580682782 |
| | | | | chain | |
| FAM20A | family with sequence | 5.108524457 | 1700080E11RIK | Description Not Found | −1.581046002 |
| | similarity 20 member A | | | | |
| PPP1R16B | protein phosphatase 1 | 5.09592442 | GALNT12 | polypeptide N- | −1.581363645 |
| | regulatory subunit 16B | | | acetylgalactosaminyl- | |
| | | | | transferase 12 | |
| SBNO1 | strawberry notch homolog | 5.050936965 | RMND5B | required for meiotic nuclear | −1.583960816 |
| | 1 (Drosophila) | | | division 5 homolog B | |
| ST14 | suppression of | 5.026800059 | LRRC28 | leucine rich repeat | −1.583987499 |
| | tumorigenicity 14 | | | containing 28 | |
| LRRC49 | leucine rich repeat | 5.024704311 | OLFR622 | olfactory receptor | −1.584962501 |
| | containing 49 | | | 622(Olfr622) | |
| TIAM1 | T-cell lymphoma invasion | 5.004501392 | OLFR339 | olfactory receptor | −1.584962501 |
| | and metastasis 1 | | | 339(Olfr339) | |
| APLF | aprataxin and PNKP like | 4.951867504 | NEIL3 | nei like DNA glycosylase 3 | −1.584962501 |
| | factor | | | | |
| PGPEP1 | pyroglutamyl-peptidase I | 4.927185358 | SNX24 | sorting nexin 24 | −1.584962501 |
| ALCAM | activated leukocyte cell | 4.909293086 | SLC7A11 | solute carrier family 7 | −1.584962501 |
| | adhesion molecule | | | member 11 | |
| B9D1 | B9 domain containing 1 | 4.906890596 | FOXJ1 | forkhead box J1 | −1.584962501 |
| SCIN | scinderin | 4.87282876 | TAF3 | TATA-box binding protein | −1.584962501 |
| | | | | associated factor 3 | |
| EXOC3L | exocyst complex | 4.844013973 | MATN2 | matrilin 2 | −1.584962501 |
| | component 3-like(Exoc31) | | | | |
| SLC35D3 | solute carrier family 35 | 4.840463234 | ADHFE1 | alcohol dehydrogenase, iron | −1.586280668 |
| | member D3 | | | containing 1 | |
| ALDOC | aldolase, fructose- | 4.832890014 | NANOS1 | nanos C2HC-type zinc | −1.586914831 |
| | bisphosphate C | | | finger 1 | |
| TMEM205 | transmembrane protein | 4.830182468 | PPP2R5B | protein phosphatase 2 | −1.586914831 |
| | 205 | | | regulatory subunit B′beta | |
| PLEKHA8 | pleckstrin homology | 4.820178962 | USP22 | ubiquitin specific peptidase | −1.588703598 |
| | domain containing A8 | | | 22 | |
| SPC25 | SPC25, NDC80 | 4.817623258 | DAGLB | diacylglycerol lipase beta | −1.588817933 |
| | kinetochore complex | | | | |
| | component | | | | |
| PCYT1B | phosphate | 4.749534268 | KCTD6 | potassium channel | −1.589690033 |
| | cytidylyltransferase 1, | | | tetramerization domain | |
| | choline, beta | | | containing 6 | |
| SLC6A8 | solute carrier family 6 | 4.749534268 | ACTL6B | actin like 6B | −1.591351555 |
| | member 8 | | | | |
| TUBB6 | tubulin beta 6 class V | 4.749241128 | FAM129B | family with sequence | −1.5915039 |
| | | | | similarity 129 member B | |
| BSPRY | B-box and SPRY domain | 4.711494907 | APOE | apolipoprotein E | −1.591683393 |
| | containing | | | | |
| ICA1 | islet cell autoantigen 1 | 4.708739041 | GPR18 | G protein-coupled receptor | −1.592384168 |
| | | | | 18 | |
| TNFSF13B | tumor necrosis factor | 4.703211467 | GSTP2 | glutathione S-transferase, pi | −1.592559885 |
| | superfamily member 13b | | | 2(Gstp2) | |
| GSTCD | glutathione S-transferase | 4.700439718 | GPR114 | Description Not Found | −1.593829527 |
| | C-terminal domain | | | | |
| | containing | | | | |
| CCNB1 | cyclin B1 | 4.699051844 | CHUK | conserved helix-loop-helix | −1.594823937 |
| | | | | ubiquitous kinase | |
| 4930539E08RIK | Description Not Found | 4.693211287 | TAS1R3 | taste 1 receptor member 3 | −1.596595048 |
| SRXN1 | sulfiredoxin 1 | 4.66106548 | SLC7A7 | solute carrier family 7 | −1.596935142 |
| | | | | member 7 | |
| SERF1 | small EDRK-rich factor | 4.632268216 | SPIB | Spi-B transcription factor | −1.597677703 |
| | 1(Serf1) | | | | |
| CCDC77 | coiled-coil domain | 4.62935662 | POLR3A | polymerase (RNA) III | −1.599588488 |
| | containing 77 | | | subunit A | |
| RHBDF1 | rhomboid 5 homolog 1 | 4.626439137 | OLFR952 | olfactory receptor | −1.599679175 |
| | | | | 952(Olfr952) | |
| REEP3 | receptor accessory protein | 4.599912842 | 1700021F05RIK | Description Not Found | −1.601623253 |
| | 3 | | | | |
| ITGA3 | integrin subunit alpha 3 | 4.590961241 | CCDC79 | Description Not Found | −1.602195565 |
| SCCPDH | saccharopine | 4.590961241 | FAM134B | family with sequence | −1.602715966 |
| | dehydrogenase (putative) | | | similarity 134 member B | |
| MYADM | myeloid associated | 4.587964989 | SEMA3B | semaphorin 3B | −1.602884409 |
| | differentiation marker | | | | |
| FAM132A | family with sequence | 4.581953751 | FA2H | fatty acid 2-hydroxylase | −1.604494406 |
| | similarity 132 member A | | | | |
| FOXRED2 | FAD dependent | 4.572889668 | ULK1 | unc-51 like autophagy | −1.604653903 |
| | oxidoreductase domain | | | activating kinase 1 | |
| | containing 2 | | | | |
| CENPK | centromere protein K | 4.569855608 | MCOLN1 | mucolipin 1 | −1.606242992 |
| DCXR | dicarbonyl and L-xylulose | 4.562242424 | BMP5 | bone morphogenetic protein | −1.606760033 |
| | reductase | | | 5 | |
| TSPAN6 | tetraspanin 6 | 4.54225805 | ANKRD50 | ankyrin repeat domain 50 | −1.607137028 |
| UPP1 | uridine phosphorylase 1 | 4.53838296 | OLFR560 | olfactory receptor | −1.608809243 |
| | | | | 560(Olfr560) | |
| DOK4 | docking protein 4 | 4.520422249 | OLFR366 | olfactory receptor | −1.608809243 |
| | | | | 366(Olfr366) | |
| ELOVL4 | ELOVL fatty acid | 4.501439145 | OLFR273 | olfactory receptor | −1.608809243 |
| | elongase 4 | | | 273(Olfr273) | |
| KNDC1 | kinase non-catalytic C- | 4.499790117 | FHIT | fragile histidine triad | −1.608809243 |
| | lobe domain containing 1 | | | | |
| KRT17 | keratin 17 | 4.491853096 | AQP11 | aquaporin 11 | −1.608809243 |
| CHST2 | carbohydrate | 4.487315031 | TMEM176A | transmembrane protein | −1.608809243 |
| | sulfotransferase 2 | | | 176A | |
| TPX2 | TPX2, microtubule | 4.475733431 | ENAH | enabled homolog | −1.608809243 |
| | nucleation factor | | | (Drosophila) | |
| DUSP14 | dual specificity | 4.456149035 | CLDN6 | claudin 6 | −1.608809243 |
| | phosphatase 14 | | | | |
| BGN | biglycan | 4.449561375 | SP1 | Sp1 transcription factor | −1.608809243 |
| FKBP9 | FK506 binding protein 9 | 4.442943496 | SP140 | SP140 nuclear body protein | −1.608809243 |
| CAPN5 | calpain 5 | 4.385431037 | RASGRP3 | RAS guanyl releasing | −1.608809243 |
| | | | | protein 3 | |
| SLC1A4 | solute carrier family 1 | 4.375039431 | HIF3A | hypoxia inducible factor 3 | −1.609422664 |
| | member 4 | | | alpha subunit | |
| IDI2 | isopentenyl-diphosphate | 4.357552005 | FYCO1 | FYVE and coiled-coil | −1.611220598 |
| | delta isomerase 2 | | | domain containing 1 | |
| AKR1E1 | aldo-keto reductase | 4.346596388 | FBXL12 | F-box and leucine rich | −1.6119368 |
| | family 1, member | | | repeat protein 12 | |
| | E1(Akrle1) | | | | |
| GNB4 | G protein subunit beta 4 | 4.336088936 | KLRA10 | killer cell lectin-like | −1.618484777 |
| | | | | receptor subfamily A, | |
| | | | | member 10(Klra10) | |
| CPNE2 | copine 2 | 4.318640898 | ABAT | 4-aminobutyrate | −1.62058641 |
| | | | | aminotransferase | |
| FAM132B | family with sequence | 4.259272487 | AMHR2 | anti-Mullerian hormone | −1.62058641 |
| | similarity 132, member | | | receptor type 2 | |
| | B(Fam132b) | | | | |
| SLC6A12 | solute carrier family 6 | 4.259272487 | DDX3Y | DEAD-box helicase 3, | −1.620649859 |
| | member 12 | | | Y-linked | |
| CPLX1 | complexin 1 | 4.240314329 | LGALS4 | galectin 4 | −1.621550215 |
| PDCD1 | programmed cell death 1 | 4.221103725 | SPG20 | spastic paraplegia 20 | −1.621653602 |
| | | | | (Troyer syndrome) | |
| UTF1 | undifferentiated | 4.201633861 | CTRL | chymotrypsin like | −1.62729369 |
| | embryonic cell | | | | |
| | transcription factor 1 | | | | |
| WDR60 | WD repeat domain 60 | 4.14974712 | GREM2 | gremlin 2, DAN family | −1.627927342 |
| | | | | BMP antagonist | |
| EGFL7 | EGF like domain multiple | 4.137503524 | ZMAT3 | zinc finger matrin-type 3 | −1.628362075 |
| | 7 | | | | |
| ASPM | abnormal spindle | 4.133399125 | AP4M1 | adaptor related protein | −1.628898157 |
| | microtubule assembly | | | complex 4 mu 1 subunit | |
| TMBIM1 | transmembrane BAX | 4.104628811 | NT5C2 | 5′-nucleotidase, cytosolic II | −1.63059747 |
| | inhibitor motif containing | | | | |
| | 1 | | | | |
| KNTC1 | kinetochore associated 1 | 4.093952772 | TMIE | transmembrane inner ear | −1.631606148 |
| 1700019D03RIK | Description Not Found | 4.087462841 | OLFR556 | olfactory receptor | −1.632268216 |
| | | | | 556(Olfr556) | |
| TM4SF5 | transmembrane 4 L six | 4.087462841 | OLFR463 | olfactory receptor | −1.632268216 |
| | family member 5 | | | 463(Olfr463) | |
| BIRC5 | baculoviral IAP repeat | 4.027905997 | CTS3 | cathepsin 3(Cts3) | −1.632268216 |
| | containing 5 | | | | |
| SYNGR3 | synaptogyrin 3 | 4.022367813 | OAS1B | 2′-5′ oligoadenylate | 1.632268216 |
| | | | | synthetase 1B(Oas1b) | |
| PLSCR4 | phospholipid scramblase | 4 | KCNF1 | potassium voltage-gated | −1.632268216 |
| | 4 | | | channel modifier subfamily | |
| | | | | F member 1 | |
| KIF15 | kinesin family member 15 | 3.962376898 | GCGR | glucagon receptor | −1.632268216 |
| TICAM2 | toll like receptor adaptor | 3.958842675 | NR1I3 | nuclear receptor subfamily | −1.632268216 |
| | molecule 2 | | | 1 group I member 3 | |
| CENPM | centromere protein M | 3.957682486 | FSTL1 | follistatin like 1 | −1.632268216 |
| KIF4 | kinesin family member | 3.956097191 | ASAP3 | ArfGAP with SH3 domain, | −1.632268216 |
| | 4(Kif4) | | | ankyrin repeat and PH | |
| | | | | domain 3 | |
| E2F2 | E2F transcription factor 2 | 3.93191939 | IHH | indian hedgehog | −1.632268216 |
| MSN | moesin | 3.930737338 | SEMA3A | semaphorin 3A | −1.632268216 |
| PTPRA | protein tyrosine | 3.928989949 | RAMP1 | receptor activity modifying | −1.632575446 |
| | phosphatase, receptor | | | protein 1 | |
| | type A | | | | |
| BC026585 | cDNA sequence | 3.882643049 | NFKBID | NFKB inhibitor delta | −1.633158642 |
| | BC026585(BC026585) | | | | |
| IQGAP3 | IQ motif containing | 3.867896464 | KLK15 | kallikrein related peptidase | −1.633773522 |
| | GTPase activating protein | | | 15 | |
| | 3 | | | | |
| CD244 | CD244 molecule | 3.867896464 | CYP1B1 | cytochrome P450 family 1 | −1.634684534 |
| | | | | subfamily B member 1 | |
| HIST1H3G | histone cluster 1, H3g | 3.837943242 | DNAJA1 | DnaJ heat shock protein | −1.635111002 |
| | | | | family (Hsp40) member A1 | |
| SLC15A3 | solute carrier family 15 | 3.832890014 | SDSL | serine dehydratase like | −1.635807742 |
| | member 3 | | | | |
| GIPC2 | GIPC PDZ domain | 3.817623258 | CCDC137 | coiled-coil domain | −1.636838653 |
| | containing family | | | containing 137 | |
| | member 2 | | | | |
| UTP15 | UTP15, small subunit | 3.812498225 | ZSWIM4 | zinc finger SWIM-type | −1.638152805 |
| | processome component | | | containing 4 | |
| PDIA6 | protein disulfide | 3.812498225 | BBC3 | BCL2 binding component 3 | −1.638336813 |
| | isomerase family A | | | | |
| | member 6 | | | | |
| JDP2 | Jun dimerization protein 2 | 3.807354922 | SOCS3 | suppressor of cytokine | −1.638876738 |
| | | | | signaling 3 | |
| MESDC1 | mesoderm development | 3.806723946 | 2900092C05RIK | Description Not Found | −1.639157339 |
| | candidate 1 | | | | |
| GAS2 | growth arrest specific 2 | 3.802193217 | CSRNP2 | cysteine and serine rich | −1.639383642 |
| | | | | nuclear protein 2 | |
| IL4I1 | interleukin 4 induced 1 | 3.802193217 | BLOC1S3 | biogenesis of lysosomal | −1.639585785 |
| | | | | organelles complex 1 | |
| | | | | subunit 3 | |
| PHF19 | PHD finger protein 19 | 3.802193217 | ELL | elongation factor for RNA | −1.64021945 |
| | | | | polymerase II | |
| CKAP2L | cytoskeleton associated | 3.797012978 | GTF3C4 | general transcription factor | −1.640658029 |
| | protein 2 like | | | IIIC subunit 4 | |
| GSTT1 | glutathione S-transferase | 3.791814071 | MYLPF | myosin light chain, | −1.640660074 |
| | theta 1 | | | phosphorylatable, fast | |
| | | | | skeletal muscle | |
| ADAM3 | a disintegrin and | 3.781359714 | CYP2A12 | cytochrome P450, family 2, | −1.641947141 |
| | metallopeptidase domain | | | subfamily a, polypeptide | |
| | 3 (cyritestin)(Adam3) | | | 12(Cyp2a12) | |
| SLAMF7 | SLAM family member 7 | 3.781359714 | RNF139 | ring finger protein 139 | −1.642010395 |
| MCPT8 | mast cell protease | 3.770829046 | C78339 | Description Not Found | −1.643573868 |
| | 8(Mcpt8) | | | | |
| DGKG | diacylglycerol kinase | 3.765534746 | EDEM1 | ER degradation enhancing | −1.64385619 |
| | gamma | | | alpha-mannosidase like | |
| | | | | protein 1 | |
| NLGN2 | neuroligin 2 | 3.716990894 | UBE2E1 | ubiquitin conjugating | −1.645859791 |
| | | | | enzyme E2 E1 | |
| SERPINE2 | serpin family E member 2 | 3.694880193 | PALMD | palmdelphin | −1.646322067 |
| IL10 | interleukin 10 | 3.689299161 | AMICA1 | adhesion molecule, interacts | −1.647478619 |
| | | | | with CXADR antigen | |
| | | | | 1(Amica1) | |
| SLC6A13 | solute carrier family 6 | 3.689299161 | KLHL11 | kelch like family member | −1.650611828 |
| | member 13 | | | 11 | |
| STAU2 | staufen double-stranded | 3.666756592 | IFNGR2 | interferon gamma receptor | −1.651050175 |
| | RNA binding protein 2 | | | 2 (interferon gamma | |
| | | | | transducer 1) | |
| ARHGDIG | Rho GDP dissociation | 3.655351829 | DECR1 | 2,4-dienoyl-CoA reductase | −1.651406438 |
| | inhibitor gamma | | | 1, mitochondrial | |
| TK1 | thymidine kinase 1 | 3.637477097 | SAMD3 | sterile alpha motif domain | −1.653213853 |
| | | | | containing 3 | |
| PCYT1A | phosphate | 3.617728231 | 9130409123RIK | Description Not Found | −1.655351829 |
| | cytidylyltransferase 1, | | | | |
| | choline, alpha | | | | |
| LAMB3 | laminin subunit beta 3 | 3.608809243 | 2010107G12RIK | Description Not Found | −1.655351829 |
| UBE2N | ubiquitin conjugating | 3.590961241 | ZFP354B | zinc finger protein | −1.655351829 |
| | enzyme E2 N | | | 354B(Zfp354b) | |
| STARD8 | StAR related lipid transfer | 3.578938713 | TAS2R143 | taste receptor, type 2, | −1.655351829 |
| | domain containing 8 | | | member 143(Tas2r143) | |
| PRR5 | proline rich 5 | 3.578938713 | OLFR65 | olfactory receptor | −1.655351829 |
| | | | | 65(Olfr65) | |
| BDH2 | 3-hydroxybutyrate | 3.554588852 | NRP | neural regeneration | −1.655351829 |
| | dehydrogenase, type 2 | | | protein(Nrp) | |
| FAM124B | family with sequence | 3.548436625 | DOK3 | docking protein 3 | −1.655351829 |
| | similarity 124 member B | | | | |
| MGAT3 | mannosyl (beta-1,4-)- | 3.548436625 | HIGD1A | HIG1 hypoxia inducible | −1.655351829 |
| | glycoprotein beta-1,4-N- | | | domain family member 1A | |
| | acetylglucosaminyl- | | | | |
| | transferase | | | | |
| LAG3 | lymphocyte activating 3 | 3.542346309 | CCDC13 | coiled-coil domain | −1.655351829 |
| | | | | containing 13 | |
| GDPD5 | glycerophosphodiester | 3.538812733 | ANGPTL2 | angiopoietin like 2 | −1.655351829 |
| | phosphodiesterase domain | | | | |
| | containing 5 | | | | |
| RNF168 | ring finger protein 168 | 3.5360529 | CNGB3 | cyclic nucleotide gated | −1.655351829 |
| | | | | channel beta 3 | |
| LYPLA1 | lysophospholipase I | 3.529820947 | HOXD4 | homeobox D4 | −1.655351829 |
| TUBGCP4 | tubulin gamma complex | 3.523561956 | KIFC3 | kinesin family member C3 | −1.655351829 |
| | associated protein 4 | | | | |
| PYGL | phosphorylase, glycogen, | 3.51412226 | AMACR | alpha-methylacyl-CoA | −1.655351829 |
| | liver | | | racemase | |
| CCL3 | C-C motif chemokine | 3.510281539 | 2310014L17RIK | Description Not Found | −1.655707015 |
| | ligand 3 | | | | |
| BCAT1 | branched chain amino | 3.508163667 | BRAP | BRCA1 associated protein | −1.657090723 |
| | acid transaminase 1 | | | | |
| ATP6V0A1 | ATPase H+ transporting | 3.501439145 | SLC39A1 | solute carrier family 39 | −1.657631089 |
| | V0 subunit a1 | | | member 1 | |
| EIF4E | eukaryotic translation | 3.498250868 | OLFR419 | olfactory receptor | −1.65813796 |
| | initiation factor 4E | | | 419(Olfr419) | |
| HIST1H4B | histone cluster 1, H4b | 3.491853096 | NHP2L1 | NHP2 non-histone | −1.658298045 |
| | | | | chromosome protein 2-like | |
| | | | | 1 (S. cerevisiae)(Nhp211) | |
| LAD1 | ladinin 1 | 3.49085426 | STOML2 | stomatin like 2 | −1.659357735 |
| ITGAV | integrin subunit alpha V | 3.485426827 | SAMM50 | SAMM50 sorting and | −1.662400762 |
| | | | | assembly machinery | |
| | | | | component | |
| MRPL47 | mitochondrial ribosomal | 3.485426827 | CCDC91 | coiled-coil domain | −1.6632299 |
| | protein L47 | | | containing 91 | |
| CAMK2N1 | calcium/calmodulin | 3.484460783 | ATF3 | activating transcription | −1.663483642 |
| | dependent protein kinase | | | factor 3 | |
| | II inhibitor 1 | | | | |
| UEVLD | UEV and lactate/malate | 3.465974465 | RAI1 | retinoic acid induced 1 | −1.663885989 |
| | dehyrogenase domains | | | | |
| SFXN4 | sideroflexin 4 | 3.462706751 | RRAS2 | related RAS viral (r-ras) | −1.665826896 |
| | | | | oncogene homolog 2 | |
| 2810417H13RIK | Description Not Found | 3.461634298 | UROS | uroporphyrinogen III | −1.665923156 |
| | | | | synthase | |
| RAD51AP1 | RAD51 associated protein | 3.459431619 | SCOC | short coiled-coil protein | −1.666272349 |
| FUT4 | fucosyltransferase 4 | 3.452858965 | DUSP10 | dual specificity phosphatase | −1.666485948 |
| | | | | 10 | |
| CTNNBIP1 | catenin beta interacting | 3.44625623 | CYB5R4 | cytochrome b5 reductase 4 | −1.666756592 |
| | protein 1 | | | | |
| ZBTB8OS | zinc finger and BTB | 3.426264755 | 9930104L06RIK | Description Not Found | −1.667150978 |
| | domain containing 8 | | | | |
| | opposite strand | | | | |
| LYSMD4 | LysM domain containing | 3.42259008 | ZFP579 | zinc finger protein | −1.669023741 |
| | 4 | | | 579(Zfp579) | |
| DIAP3 | Description Not Found | 3.40599236 | RGP1 | RGP1 homolog, RAB6A | −1.669393721 |
| | | | | GEF complex partner 1 | |
| PTGIS | prostaglandin I2 | 3.399171094 | PIAS2 | protein inhibitor of | −1.672137196 |
| | (prostacyclin) synthase | | | activated STAT 2 | |
| MOAP1 | modulator of apoptosis 1 | 3.392317423 | METTL1 | methyltransferase like 1 | −1.672425342 |
| SLC27A3 | solute carrier family 27 | 3.392317423 | POU5F1 | POU class 5 homeobox 1 | −1.673854965 |
| | member 3 | | | | |
| MRPL39 | mitochondrial ribosomal | 3.371492175 | SERPINB6C | serine (or cysteine) | −1.673932658 |
| | protein L39 | | | peptidase inhibitor, clade B, | |
| | | | | member 6c(Serpinb6c) | |
| WTAP | Wilms tumor 1 associated | 3.364572432 | STXBP4 | syntaxin binding protein 4 | −1.675552278 |
| | protein | | | | |
| RAD54L | RAD54-like | 3.356589854 | RIMS3 | regulating synaptic | −1.676120648 |
| | (S. cerevisiae) | | | membrane exocytosis 3 | |
| CETN4 | centrin 4(Cetn4) | 3.336283388 | XYLT2 | xylosyltransferase 2 | −1.676976793 |
| CEP55 | centrosomal protein 55 | 3.329123596 | TAS2R107 | taste receptor, type 2, | −1.678071905 |
| | | | | member 107(Tas2r107) | |
| CYP4F39 | cytochrome P450, family | 3.321928095 | SKP1A | S-phase kinase-associated | −1.678071905 |
| | 4, subfamily f, | | | protein 1A(Skp1a) | |
| | polypeptide 39(Cyp4f39) | | | | |
| PTPN5 | protein tyrosine | 3.314696526 | OLFR165 | olfactory receptor | −1.678071905 |
| | phosphatase, non-receptor | | | 165(Olfr165) | |
| | type 5 | | | | |
| TUBE1 | tubulin epsilon 1 | 3.292781749 | OLFR111 | olfactory receptor | −1.678071905 |
| | | | | 111(Olfr111) | |
| TCAM1 | testicular cell adhesion | 3.285402219 | CYP4A12A | cytochrome P450, family 4, | −1.678071905 |
| | molecule 1(Tcam1) | | | subfamily a, polypeptide | |
| | | | | 12a(Cyp4a12a) | |
| MID1IP1 | MID1 interacting protein | 3.263034406 | TLR6 | toll like receptor 6 | −1.678071905 |
| | 1 | | | | |
| ABHD6 | abhydrolase domain | 3.260682276 | KCNS3 | potassium voltage-gated | −1.678071905 |
| | containing 6 | | | channel modifier subfamily | |
| | | | | S member 3 | |
| ZCCHC4 | zinc finger CCHC-type | 3.255500733 | FARSA | phenylalanyl-tRNA | −1.678071905 |
| | containing 4 | | | synthetase alpha subunit | |
| MGST3 | microsomal glutathione | 3.25353624 | SLC2A4 | solute carrier family 2 | −1.678071905 |
| | S-transferase 3 | | | member 4 | |
| BC022687 | cDNA sequence | 3.247927513 | GDPD4 | glycerophosphodiester | −1.678071905 |
| | BC022687(BC022687) | | | phosphodiesterase domain | |
| | | | | containing 4 | |
| ACSF3 | acyl-CoA synthetase | 3.24325855 | RCAN1 | regulator of calcineurin 1 | −1.678071905 |
| | family member 3 | | | | |
| ADAM8 | ADAM metallopeptidase | 3.240314329 | CCDC82 | coiled-coil domain | −1.678071905 |
| | domain 8 | | | containing 82 | |
| SGCB | sarcoglycan beta | 3.237034772 | CDYL2 | chromodomain protein, Y- | −1.678071905 |
| | | | | like 2 | |
| SOCS2 | suppressor of cytokine | 3.232660757 | MBD5 | methyl-CpG binding | −1.678071905 |
| | signaling 2 | | | domain protein 5 | |
| HIST1H2AG | histone cluster 1, H2ag | 3.223000387 | ACSL1 | acyl-CoA synthetase long- | −1.678071905 |
| | | | | chain family member 1 | |
| CRMP1 | collapsin response | 3.201633861 | OTUB2 | OTU deubiquitinase, | −1.678071905 |
| | mediator protein 1 | | | ubiquitin aldehyde binding | |
| | | | | 2 | |
| RPS19BP1 | ribosomal protein S19 | 3.201633861 | NPPA | natriuretic peptide A | −1.678071905 |
| | binding protein 1 | | | | |
| 1700020L24RIK | Description Not Found | 3.193771743 | LY96 | lymphocyte antigen 96 | −1.679594789 |
| CCDC109B | coiled-coil domain | 3.181276986 | OLFR351 | olfactory receptor | −1.680730557 |
| | containing | | | 351(Olfr351) | |
| | 109B(Ccdc109b) | | | | |
| UBE2C | ubiquitin conjugating | 3.177917792 | TGFBR1 | transforming growth factor | −1.681068055 |
| | enzyme E2 C | | | beta receptor 1 | |
| SLC25A16 | solute carrier family 25 | 3.177917792 | KLHL6 | kelch like family member 6 | −1.683531539 |
| | member 16 | | | | |
| ARHGAP19 | Rho GTPase activating | 3.167705534 | ELMO2 | engulfment and cell motility | −1.683696454 |
| | protein 19 | | | 2 | |
| TYMS-PS | thymidylate synthase, | 3.166362514 | POLR3D | polymerase (RNA) III | −1.683942043 |
| | pseudogene(Tyms-ps) | | | subunit D | |
| IL3RA | interleukin 3 receptor | 3.145793675 | RALGPS1 | Ral GEF with PH domain | −1.685524532 |
| | subunit alpha | | | and SH3 binding motif 1 | |
| TMEM53 | transmembrane protein 53 | 3.141596278 | ATL2 | atlastin GTPase 2 | −1.685731341 |
| THNSL2 | threonine synthase like 2 | 3.141596278 | RAD52 | RAD52 homolog, DNA | −1.689523672 |
| | | | | repair protein | |
| 2810408M09RIK | Description Not Found | 3.129283017 | GPC1 | glypican 1 | −1.689646894 |
| ADAMDEC1 | ADAM like decysin 1 | 3.121015401 | ARHGAP15 | Rho GTPase activating | −1.690804518 |
| | | | | protein 15 | |
| ASB2 | ankyrin repeat and SOCS | 3.118792343 | GPRC5B | G protein-coupled receptor | −1.693999744 |
| | box containing 2 | | | class C group 5 member B | |
| SLC37A4 | solute carrier family 37 | 3.112700133 | ZBTB1 | zinc finger and BTB | −1.694046727 |
| | member 4 | | | domain containing 1 | |
| NICN1 | nicolin 1 | 3.108478268 | NARFL | nuclear prelamin A | −1.694880193 |
| | | | | recognition factor like | |
| 2310067B10RIK | Description Not Found | 3.087462841 | SLC26A6 | solute carrier family 26 | −1.695252347 |
| | | | | member 6 | |
| PIGL | phosphatidylinositol | 3.077239787 | MAPKBP1 | mitogen-activated protein | −1.695908738 |
| | glycan anchor | | | kinase binding protein 1 | |
| | biosynthesis class L | | | | |
| 1190005I06RIK | Description Not Found | 3.070389328 | RAB6B | RAB6B, member RAS | −1.697541036 |
| | | | | oncogene family | |
| DHFR | dihydrofolate reductase | 3.070389328 | ARL2 | ADP ribosylation factor like | −1.700349879 |
| | | | | GTPase 2 | |
| FABP5 | fatty acid binding protein | 3.06608919 | ZFP646 | zinc finger protein | −1.700439718 |
| | 5 | | | 646(Zfp646) | |
| POMT2 | protein O- | 3.055794286 | SELENBP2 | selenium binding protein | −1.700439718 |
| | mannosyltransferase 2 | | | 2(Selenbp2) | |
| F2RL2 | coagulation factor II | 3.053111336 | ACOT3 | acyl-CoA thioesterase | −1.700439718 |
| | thrombin receptor like 2 | | | 3(Acot3) | |
| GRB7 | growth factor receptor | 3.048852907 | REG3G | regenerating family | −1.700439718 |
| | bound protein 7 | | | member 3 gamma | |
| SNX21 | sorting nexin family | 3.044394119 | GAB1 | GRB2 associated binding | −1.700439718 |
| | member 21 | | | protein 1 | |
| SUFU | SUFU negative regulator | 3.044394119 | LCN10 | lipocalin 10 | −1.700439718 |
| | of hedgehog signaling | | | | |
| RFC3 | replication factor C | 3.029288361 | MTHFD2L | methylenetetrahydrofolate | −1.700439718 |
| | subunit 3 | | | dehydrogenase (NADP+ | |
| | | | | dependent) 2-like | |
| CLDN12 | claudin 12 | 3.017921908 | PTCD3 | pentatricopeptide repeat | −1.700439718 |
| | | | | domain 3 | |
| C1QTNF6 | C1q and tumor necrosis | 3.014450679 | NTHL1 | nth-like DNA glycosylase 1 | −1.700439718 |
| | factor related protein 6 | | | | |
| PLCXD1 | phosphatidylinositol | 2.99095486 | NUDT3 | nudix hydrolase 3 | −1.700439718 |
| | specific phospholipase C | | | | |
| | X domain containing 1 | | | | |
| SULT4A1 | sulfotransferase family | 2.99095486 | CLEC12A | C-type lectin domain family | −1.700439718 |
| | 4A member 1 | | | 12 member A | |
| CTTNBP2NL | CTTNBP2 N-terminal | 2.981852653 | ZBTB3 | zinc finger and BTB | −1.700439718 |
| | like | | | domain containing 3 | |
| SNX5 | sorting nexin 5 | 2.977279924 | AMT | aminomethyltransferase | −1.700439718 |
| HPS5 | HPS5, biogenesis of | 2.972692654 | ZDHHC14 | zinc finger DHHC-type | −1.700439718 |
| | lysosomal organelles | | | containing 14 | |
| | complex 2 subunit 2 | | | | |
| WISP1 | WNT1 inducible | 2.968090752 | NKX2-5 | NK2 homeobox 5 | −1.700491519 |
| | signaling pathway protein | | | | |
| | 1 | | | | |
| PTPN9 | protein tyrosine | 2.963474124 | FOXA3 | forkhead box A3 | −1.702815694 |
| | phosphatase, non-receptor | | | | |
| | type 9 | | | | |
| USP37 | ubiquitin specific | 2.95419631 | WASF1 | WAS protein family | −1.706412734 |
| | peptidase 37 | | | member 1 | |
| SH3BGRL | SH3 domain binding | 2.935459748 | OLFR690 | olfactory receptor | −1.707192688 |
| | glutamate rich protein like | | | 690(Olfr690) | |
| NCALD | neurocalcin delta | 2.935459748 | ENTPD5 | ectonucleoside triphosphate | −1.707764551 |
| | | | | diphosphohydrolase 5 | |
| CDC42EP4 | CDC42 effector protein 4 | 2.916476644 | PCDHGA4 | protocadherin gamma | −1.709042655 |
| | | | | subfamily A, 4 | |
| IGFBP7 | insulin like growth factor | 2.910553168 | TCF12 | transcription factor 12 | −1.710308209 |
| | binding protein 7 | | | | |
| ABHD4 | abhydrolase domain | 2.908868748 | MTRR | 5-methyltetrahydrofolate- | −1.711494907 |
| | containing 4 | | | homocysteine | |
| | | | | methyltransferase reductase | |
| CSF1 | colony stimulating factor | 2.906890596 | CDKN1C | cyclin dependent kinase | −1.711690028 |
| | 1 | | | inhibitor 1C | |
| COX7A1 | cytochrome c oxidase | 2.897240426 | PRICKLE1 | prickle planar cell polarity | −1.713410822 |
| | subunit 7A1 | | | protein 1 | |
| TTYH2 | tweety family member 2 | 2.892391026 | ATXN7L1 | ataxin 7 like 1 | −1.71669984 |
| ACO1 | aconitase 1 | 2.87774425 | SLCO3A1 | solute carrier organic anion | −1.719235762 |
| | | | | transporter family member | |
| | | | | 3A1 | |
| BARD1 | BRCA1 associated RING | 2.867896464 | TMEM110 | transmembrane protein 110 | −1.720046704 |
| | domain 1 | | | | |
| GPN1 | GPN-loop GTPase 1 | 2.867896464 | KLF2 | Kruppel like factor 2 | −1.721374729 |
| PTTG1 | pituitary tumor- | 2.867896464 | FGG | fibrinogen gamma chain | −1.722466024 |
| | transforming 1 | | | | |
| 2810408A11RIK | Description Not Found | 2.857980995 | ASAH2 | N-acylsphingosine | −1.722466024 |
| | | | | amidohydrolase 2 | |
| BBX | BBX, HMG-box | 2.857980995 | LAP3 | leucine aminopeptidase 3 | −1.722466024 |
| | containing | | | | |
| LTBP3 | latent transforming | 2.837943242 | STAB2 | stabilin 2 | −1.722466024 |
| | growth factor beta | | | | |
| | binding protein 3 | | | | |
| ACTG2 | actin, gamma 2, smooth | 2.827819025 | IL22RA1 | interleukin 22 receptor | −1.722466024 |
| | muscle, enteric | | | subunit alpha 1 | |
| ISLR | immunoglobulin | 2.827819025 | SERINC4 | serine incorporator 4 | −1.722466024 |
| | superfamily containing | | | | |
| | leucine rich repeat | | | | |
| NARS2 | asparaginyl-tRNA | 2.823087408 | GPR180 | G protein-coupled receptor | −1.722466024 |
| | synthetase 2, | | | 180 | |
| | mitochondrial (putative) | | | | |
| ICAM4 | intercellular adhesion | 2.81452379 | TIPARP | TCDD inducible | −1.722466024 |
| | molecule 4 (Landsteiner- | | | poly(ADP-ribose) | |
| | Wiener blood group) | | | polymerase | |
| ABCB8 | ATP binding cassette | 2.813358991 | USP11 | ubiquitin specific peptidase | −1.722466024 |
| | subfamily B member 8 | | | 11 | |
| IDI1 | isopentenyl-diphosphate | 2.811782922 | TRIP6 | thyroid hormone receptor | −1.722466024 |
| | delta isomerase 1 | | | interactor 6 | |
| GLS2 | glutaminase 2 | 2.797012978 | KCNH2 | potassium voltage-gated | −1.722466024 |
| | | | | channel subfamily H | |
| | | | | member 2 | |
| HDAC8 | histone deacetylase 8 | 2.797012978 | ESR2 | estrogen receptor 2 | −1.722466024 |
| BRIP1 | BRCA1 interacting | 2.797012978 | FGF13 | fibroblast growth factor 13 | −1.722639247 |
| | protein C-terminal | | | | |
| | helicase 1 | | | | |
| USP6NL | USP6 N-terminal like | 2.794415866 | KBTBD7 | kelch repeat and BTB | −1.724237927 |
| | | | | domain containing 7 | |
| TLCD2 | TLC domain containing 2 | 2.791814071 | UHRF1BP1 | UHRF1 binding protein 1 | −1.725835292 |
| GUCY1A3 | guanylate cyclase 1 | 2.787502763 | BCAM | basal cell adhesion | −1.726509704 |
| | soluble subunit alpha | | | molecule (Lutheran blood | |
| | | | | group) | |
| OCA2 | OCA2 melanosomal | 2.786596362 | ELOVL6 | ELOVL fatty acid elongase | −1.726565554 |
| | transmembrane protein | | | 6 | |
| VAT1 | vesicle amine transport 1 | 2.772502543 | PPM1K | protein phosphatase, | −1.726643643 |
| | | | | Mg2+/Mn2+ dependent 1K | |
| HIST1H2AB | histone cluster 1, H2ab | 2.767914142 | SPATA6 | spermatogenesis associated | −1.727673077 |
| | | | | 6 | |
| PIGC | phosphatidylinositol | 2.760220946 | NAV1 | neuron navigator 1 | −1.727920455 |
| | glycan anchor | | | | |
| | biosynthesis class C | | | | |
| PARG | poly(ADP-ribose) | 2.756558208 | ANK3 | ankyrin 3, node of Ranvier | −1.727920455 |
| | glycohydrolase | | | (ankyrin G) | |
| ESCO2 | establishment of sister | 2.754887502 | KCNAB1 | potassium voltage-gated | −1.727920455 |
| | chromatid cohesion N- | | | channel subfamily A | |
| | acetyltransferase 2 | | | member regulatory beta | |
| | | | | subunit 1 | |
| HIPK2 | homeodomain interacting | 2.754887502 | CYP27A1 | cytochrome P450 family 27 | −1.727920455 |
| | protein kinase 2 | | | subfamily A member 1 | |
| IMPA1 | inositol monophosphatase | 2.752945007 | MAP4K4 | mitogen-activated protein | −1.729756006 |
| | 1 | | | kinase kinase kinase kinase | |
| | | | | 4 | |
| COQ4 | coenzyme Q4 | 2.744161096 | ANKRD7 | ankyrin repeat domain 7 | −1.730646873 |
| ZBTB7A | zinc finger and BTB | 2.744161096 | IFRD1 | interferon related | −1.732447522 |
| | domain containing 7A | | | developmental regulator 1 | |
| GAMT | guanidinoacetate N- | 2.744161096 | ALX3 | ALX homeobox 3 | −1.733354341 |
| | methyltransferase | | | | |
| BIK | BCL2 interacting killer | 2.744161096 | SNURF | SNRPN upstream reading | −1.733354341 |
| | | | | frame | |
| PMS1 | PMS1 homolog 1, | 2.733354341 | AMZ2 | archaelysin family | −1.73350053 |
| | mismatch repair system | | | metallopeptidase 2 | |
| | component | | | | |
| HAVCR2 | hepatitis A virus cellular | 2.729769667 | ROGDI | rogdi homolog | −1.73419198 |
| | receptor 2 | | | | |
| FHL2 | four and a half LIM | 2.727254747 | DAGLA | diacylglycerol lipase alpha | −1.734471203 |
| | domains 2 | | | | |
| CHAF1A | chromatin assembly | 2.725248783 | 4930432K21RIK | Description Not Found | −1.736243886 |
| | factor 1 subunit A | | | | |
| 2810004N23RIK | Description Not Found | 2.722466024 | KRCC1 | lysine rich coiled-coil 1 | −1.73665741 |
| TBC1D14 | TBC1 domain family | 2.722466024 | OLFR1331 | olfactory receptor | −1.736826447 |
| | member 14 | | | 1331(Olfr1331) | |
| EHD2 | EH domain containing 2 | 2.711494907 | SLC25A25 | solute carrier family 25 | −1.73690749 |
| | | | | member 25 | |
| APH1A | aph-1 homolog A, | 2.705977902 | CXCR4 | C-X-C motif chemokine | −1.737779353 |
| | gamma-secretase subunit | | | receptor 4 | |
| TMEM2 | transmembrane protein 2 | 2.703211467 | EPB4.1L3 | Description Not Found | −1.738767837 |
| LCAT | lecithin-cholesterol | 2.700439718 | CEP164 | centrosomal protein 164 | −1.738795736 |
| | acyltransferase | | | | |
| FBXO15 | F-box protein 15 | 2.689299161 | AGER | advanced glycosylation end | −1.73961488 |
| | | | | product-specific receptor | |
| ADAP1 | ArfGAP with dual PH | 2.674391397 | B3GALT5 | beta-1,3- | −1.740215306 |
| | domains 1 | | | galactosyltransferase 5 | |
| PPAPDC1B | Description Not Found | 2.666756592 | OLFR450 | olfactory receptor | −1.74228265 |
| | | | | 450(Olfr450) | |
| CD48 | CD48 molecule | 2.666756592 | ZFP780B | zinc finger protein | −1.744161096 |
| | | | | 780B(Zfp780b) | |
| CAMK4 | calcium/calmodulin | 2.655351829 | OLFR485 | olfactory receptor | −1.744161096 |
| | dependent protein kinase | | | 485(Olfr485) | |
| | IV | | | | |
| SAC3D1 | SAC3 domain containing | 2.64385619 | OLFR47 | olfactory receptor | −1.744161096 |
| | 1 | | | 47(Olfr47) | |
| ECHDC2 | enoyl-CoA hydratase | 2.640725033 | CYP4F18 | cytochrome P450, family 4, | −1.744161096 |
| | domain containing 2 | | | subfamily f, polypeptide | |
| | | | | 18(Cyp4f18) | |
| INCENP | inner centromere protein | 2.638460117 | PLOD2 | procollagen-lysine,2- | −1.744161096 |
| | | | | oxoglutarate 5-dioxygenase | |
| | | | | 2 | |
| INTS9 | integrator complex | 2.634920268 | OSBPL1A | oxysterol binding protein | −1.744161096 |
| | subunit 9 | | | like 1A | |
| KLRA17 | killer cell lectin-like | 2.632268216 | CHRNA5 | cholinergic receptor | −1.744161096 |
| | receptor, subfamily A, | | | nicotinic alpha 5 subunit | |
| | member 17(Klra17) | | | | |
| MAN2B2 | mannosidase alpha class | 2.632268216 | TSSK4 | testis specific serine kinase | −1.744161096 |
| | 2B member 2 | | | 4 | |
| DOLK | dolichol kinase | 2.632268216 | ALKBH8 | alkB homolog 8, tRNA | −1.744161096 |
| | | | | methyltransferase | |
| SAP30BP | SAP30 binding protein | 2.632268216 | GPX2 | glutathione peroxidase 2 | −1.744161096 |
| RTN1 | reticulon 1 | 2.627898616 | ATG4D | autophagy related 4D | −1.744161096 |
| | | | | cysteine peptidase | |
| ADAM15 | ADAM metallopeptidase | 2.626439137 | SCRN3 | secernin 3 | −1.744161096 |
| | domain 15 | | | | |
| STAG3 | stromal antigen 3 | 2.62058641 | NOTCH3 | notch 3 | −1.744161096 |
| NUDT2 | nudix hydrolase 2 | 2.610775705 | OLFR113 | olfactory receptor | −1.744357436 |
| | | | | 113(Olfr113) | |
| GLT8D2 | glycosyltransferase 8 | 2.609988757 | CD28 | CD28 molecule | −1.744605653 |
| | domain containing 2 | | | | |
| CAPSL | calcyphosine like | 2.608809243 | SAG | S-antigen; retina and pineal | −1.745224161 |
| | | | | gland (arrestin) | |
| CALR | calreticulin | 2.608809243 | AGTRAP | angiotensin II receptor | −1.749107415 |
| | | | | associated protein | |
| CRYBG3 | crystallin beta-gamma | 2.605393551 | BLK | BLK proto-oncogene, Src | −1.749534268 |
| | domain containing 3 | | | family tyrosine kinase | |
| DIXDC1 | DIX domain containing 1 | 2.596940379 | MGAT5 | mannosyl (alpha-1,6-)- | −1.749534268 |
| | | | | glycoprotein beta-1,6-N- | |
| | | | | acetyl- | |
| | | | | glucosaminyltransferase | |
| TACSTD2 | tumor-associated calcium | 2.593926161 | RNF2 | ring finger protein 2 | −1.750890228 |
| | signal transducer 2 | | | | |
| TRP53RK | Description Not Found | 2.588066506 | COL14A1 | collagen type XIV alpha 1 | −1.752093722 |
| | | | | chain | |
| PDCD1LG2 | programmed cell death 1 | 2.584962501 | PLEKHG3 | pleckstrin homology and | −1.752109698 |
| | ligand 2 | | | RhoGEF domain containing | |
| | | | | G3 | |
| SEC23IP | SEC23 interacting protein | 2.584962501 | ARHGEF18 | Rho/Rac guanine nucleotide | −1.754100479 |
| | | | | exchange factor 18 | |
| ORM1 | orosomucoid 1 | 2.584962501 | LEF1 | lymphoid enhancer binding | −1.754887502 |
| | | | | factor 1 | |
| ZFP322A | zinc finger protein | 2.575024164 | COMMD9 | COMM domain containing | −1.75490709 |
| | 322A(Zfp322a) | | | 9 | |
| 4931406C07RIK | Description Not Found | 2.560714954 | SLC20A1 | solute carrier family 20 | −1.758637847 |
| | | | | member 1 | |
| ZFP382 | zinc finger protein | 2.560714954 | ACTR5 | ARP5 actin-related protein | −1.759244091 |
| | 382(Zfp382) | | | 5 homolog | |
| CLIP2 | CAP-Gly domain | 2.560714954 | UBQLN3 | ubiquilin 3 | −1.765109548 |
| | containing linker protein | | | | |
| | 2 | | | | |
| TNFAIP8L1 | TNF alpha induced | 2.560714954 | ZFP770 | zinc finger protein | −1.765534746 |
| | protein 8 like 1 | | | 770(Zfp770) | |
| NRCAM | neuronal cell adhesion | 2.560714954 | PCDHB18 | protocadherin beta | −1.765534746 |
| | molecule | | | 18(Pcdhb18) | |
| HPSE | heparanase | 2.560714954 | OLFR700 | olfactory receptor | −1.765534746 |
| | | | | 700(Olfr700) | |
| RTKN | rhotekin | 2.558985655 | FOXP4 | forkhead box P4 | −1.765534746 |
| DLGAP5 | DLG associated protein 5 | 2.550125328 | CDC34 | cell division cycle 34 | −1.765534746 |
| ENPP2 | ectonucleotide | 2.548436625 | HIST1H1E | histone cluster 1, H1e | −1.765534746 |
| | pyrophosphatase/ | | | | |
| | phosphodiesterase 2 | | | | |
| GCNT1 | glucosaminyl (N-acetyl) | 2.548436625 | G6PC2 | glucose-6-phosphatase | −1.765534746 |
| | transferase 1, core 2 | | | catalytic subunit 2 | |
| SASS6 | SAS-6 centriolar | 2.548436625 | FUT1 | fucosyltransferase 1 | −1.765534746 |
| | assembly protein | | | (H blood group) | |
| AMIGO3 | adhesion molecule with | 2.548436625 | ZFP69 | ZFP69 zinc finger protein | −1.765534746 |
| | Ig-like domain 3 | | | | |
| APH1B | aph-1 homolog B, | 2.548436625 | WBSCR27 | Williams Beuren syndrome | −1.765534746 |
| | gamma-secretase subunit | | | chromosome region 27 | |
| ABCC5 | ATP binding cassette | 2.547846505 | METTL8 | methyltransferase like 8 | −1.766880868 |
| | subfamily C member 5 | | | | |
| YIPF6 | Yip1 domain family | 2.543805176 | TMEM170 | transmembrane protein | −1.767462508 |
| | member 6 | | | 170(Tmem170) | |
| FFAR1 | free fatty acid receptor 1 | 2.5360529 | TRP53INP1 | transformation related | −1.767518474 |
| | | | | protein 53 inducible nuclear | |
| | | | | protein 1(Trp53inp1) | |
| TSSK6 | testis specific serine | 2.5360529 | H2-Q5 | histocompatibility 2, Q | −1.769676967 |
| | kinase 6 | | | region locus 5(H2-Q5) | |
| ETV6 | ETS variant 6 | 2.535385323 | ADCK1 | aarF domain containing | −1.770033995 |
| | | | | kinase 1 | |
| PTGDS | prostaglandin D2 | 2.529838423 | IMPAD1 | inositol monophosphatase | −1.771434505 |
| | synthase | | | domain containing 1 | |
| SH3D19 | SH3 domain containing | 2.523561956 | E4F1 | E4F transcription factor 1 | −1.772427885 |
| | 19 | | | | |
| KIF5C | kinesin family member | 2.518298014 | ZFYVE20 | Description Not Found | −1.772942676 |
| | 5C | | | | |
| PTGER2 | prostaglandin E receptor 2 | 2.517275693 | PNPLA6 | patatin like phospholipase | −1.775074114 |
| | | | | domain containing 6 | |
| INSR | insulin receptor | 2.510961919 | TRIB3 | tribbles pseudokinase 3 | −1.775215233 |
| MAPK6 | mitogen-activated protein | 2.504620392 | GM614 | predicted gene 614(Gm614) | −1.776103988 |
| | kinase 6 | | | | |
| OXSR1 | oxidative stress | 2.502211192 | D5ERTD579E | DNA segment, Chr 5, | −1.776306798 |
| | responsive 1 | | | ERATO Doi 579, | |
| | | | | expressed(D5Ertd579e) | |
| EZH2 | enhancer of zeste 2 | 2.501439145 | SCAND1 | SCAN domain containing 1 | −1.77785827 |
| | polycomb repressive | | | | |
| | complex 2 subunit | | | | |
| BNIP1 | BCL2 interacting protein | 2.498250868 | ASB13 | ankyrin repeat and SOCS | −1.782205107 |
| | 1 | | | box containing 13 | |
| LPCAT4 | lysophosphatidylcholine | 2.495285165 | ARHGEF4 | Rho guanine nucleotide | −1.784072601 |
| | acyltransferase 4 | | | exchange factor 4 | |
| PPAP2C | Description Not Found | 2.485426827 | H1FNT | H1 histone family member | −1.78485543 |
| | | | | N, testis specific | |
| IFNA12 | interferon alpha | 2.485426827 | BLOC1S1 | biogenesis of lysosomal | −1.784911393 |
| | 12(Ifna12) | | | organelles complex 1 | |
| | | | | subunit 1 | |
| DCLK1 | doublecortin like kinase 1 | 2.485426827 | ZFYVE27 | zinc finger FYVE-type | −1.7851013 |
| | | | | containing 27 | |
| MX1 | MX dynamin like GTPase | 2.485426827 | RHOX4B | reproductive homeobox | −1.786596362 |
| | 1 | | | 4B(Rhox4b) | |
| SMTN | smoothelin | 2.485426827 | OLFR1134 | olfactory receptor | −1.786596362 |
| | | | | 1134(Olfr1134) | |
| PLA2G15 | phospholipase A2 group | 2.48194563 | CAR11 | carbonic anhydrase | −1.786596362 |
| | XV | | | 11(Car11) | |
| OLFR192 | olfactory receptor | 2.472487771 | LRRIQ4 | leucine rich repeats and IQ | −1.786596362 |
| | 192(Olfr192) | | | motif containing 4 | |
| ITGB5 | integrin subunit beta 5 | 2.472487771 | CASP12 | caspase 12 | −1.786596362 |
| | | | | (gene/pseudogene) | |
| RAPSN | receptor associated | 2.465974465 | ODF3L1 | outer dense fiber of sperm | −1.786596362 |
| | protein of the synapse | | | tails 3 like 1 | |
| SNX3 | sorting nexin 3 | 2.459431619 | CCDC3 | coiled-coil domain | −1.786596362 |
| | | | | containing 3 | |
| FERMT2 | fermitin family member 2 | 2.459431619 | SSPN | sarcospan | −1.786596362 |
| CCR5 | C-C motif chemokine | 2.444410478 | KLK1 | kallikrein 1 | −1.786596362 |
| | receptor 5 | | | | |
| | (gene/pseudogene) | | | | |
| UPK1A | uroplakin 1A | 2.439623138 | SENP7 | SUMO1/sentrin specific | −1.786897131 |
| | | | | peptidase 7 | |
| BCL2L2 | BCL2 like 2 | 2.43629512 | CAML | calcium modulating | −1.787735284 |
| | | | | ligand(Caml) | |
| 2610002M06RIK | Description Not Found | 2.432959407 | YEATS2 | YEATS domain containing | −1.788627083 |
| | | | | 2 | |
| CENPN | centromere protein N | 2.432959407 | SERPINF2 | serpin family F member 2 | −1.791814071 |
| HBEGF | heparin binding EGF like | 2.43096254 | KCNMB1 | potassium calcium- | −1.792597191 |
| | growth factor | | | activated channel subfamily | |
| | | | | M regulatory beta subunit 1 | |
| TYMS | thymidylate synthetase | 2.427103287 | FCHO2 | FCH domain only 2 | −1.792666489 |
| MGA | MGA, MAX dimerization | 2.426939834 | BBS9 | Bardet-Biedl syndrome 9 | −1.792734984 |
| | protein | | | | |
| RAI14 | retinoic acid induced 14 | 2.426264755 | OLFR323 | olfactory receptor | −1.794609131 |
| | | | | 323(Olfr323) | |
| CFI | complement factor I | 2.419538892 | CD247 | CD247 molecule | −1.796081585 |
| PLK4 | polo like kinase 4 | 2.419538892 | HIST2H2AA1 | histone cluster 2, | −1.796847743 |
| | | | | H2aa1(Hist2h2aa1) | |
| SLC6A9 | solute carrier family 6 | 2.419538892 | PDK1 | pyruvate dehydrogenase | −1.800563818 |
| | member 9 | | | kinase 1 | |
| TMED2 | transmembrane p24 | 2.419538892 | NRARP | NOTCH-regulated ankyrin | −1.803049246 |
| | trafficking protein 2 | | | repeat protein | |
| TMEM120B | transmembrane protein | 2.41857423 | BTBD11 | BTB domain containing 11 | −1.804793263 |
| | 120B | | | | |
| TRIM36 | tripartite motif containing | 2.417852515 | CSF2RA | colony stimulating factor 2 | −1.805089518 |
| | 36 | | | receptor alpha subunit | |
| CCDC93 | coiled-coil domain | 2.416164165 | DEXI | Dexi homolog | −1.806998156 |
| | containing 93 | | | | |
| SLC25A35 | solute carrier family 25 | 2.409367225 | OLFR1276 | olfactory receptor | −1.807354922 |
| | member 35 | | | 1276(Olfr1276) | |
| BNC1 | basonuclin 1 | 2.40599236 | TCSTV3 | 2-cell-stage, variable group, | −1.807354922 |
| | | | | member 3(Tcstv3) | |
| FOXL2 | forkhead box L2 | 2.40599236 | SPRR2D | small proline rich protein | −1.807354922 |
| | | | | 2D | |
| TFPI2 | tissue factor pathway | 2.40599236 | SEMA4G | semaphorin 4G | −1.807354922 |
| | inhibitor 2 | | | | |
| NET1 | neuroepithelial cell | 2.40599236 | KCNK9 | potassium two pore domain | −1.807354922 |
| | transforming 1 | | | channel subfamily K | |
| | | | | member 9 | |
| SLCO2A1 | solute carrier organic | 2.40599236 | SNAPC3 | small nuclear RNA | −1.807385513 |
| | anion transporter family | | | activating complex | |
| | member 2A1 | | | polypeptide 3 | |
| A730008H23RIK | Description Not Found | 2.399275037 | AXIN2 | axin 2 | −1.808429403 |
| CDKN2B | cyclin dependent kinase | 2.397264578 | PCNXL3 | Description Not Found | −1.808995133 |
| | inhibitor 2B | | | | |
| ZFP532 | zinc finger protein | 2.393138801 | KLHL7 | kelch like family member 7 | −1.809016035 |
| | 532(Zfp532) | | | | |
| GTSE1 | G2 and S-phase expressed | 2.392428431 | ZFP281 | zinc finger protein | −1.811556991 |
| | 1 | | | 281(Zfp281) | |
| CCDC14 | coiled-coil domain | 2.392317423 | CHRNB2 | cholinergic receptor | −1.812498225 |
| | containing 14 | | | nicotinic beta 2 subunit | |
| ADAT1 | adenosine deaminase, | 2.392317423 | TBC1D15 | TBC1 domain family | −1.812909044 |
| | tRNA specific 1 | | | member 15 | |
| DGKH | diacylglycerol kinase eta | 2.392317423 | GALNT9 | polypeptide N- | −1.813407449 |
| | | | | acetylgalactosaminyl- | |
| | | | | transferase 9 | |
| ZRSR1 | zinc finger CCCH-type, | 2.392317423 | DYNC1I1 | dynein cytoplasmic 1 | −1.813434179 |
| | RNA binding motif and | | | intermediate chain 1 | |
| | serine/arginine rich 1 | | | | |
| NFE2 | nuclear factor, erythroid 2 | 2.391529377 | MYH8 | myosin heavy chain 8 | −1.81403224 |
| CD63 | CD63 molecule | 2.387853137 | CEP57 | centrosomal protein 57 | −1.815684972 |
| MIB1 | mindbomb E3 ubiquitin | 2.38645559 | LTK | leukocyte receptor tyrosine | −1.817623258 |
| | protein ligase 1 | | | kinase | |
| TSN | translin | 2.382349023 | COMMD2 | COMM domain containing | −1.817623258 |
| | | | | 2 | |
| 2510003E04RIK | Description Not Found | 2.378511623 | MEF2C | myocyte enhancer factor 2C | −1.817623258 |
| BC043934 | cDNA sequence | 2.378511623 | LONRF2 | LON peptidase N-terminal | −1.817941412 |
| | BC043934(BC043934) | | | domain and ring finger 2 | |
| AHCYL1 | adenosylhomocysteinase | 2.366734247 | PDCD6IP | programmed cell death 6 | −1.820575529 |
| | like 1 | | | interacting protein | |
| OLFR731 | olfactory receptor | 2.364572432 | DHX16 | DEAH-box helicase 16 | −1.820661084 |
| | 731(Olfr731) | | | | |
| CDKN2A | cyclin dependent kinase | 2.364572432 | ZFYVE19 | zinc finger FYVE-type | −1.825281028 |
| | inhibitor 2A | | | containing 19 | |
| SLC29A4 | solute carrier family 29 | 2.364572432 | H2-T10 | histocompatibility 2, T | −1.826218639 |
| | member 4 | | | region locus 10(H2-T10) | |
| SLC4A10 | solute carrier family 4 | 2.364572432 | ARID1A | AT-rich interaction domain | −1.827043205 |
| | member 10 | | | 1A | |
| CYCS | cytochrome c, somatic | 2.351872866 | NOD1 | nucleotide binding | −1.827185706 |
| | | | | oligomerization domain | |
| | | | | containing 1 | |
| COL5A1 | collagen type V alpha 1 | 2.350497247 | 2610318N02RIK | Description Not Found | −1.827819025 |
| UTRN | utrophin | 2.350497247 | BC048644 | cDNA sequence | −1.827819025 |
| | | | | BC048644(BC048644) | |
| AURKA | aurora kinase A | 2.349678136 | CDC42EP2 | CDC42 effector protein 2 | −1.827819025 |
| KREMEN2 | kringle containing | 2.349431709 | CCL25 | C-C motif chemokine | −1.827819025 |
| | transmembrane protein 2 | | | ligand 25 | |
| FGL2 | fibrinogen like 2 | 2.346409407 | TBX6 | T-box 6 | −1.827819025 |
| NCAM1 | neural cell adhesion | 2.343407822 | PLEKHG4 | pleckstrin homology and | −1.827819025 |
| | molecule 1 | | | RhoGEF domain containing | |
| | | | | G4 | |
| ALG8 | ALG8, alpha-1,3- | 2.343407822 | RAD18 | RAD18, E3 ubiquitin | −1.830642494 |
| | glucosyltransferase | | | protein ligase | |
| OLFR703 | olfactory receptor | 2.336283388 | SLC12A9 | solute carrier family 12 | −1.830807586 |
| | 703(Olfr703) | | | member 9 | |
| SLC39A10 | solute carrier family 39 | 2.336283388 | NR1D2 | nuclear receptor subfamily | −1.837943242 |
| | member 10 | | | 1 group D member 2 | |
| HIST1H2AH | histone cluster 1, H2ah | 2.322141712 | NLK | nemo like kinase | −1.840170811 |
| TSGA8 | testis specific gene | 2.321928095 | TTC37 | tetratricopeptide repeat | −1.840462743 |
| | A8(Tsga8) | | | domain 37 | |
| ELOVL2 | ELOVL fatty acid | 2.321928095 | DLG3 | discs large MAGUK | −1.841507525 |
| | elongase 2 | | | scaffold protein 3 | |
| MLF1 | myeloid leukemia factor 1 | 2.321928095 | PCF11 | PCF11 cleavage and | −1.843349827 |
| | | | | polyadenylation factor | |
| | | | | subunit | |
| FZD6 | frizzled class receptor 6 | 2.321928095 | HIST1H4D | histone cluster 1, H4d | −1.846386944 |
| PLD1 | phospholipase D1 | 2.321928095 | PEX26 | peroxisomal biogenesis | −1.847440096 |
| | | | | factor 26 | |
| IFRD2 | interferon-related | 2.321928095 | CYP2B10 | cytochrome P450, family 2, | −1.847996907 |
| | developmental regulator 2 | | | subfamily b, polypeptide | |
| | | | | 10(Cyp2b10) | |
| OLA1 | Obg-like ATPase 1 | 2.321928095 | GDF3 | growth differentiation | −1.847996907 |
| | | | | factor 3 | |
| ASPA | aspartoacylase | 2.321928095 | GPR33 | G protein-coupled receptor | −1.847996907 |
| | | | | 33 (gene/pseudogene) | |
| TGFB3 | transforming growth | 2.321928095 | TDG | thymine DNA glycosylase | −1.847996907 |
| | factor beta 3 | | | | |
| PKIG | protein kinase (cAMP- | 2.314696526 | HIPK3 | homeodomain interacting | −1.847996907 |
| | dependent, catalytic) | | | protein kinase 3 | |
| | inhibitor gamma | | | | |
| TNFRSF4 | tumor necrosis factor | 2.308832886 | PAPOLA | poly(A) polymerase alpha | −1.847996907 |
| | receptor superfamily | | | | |
| | member 4 | | | | |
| IQCB1 | IQ motif containing B1 | 2.307984443 | MAPK4 | mitogen-activated protein | −1.847996907 |
| | | | | kinase 4 | |
| SLC16A11 | solute carrier family 16 | 2.307662797 | FRAT2 | frequently rearranged in | −1.84969115 |
| | member 11 | | | advanced T-cell lymphomas | |
| | | | | 2 | |
| 1190002N15RIK | Description Not Found | 2.307428525 | HEXIM1 | hexamethylene | −1.851035845 |
| | | | | bisacetamide inducible 1 | |
| LCE1L | late cornified envelope | 2.307428525 | TATDN2 | TatD DNase domain | −1.851433223 |
| | 1L(Lce1l) | | | containing 2 | |
| RGS13 | regulator of G-protein | 2.307428525 | KLRB1C | killer cell lectin-like | −1.854253843 |
| | signaling 13 | | | receptor subfamily B | |
| | | | | member 1C(Klrb1c) | |
| FBXW8 | F-box and WD repeat | 2.299987517 | SLC16A9 | solute carrier family 16 | −1.855083462 |
| | domain containing 8 | | | member 9 | |
| SNCA | synuclein alpha | 2.296457407 | ACBD4 | acyl-CoA binding domain | −1.855739032 |
| | | | | containing 4 | |
| OSGIN1 | oxidative stress induced | 2.294491702 | REXO1 | RNA exonuclease 1 | −1.857980995 |
| | growth inhibitor 1 | | | homolog | |
| BC004004 | cDNA sequence | 2.292781749 | OLFR1442 | olfactory receptor | −1.859286959 |
| | BC004004(BC004004) | | | 1442(Olfr1442) | |
| WNT10A | Wnt family member 10A | 2.292781749 | PHOSPHO1 | phosphoethanolamine/ | −1.859747926 |
| | | | | phosphocholine | |
| | | | | phosphatase | |
| THG1L | tRNA-histidine | 2.292781749 | ITPKA | inositol-trisphosphate 3- | −1.859881803 |
| | guanylyltransferase 1 like | | | kinase A | |
| MLH1 | mutL homolog 1 | 2.292781749 | ZFHX2 | zinc finger homeobox 2 | −1.860513882 |
| RRM2 | ribonucleotide reductase | 2.289435485 | TOR1A | torsin family 1 member A | −1.860949348 |
| | regulatory subunit M2 | | | | |
| SHISA4 | shisa family member 4 | 2.277984747 | CDKAL1 | CDK5 regulatory subunit | −1.862794137 |
| | | | | associated protein 1 like 1 | |
| DDAH2 | dimethylarginine | 2.277984747 | SMAD1 | SMAD family member 1 | −1.863462947 |
| | dimethylaminohydrolase | | | | |
| | 2 | | | | |
| APBA1 | amyloid beta precursor | 2.269085766 | ZC3H13 | zinc finger CCCH-type | −1.863535399 |
| | protein binding family A | | | containing 13 | |
| | member 1 | | | | |
| MMAB | methylmalonic aciduria | 2.264911693 | ZSCAN20 | zinc finger and SCAN | −1.863962106 |
| | (cobalamin deficiency) | | | domain containing 20 | |
| | cblB type | | | | |
| DIAP1 | Description Not Found | 2.263034406 | EPB4.1L4A | Description Not Found | −1.867896464 |
| CAR14 | carbonic anhydrase | 2.263034406 | ZFP280C | zinc finger protein | −1.867896464 |
| | 14(Car14) | | | 280C(Zfp280c) | |
| C2 | complement component 2 | 2.263034406 | GM1322 | predicted gene | −1.867896464 |
| | | | | 1322(Gm1322) | |
| MAG | myelin associated | 2.263034406 | OLFR472 | olfactory receptor | −1.867896464 |
| | glycoprotein | | | 472(Olfr472) | |
| KCNIP3 | potassium voltage-gated | 2.263034406 | OLFR171 | olfactory receptor | −1.867896464 |
| | channel interacting | | | 171(Olfr171) | |
| | protein 3 | | | | |
| CFD | complement factor D | 2.263034406 | OLFR1249 | olfactory receptor | −1.867896464 |
| | | | | 1249(Olfr1249) | |
| CCNE1 | cyclin E1 | 2.262723645 | PRH1 | proline rich protein HaeIII | −1.867896464 |
| | | | | subfamily 1 | |
| RYR1 | ryanodine receptor 1 | 2.261305322 | ARSI | arylsulfatase family | −1.867896464 |
| | | | | member I | |
| PROC | protein C, inactivator of | 2.255500733 | KRT7 | keratin 7 | −1.867896464 |
| | coagulation factors Va | | | | |
| | and VIIIa | | | | |
| ZFP27 | zinc finger protein | 2.247927513 | PCGF3 | polycomb group ring finger | −1.867896464 |
| | 27(Zfp27) | | | 3 | |
| TBX1 | T-box 1 | 2.247927513 | PCTP | phosphatidylcholine | −1.867896464 |
| | | | | transfer protein | |
| DHRS13 | dehydrogenase/reductase | 2.247927513 | CALD1 | caldesmon 1 | −1.867896464 |
| | 13 | | | | |
| HSPG2 | heparan sulfate | 2.247927513 | TREML2 | triggering receptor | −1.867896464 |
| | proteoglycan 2 | | | expressed on myeloid cells | |
| | | | | like 2 | |
| FRMD8 | FERM domain containing | 2.24777312 | RTN4RL1 | reticulon 4 receptor like 1 | −1.867896464 |
| | 8 | | | | |
| MIOX | myo-inositol oxygenase | 2.240579987 | PARVA | parvin alpha | −1.868479018 |
| LYRM1 | LYR motif containing 1 | 2.232660757 | NPCD | neuronal pentraxin chromo | −1.871902039 |
| | | | | domain(Npcd) | |
| STAP1 | signal transducing adaptor | 2.232660757 | RFXANK | regulatory factor X | −1.87206109 |
| | family member 1 | | | associated ankyrin | |
| | | | | containing protein | |
| NAT2 | N-acetyltransferase 2 | 2.232660757 | MAP3K14 | mitogen-activated protein | −1.872291304 |
| | | | | kinase kinase kinase 14 | |
| SRGAP3 | SLIT-ROBO Rho GTPase | 2.232660757 | KLHL9 | kelch like family member 9 | −1.874528943 |
| | activating protein 3 | | | | |
| NXT2 | nuclear transport factor 2 | 2.232660757 | SESN1 | sestrin 1 | −1.875260951 |
| | like export factor 2 | | | | |
| RCOR1 | REST corepressor 1 | 2.232660757 | ADAMTS7 | ADAM metallopeptidase | −1.879404807 |
| | | | | with thrombospondin type 1 | |
| | | | | motif 7 | |
| SRR | serine racemase | 2.230836503 | SNAPC1 | small nuclear RNA | −1.88488993 |
| | | | | activating complex | |
| | | | | polypeptide 1 | |
| IKBKAP | inhibitor of kappa light | 2.226177109 | ADAR | adenosine deaminase, RNA | −1.885299379 |
| | polypeptide gene | | | specific | |
| | enhancer in B-cells, | | | | |
| | kinase complex- | | | | |
| | associated protein | | | | |
| AI597479 | expressed sequence | 2.225819675 | LCE1C | late cornified envelope 1C | −1.885626461 |
| | AI597479(AI597479) | | | | |
| POP1 | POP1 homolog, | 2.224966365 | FBXO21 | F-box protein 21 | −1.886155099 |
| | ribonuclease P/MRP | | | | |
| | subunit | | | | |
| SLC35E4 | solute carrier family 35 | 2.217230716 | 2610524H06RIK | Description Not Found | −1.887525271 |
| | member E4 | | | | |
| XAB2 | XPA binding protein 2 | 2.217230716 | 1700016K19RIK | Description Not Found | −1.887525271 |
| MREG | melanoregulin | 2.2129258 | ZFP715 | zinc finger protein | −1.887525271 |
| | | | | 715(Zfp715) | |
| FKBP11 | FK506 binding protein 11 | 2.210721954 | OLFR446 | olfactory receptor | −1.887525271 |
| | | | | 446(Olfr446) | |
| IGF2BP2 | insulin like growth factor | 2.207789851 | PTK7 | protein tyrosine kinase 7 | −1.887525271 |
| | 2 mRNA binding protein | | | (inactive) | |
| | 2 | | | | |
| NUP133 | nucleoporin 133 | 2.207447199 | TMEM117 | transmembrane protein 117 | −1.887525271 |
| OLFR1183 | olfactory receptor | 2.201633861 | ITIH2 | inter-alpha-trypsin inhibitor | −1.887525271 |
| | 1183(Olfr1183) | | | heavy chain 2 | |
| IL1F6 | interleukin 1 family, | 2.201633861 | TAGLN3 | transgelin 3 | −1.887525271 |
| | member 6(II1f6) | | | | |
| OTX1 | orthodenticle homeobox 1 | 2.201633861 | IFI203 | interferon activated gene | −1.887644112 |
| | | | | 203(Ifi203) | |
| MSH3 | mutS homolog 3 | 2.201633861 | ATP1B1 | ATPase Na+/K+ | −1.887664186 |
| | | | | transporting subunit beta 1 | |
| SCN4B | sodium voltage-gated | 2.201633861 | BLCAP | bladder cancer associated | −1.888596201 |
| | channel beta subunit 4 | | | protein | |
| CROCC | ciliary rootlet coiled-coil, | 2.201633861 | IGF1R | insulin like growth factor 1 | −1.89024137 |
| | rootletin | | | receptor | |
| NSUN2 | NOP2/Sun RNA | 2.194349986 | HMG20A | high mobility group 20A | −1.890579593 |
| | methyltransferase family | | | | |
| | member 2 | | | | |
| GAS2L1 | growth arrest specific 2 | 2.193771743 | WDR24 | WD repeat domain 24 | −1.891527175 |
| | like 1 | | | | |
| 3110007F17RIK | Description Not Found | 2.190740399 | CDX4 | caudal type homeobox 4 | −1.892655439 |
| DEFB15 | defensin beta 15(Defb15) | 2.185866545 | CLDN18 | claudin 18 | −1.893449375 |
| C1QTNF2 | C1q and tumor necrosis | 2.185866545 | IL4RA | interleukin 4 receptor, | −1.895369594 |
| | factor related protein 2 | | | alpha(Il4ra) | |
| RAP1GAP | RAP1 GTPase activating | 2.185866545 | RETNLA | resistin like alpha(Retnla) | −1.895739477 |
| | protein | | | | |
| SNTB1 | syntrophin beta 1 | 2.185866545 | AA388235 | expressed sequence | −1.895739477 |
| | | | | AA388235(AA388235) | |
| FAH | fumarylacetoacetate | 2.182925576 | ZC3H6 | zinc finger CCCH-type | −1.896127489 |
| | hydrolase | | | containing 6 | |
| AVPI1 | arginine vasopressin | 2.174393775 | D930015E06RIK | RIKEN cDNA | −1.899656973 |
| | induced 1 | | | D930015E06 | |
| | | | | gene(D930015E06Rik) | |
| RPA2 | replication protein A2 | 2.172751912 | NPFFR2 | neuropeptide FF receptor 2 | −1.902073579 |
| BRCA2 | BRCA2, DNA repair | 2.168732488 | IRAK1 | interleukin 1 receptor | −1.90243374 |
| | associated | | | associated kinase 1 | |
| RBM47 | RNA binding motif | 2.165911939 | CWF19L2 | CWF19-like 2, cell cycle | −1.903704505 |
| | protein 47 | | | control (S. pombe) | |
| MSL3L2 | male-specific lethal 3-like | 2.159061455 | STK40 | serine/threonine kinase 40 | −1.903964448 |
| | 2 (Drosophila)(Ms1312) | | | | |
| TNFRSF9 | tumor necrosis factor | 2.156071704 | MARS2 | methionyl-tRNA synthetase | −1.904571951 |
| | receptor superfamily | | | 2, mitochondrial | |
| | member 9 | | | | |
| TRF | transferrin(Trf) | 2.154588207 | RAB5A | RAB5A, member RAS | −1.906350687 |
| | | | | oncogene family | |
| ZDHHC15 | zinc finger DHHC-type | 2.154372546 | OLFR1037 | olfactory receptor | −1.906890596 |
| | containing 15 | | | 1037(Olfr1037) | |
| IGJ | Description Not Found | 2.153805336 | ARHGAP22 | Rho GTPase activating | −1.906890596 |
| | | | | protein 22 | |
| FBXO27 | F-box protein 27 | 2.153805336 | DENND1B | DENN domain containing | −1.906890596 |
| | | | | 1B | |
| ZDHHC24 | zinc finger DHHC-type | 2.153805336 | EAPP | E2F associated | −1.906890596 |
| | containing 24 | | | phosphoprotein | |
| SPCS2 | signal peptidase complex | 2.153805336 | ANKRD13D | ankyrin repeat domain 13D | −1.906890596 |
| | subunit 2 | | | | |
| UCN3 | urocortin 3 | 2.153805336 | EFCAB2 | EF-hand calcium binding | −1.906890596 |
| | | | | domain 2 | |
| SLC35A1 | solute carrier family 35 | 2.153805336 | HOXC9 | homeobox C9 | −1.906890596 |
| | member A1 | | | | |
| PODXL | podocalyxin like | 2.153805336 | SENP6 | SUMO1/sentrin specific | −1.907956932 |
| | | | | peptidase 6 | |
| FAM154B | Description Not Found | 2.153792145 | SIDT1 | SID1 transmembrane | −1.908286674 |
| | | | | family member 1 | |
| NRP1 | neuropilin 1 | 2.147470553 | 2310057J18RIK | Description Not Found | −1.916476644 |
| ERGIC1 | endoplasmic reticulum- | 2.147104727 | SPRYD4 | SPRY domain containing 4 | −1.916476644 |
| | golgi intermediate | | | | |
| | compartment 1 | | | | |
| RNF26 | ring finger protein 26 | 2.146810011 | LY6D | lymphocyte antigen 6 | −1.916476644 |
| | | | | complex, locus D | |
| LCN3 | lipocalin 3(Lcn3) | 2.137503524 | PPARGC1B | PPARG coactivator 1 beta | −1.917291956 |
| FMO1 | flavin containing | 2.137503524 | SH3TC1 | SH3 domain and | −1.917906346 |
| | monooxygenase 1 | | | tetratricopeptide repeats 1 | |
| RAB20 | RAB20, member RAS | 2.137503524 | FOXO1 | forkhead box O1 | −1.920209106 |
| | oncogene family | | | | |
| KATNAL1 | katanin catalytic subunit | 2.137503524 | DHX40 | DEAH-box helicase 40 | −1.920623917 |
| | A1 like 1 | | | | |
| GPR107 | G protein-coupled | 2.136424717 | RECQL5 | RecQ like helicase 5 | −1.920664575 |
| | receptor 107 | | | | |
| MELK | maternal embryonic | 2.133399125 | RBM15 | RNA binding motif protein | −1.922616041 |
| | leucine zipper kinase | | | 15 | |
| KCTD9 | potassium channel | 2.13207329 | EGLN2 | egl-9 family hypoxia | −1.924079933 |
| | tetramerization domain | | | inducible factor 2 | |
| | containing 9 | | | | |
| PBK | PDZ binding kinase | 2.130417144 | GPR112 | Description Not Found | −1.925999419 |
| ENPP5 | ectonucleotide | 2.124112676 | OLFR829 | olfactory receptor | −1.925999419 |
| | pyrophosphatase/ | | | 829(Olfr829) | |
| | phosphodiesterase 5 | | | | |
| | (putative) | | | | |
| ZDHHC16 | zinc finger DHHC-type | 2.12361008 | OLFR684 | olfactory receptor | −1.925999419 |
| | containing 16 | | | 684(Olfr684) | |
| OLFR1346 | olfactory receptor | 2.121015401 | RETN | resistin | −1.925999419 |
| | 1346(Olfr1346) | | | | |
| MILL1 | MHC I like leukocyte | 2.121015401 | ST6GALNAC2 | ST6 N- | −1.925999419 |
| | 1(Mill1) | | | acetylgalactosaminide | |
| | | | | alpha-2,6-sialyltransferase 2 | |
| RHCG | Rh family C glycoprotein | 2.121015401 | FES | FES proto-oncogene, | −1.925999419 |
| | | | | tyrosine kinase | |
| CLDN1 | claudin 1 | 2.121015401 | KIF13A | kinesin family member 13A | −1.925999419 |
| LHX3 | LIM homeobox 3 | 2.121015401 | TRPT1 | tRNA phosphotransferase 1 | −1.926457816 |
| TUBB2A | tubulin beta 2A class IIa | 2.121015401 | PLCB2 | phospholipase C beta 2 | −1.927343833 |
| GSG2 | germ cell associated 2, | 2.119412265 | NADSYN1 | NAD synthetase 1 | −1.929674394 |
| | haspin | | | | |
| HYAL2 | hyaluronoglucosaminidase | 2.107345942 | 4833420G17RIK | Description Not Found | −1.93060469 |
| | 2 | | | | |
| 1700003F12RIK | Description Not Found | 2.10433666 | P2RY10 | purinergic receptor P2Y10 | −1.930737338 |
| RUSC2 | RUN and SH3 domain | 2.10433666 | PPAPDC3 | Description Not Found | −1.935459748 |
| | containing 2 | | | | |
| LRRIQ3 | leucine rich repeats and | 2.10433666 | DIP2B | disco interacting protein 2 | −1.935459748 |
| | IQ motif containing 3 | | | homolog B | |
| CHSY1 | chondroitin sulfate | 2.10433666 | RHAG | Rh-associated glycoprotein | −1.935459748 |
| | synthase 1 | | | | |
| DUSP23 | dual specificity | 2.10433666 | EMID1 | EMI domain containing 1 | −1.935459748 |
| | phosphatase 23 | | | | |
| RRAGB | Ras related GTP binding | 2.10433666 | RNF4 | ring finger protein 4 | −1.938834579 |
| | B | | | | |
| KCNAB3 | potassium voltage-gated | 2.10433666 | UBL5 | ubiquitin like 5 | −1.938952478 |
| | channel subfamily A | | | | |
| | regulatory beta subunit 3 | | | | |
| GRPEL2 | GrpE like 2, | 2.103129681 | PROSC | proline synthetase | −1.94016675 |
| | mitochondrial | | | cotranscribed homolog | |
| | | | | (bacterial) | |
| TRAF2 | TNF receptor associated | 2.102029095 | FZD5 | frizzled class receptor 5 | −1.942503137 |
| | factor 2 | | | | |
| COQ7 | coenzyme Q7, | 2.100205246 | UBE2D1 | ubiquitin conjugating | −1.942775467 |
| | hydroxylase | | | enzyme E2 D1 | |
| TMEM126B | transmembrane protein | 2.099187297 | KLRA7 | killer cell lectin-like | −1.943510757 |
| | 126B | | | receptor, subfamily A, | |
| | | | | member 7(Klra7) | |
| SGPL1 | sphingosine-1-phosphate | 2.097112667 | TMEM63C | transmembrane protein 63C | −1.94425562 |
| | lyase 1 | | | | |
| CAPN2 | calpain 2 | 2.096447979 | 2810006K23RIK | Description Not Found | −1.944858446 |
| CHEK2 | checkpoint kinase 2 | 2.088457439 | OLFR672 | olfactory receptor | −1.944858446 |
| | | | | 672(Olfr672) | |
| GLRP1 | glutamine repeat protein | 2.087462841 | OLFR1347 | olfactory receptor | −1.944858446 |
| | 1(Glrp1) | | | 1347(Olfr1347) | |
| RTN4R | reticulon 4 receptor | 2.087462841 | MTTP | microsomal triglyceride | −1.944858446 |
| | | | | transfer protein | |
| TRIM37 | tripartite motif containing | 2.087462841 | MSX1 | msh homeobox 1 | −1.944858446 |
| | 37 | | | | |
| NUCB2 | nucleobindin 2 | 2.087462841 | BSND | barttin CLCNK type | −1.944858446 |
| | | | | accessory beta subunit | |
| UBE2T | ubiquitin conjugating | 2.073616696 | MARK1 | microtubule affinity | −1.944858446 |
| | enzyme E2 T | | | regulating kinase 1 | |
| CREB3L3 | CAMP responsive element | 2.070389328 | CHRNB1 | cholinergic receptor | −1.944858446 |
| | binding protein 3 like 3 | | | nicotinic beta 1 subunit | |
| CHRM4 | cholinergic receptor | 2.070389328 | CRYL1 | crystallin lambda 1 | −1.946419425 |
| | muscarinic 4 | | | | |
| SLC16A13 | solute carrier family 16 | 2.070389328 | TEC | tec protein tyrosine kinase | −1.947330641 |
| | member 13 | | | | |
| OLFML2B | olfactomedin like 2B | 2.070389328 | XKR6 | XK related 6 | −1.95031589 |
| CSNK1G1 | casein kinase 1 gamma 1 | 2.070389328 | ARC | activity-regulated | −1.953636949 |
| | | | | cytoskeleton-associated | |
| | | | | protein | |
| S100A14 | S100 calcium binding | 2.070389328 | WFDC10 | WAP four-disulfide core | −1.95419631 |
| | protein A14 | | | domain 10(Wfdc10) | |
| SMYD4 | SET and MYND domain | 2.070389328 | OLFR866 | olfactory receptor | −1.959768144 |
| | containing 4 | | | 866(Olfr866) | |
| CH25H | cholesterol 25- | 2.070389328 | WIPI2 | WD repeat domain, | −1.960171668 |
| | hydroxylase | | | phosphoinositide interacting | |
| | | | | 2 | |
| TEX2 | testis expressed 2 | 2.067875748 | OLFR948 | olfactory receptor | −1.963474124 |
| | | | | 948(Olfr948) | |
| SYN1 | synapsin I | 2.063429187 | CRTAM | cytotoxic and regulatory T- | −1.963474124 |
| | | | | cell molecule | |
| CYP3A13 | cytochrome P450, family | 2.060581758 | CCDC116 | coiled-coil domain | −1.963474124 |
| | 3, subfamily a, | | | containing 116 | |
| | polypeptide 13(Cyp3a13) | | | | |
| CBX8 | chromobox 8 | 2.060297534 | ALAS2 | 5′-aminolevulinate synthase | −1.963474124 |
| | | | | 2 | |
| TOR2A | torsin family 2 member A | 2.056535553 | SDC4 | syndecan 4 | −1.963474124 |
| E230025N22RIK | Riken cDNA | 2.053111336 | LENG1 | leukocyte receptor cluster | −1.963474124 |
| | E230025N22 | | | member 1 | |
| | gene(E230025N22Rik) | | | | |
| OLFR963 | olfactory receptor | 2.053111336 | TRIM65 | tripartite motif containing | −1.963474124 |
| | 963(Olfr963) | | | 65 | |
| OLFR694 | olfactory receptor | 2.053111336 | ADRA2B | adrenoceptor alpha 2B | −1.963474124 |
| | 694(Olfr694) | | | | |
| AKR1B8 | aldo-keto reductase | 2.053111336 | CPSF4 | cleavage and | −1.964016356 |
| | family 1, member | | | polyadenylation specific | |
| | B8(Akr1b8) | | | factor 4 | |
| UGDH | UDP-glucose 6- | 2.053111336 | LRCH1 | leucine rich repeats and | −1.966068313 |
| | dehydrogenase | | | calponin homology domain | |
| | | | | containing 1 | |
| CLPB | ClpB homolog, | 2.053111336 | CPXM1 | carboxypeptidase X (M14 | −1.96782195 |
| | mitochondrial AAA | | | family), member 1 | |
| | ATPase chaperonin | | | | |
| KLHDC9 | kelch domain containing | 2.053111336 | PARP6 | poly(ADP-ribose) | −1.968362498 |
| | 9 | | | polymerase family member | |
| | | | | 6 | |
| MCPH1 | microcephalin 1 | 2.051211057 | GTF3C2 | general transcription factor | −1.975687807 |
| | | | | IIIC subunit 2 | |
| IL2RA | interleukin 2 receptor | 2.049225103 | NEDD4L | neural precursor cell | −1.978518523 |
| | subunit alpha | | | expressed, developmentally | |
| | | | | down-regulated 4-like, E3 | |
| | | | | ubiquitin protein ligase | |
| CAR9 | carbonic anhydrase | 2.044394119 | DICER1 | dicer 1, ribonuclease III | −1.97959126 |
| | 9(Car9) | | | | |
| USP10 | ubiquitin specific | 2.044394119 | GBA2 | glucosylceramidase beta 2 | −1.980387638 |
| | peptidase 10 | | | | |
| FASTKD2 | FAST kinase domains 2 | 2.044394119 | OLFR1269 | olfactory receptor | −1.981852653 |
| | | | | 1269(Olfr1269) | |
| STRA13 | stimulated by retinoic | 2.044394119 | EAR10 | eosinophil-associated, | −1.981852653 |
| | acid 13 | | | ribonuclease A family, | |
| | | | | member 10(Ear10) | |
| HIST1H2AD | histone cluster 1, H2ad | 2.044111161 | ADAM5 | ADAM metallopeptidase | −1.981852653 |
| | | | | domain 5 (pseudogene) | |
| PLA1A | phospholipase A1 | 2.037157781 | MED1 | mediator complex subunit 1 | −1.981852653 |
| | member A | | | | |
| MCM3 | minichromosome | 2.036462274 | FGFRL1 | fibroblast growth factor | −1.981852653 |
| | maintenance complex | | | receptor-like 1 | |
| | component 3 | | | | |
| PIF1 | PIF1 5′-to-3′ DNA | 2.036094966 | EXTL1 | exostosin like | −1.981852653 |
| | helicase | | | glycosyltransferase 1 | |
| GALR1 | galanin receptor 1 | 2.03562391 | ZFHX3 | zinc finger homeobox 3 | −1.981852653 |
| DLD | dihydrolipoamide | 2.03562391 | FBXO30 | F-box protein 30 | −1.981852653 |
| | dehydrogenase | | | | |
| GGCX | gamma-glutamyl | 2.03562391 | RNF112 | ring finger protein 112 | −1.984681148 |
| | carboxylase | | | | |
| CEP68 | centrosomal protein 68 | 2.03562391 | PARP3 | poly(ADP-ribose) | −1.98599548 |
| | | | | polymerase family member | |
| | | | | 3 | |
| MMP11 | matrix metallopeptidase | 2.03562391 | AIRE | autoimmune regulator | −1.986410935 |
| | 11 | | | | |
| STMN1 | stathmin 1 | 2.033316653 | CYB561D1 | cytochrome b561 family | −1.987107951 |
| | | | | member D1 | |
| SLCO4A1 | solute carrier organic | 2.03217627 | TRAPPC5 | trafficking protein particle | −1.987269174 |
| | anion transporter family | | | complex 5 | |
| | member 4A1 | | | | |
| TIAL1 | TIA1 cytotoxic granule- | 2.02888965 | RFTN2 | raftlin family member 2 | −1.98749308 |
| | associated RNA binding | | | | |
| | protein-like 1 | | | | |
| 0610009B22RIK | Description Not Found | 2.017921908 | FRAT1 | frequently rearranged in | −1.999894159 |
| | | | | advanced T-cell lymphomas | |
| | | | | 1 | |
| GM1673 | predicted gene | 2.017921908 | DMC1 | DNA meiotic recombinase | −2 |
| | 1673(Gm1673) | | | 1 | |
| CCL26 | C-C motif chemokine | 2.017921908 | RIPK4 | receptor interacting | −2 |
| | ligand 26 | | | serine/threonine kinase 4 | |
| ZWILCH | zwilch kinetochore | 2.017921908 | PVR | poliovirus receptor | −2 |
| | protein | | | | |
| GABRA1 | gamma-aminobutyric acid | 2.017921908 | LPIN2 | lipin 2 | −2 |
| | type A receptor alpha1 | | | | |
| | subunit | | | | |
| ACP2 | acid phosphatase 2, | 2.017143376 | THAP2 | THAP domain containing 2 | −2 |
| | lysosomal | | | | |
| FAM131A | family with sequence | 2.013219985 | SHE | Src homology 2 domain | −2 |
| | similarity 131 member A | | | containing E | |
| PXMP4 | peroxisomal membrane | 2.012497517 | ARHGAP25 | Rho GTPase activating | −2.005618551 |
| | protein 4 | | | protein 25 | |
| CDC6 | cell division cycle 6 | 2.011166077 | CSF1R | colony stimulating factor 1 | −2.006350699 |
| | | | | receptor | |
| AXL | AXL receptor tyrosine | 2.008131619 | ZFP1 | ZFP1 zinc finger protein | −2.007904843 |
| | kinase | | | | |
| RBBP7 | RB binding protein 7, | 2.006746832 | SFN | stratifin | −2.008988783 |
| | chromatin remodeling | | | | |
| | factor | | | | |
| PABPC4 | poly(A) binding protein | 2.005260152 | COL17A1 | collagen type XVII alpha 1 | −2.010386372 |
| | cytoplasmic 4 | | | | |
| HIST1H2AK | histone cluster 1, H2ak | 2.003307679 | XKRX | XK related, X-linked | −2.0105696 |
| MTFMT | mitochondrial methionyl- | 2.001754595 | BRD8 | bromodomain containing 8 | −2.01346226 |
| | tRNA formyltransferase | | | | |
| ZFP449 | zinc finger protein | 2 | ZFP213 | zinc finger protein | −2.013532276 |
| | 449(Zfp449) | | | 213(Zfp213) | |
| D930020B18RIK | RIKEN cDNA | 2 | ZFY2 | zinc finger protein 2, Y- | −2.015657249 |
| | D930020B18 | | | linked(Zfy2) | |
| | gene(D930020B18Rik) | | | | |
| LCE1D | late cornified envelope | 2 | MAP3K3 | mitogen-activated protein | −2.01612652 |
| | 1D | | | kinase kinase kinase 3 | |
| UCN | urocortin | 2 | ZFP445 | zinc finger protein | −2.017921908 |
| | | | | 445(Zfp445) | |
| SYT4 | synaptotagmin 4 | 2 | MTAP7D3 | MAP7 domain containing | −2.017921908 |
| | | | | 3(Mtap7d3) | |
| GPR132 | G protein-coupled | 2 | TMPRSS11A | transmembrane protease, | −2.017921908 |
| | receptor 132 | | | serine 11A | |
| SDHD | succinate dehydrogenase | 2 | OLFM2 | olfactomedin 2 | −2.017921908 |
| | complex subunit D | | | | |
| PANK3 | pantothenate kinase 3 | 2 | GRM4 | glutamate metabotropic | −2.017921908 |
| | | | | receptor 4 | |
| SBSN | suprabasin | 1.99095486 | ONECUT2 | one cut homeobox 2 | −2.017921908 |
| WDR59 | WD repeat domain 59 | 1.989976974 | HNRNPH3 | heterogeneous nuclear | −2.017921908 |
| | | | | ribonucleoprotein H3 | |
| MTMR9 | myotubularin related | 1.987844644 | ZMYM5 | zinc finger MYM-type | −2.020204421 |
| | protein 9 | | | containing 5 | |
| IL15RA | interleukin 15 receptor | 1.985628881 | RAPGEF6 | Rap guanine nucleotide | −2.020953989 |
| | subunit alpha | | | exchange factor 6 | |
| RHBDF2 | rhomboid 5 homolog 2 | 1.984681148 | CD34 | CD34 molecule | −2.026714044 |
| NHLRC2 | NHL repeat containing 2 | 1.98375117 | ACVR2B | activin A receptor type 2B | −2.026714044 |
| NMRAL1 | NmrA-like family domain | 1.983370163 | RILP | Rab interacting lysosomal | −2.026800059 |
| | containing 1 | | | protein | |
| OLFR120 | olfactory receptor | 1.981852653 | EMR1 | Description Not Found | −2.031218731 |
| | 120(Olfr120) | | | | |
| OLFR1051 | olfactory receptor | 1.981852653 | DNAJA2 | DnaJ heat shock protein | −2.031291874 |
| | 1051(Olfr1051) | | | family (Hsp40) member A2 | |
| PCDHGA9 | protocadherin gamma | 1.981852653 | SEMA4B | semaphorin 4B | −2.031985281 |
| | subfamily A, 9 | | | | |
| FST | follistatin | 1.981852653 | 1700015E13RIK | Description Not Found | −2.03562391 |
| RECQL4 | RecQ like helicase 4 | 1.976611605 | RHOX1 | reproductive homeobox | −2.03562391 |
| | | | | 1(Rhox1) | |
| NFKBIL1 | NFKB inhibitor like 1 | 1.970969489 | TCP11 | t-complex 11 | −2.03562391 |
| TUBD1 | tubulin delta 1 | 1.964367355 | FBXW11 | F-box and WD repeat | −2.03562391 |
| | | | | domain containing 11 | |
| FSD1 | fibronectin type III and | 1.963474124 | ALX1 | ALX homeobox 1 | −2.03562391 |
| | SPRY domain containing | | | | |
| | 1 | | | | |
| GDF5 | growth differentiation | 1.963474124 | BST1 | bone marrow stromal cell | −2.03562391 |
| | factor 5 | | | antigen 1 | |
| TREML4 | triggering receptor | 1.963474124 | GPR83 | G protein-coupled receptor | −2.03562391 |
| | expressed on myeloid | | | 83 | |
| | cells like 4 | | | | |
| SORD | sorbitol dehydrogenase | 1.963474124 | RECK | reversion inducing cysteine | −2.036112118 |
| | | | | rich protein with kazal | |
| | | | | motifs | |
| HEBP1 | heme binding protein 1 | 1.963474124 | ABHD14B | abhydrolase domain | −2.040460993 |
| | | | | containing 14B | |
| KDELR2 | KDEL endoplasmic | 1.96155465 | GPRC6A | G protein-coupled receptor | −2.042122888 |
| | reticulum protein | | | class C group 6 member A | |
| | retention receptor 2 | | | | |
| TRPV4 | transient receptor | 1.958842675 | GRAMD3 | GRAM domain containing | −2.042296131 |
| | potential cation channel | | | 3 | |
| | subfamily V member 4 | | | | |
| ABHD5 | abhydrolase domain | 1.957389419 | IMPACT | impact RWD domain | −2.042436285 |
| | containing 5 | | | protein | |
| YOD1 | YOD1 deubiquitinase | 1.95419631 | TOP1 | topoisomerase (DNA) I | −2.044394119 |
| MAGOHB | mago homolog B, exon | 1.952932368 | NACC2 | NACC family member 2 | −2.044394119 |
| | junction complex core | | | | |
| | component | | | | |
| TSPAN2 | tetraspanin 2 | 1.95176103 | PKNOX1 | PBX/knotted 1 homeobox 1 | −2.045797958 |
| LDB3 | LIM domain binding 3 | 1.94850842 | TMEM79 | transmembrane protein 79 | −2.046628729 |
| 1700067P10RIK | Description Not Found | 1.944858446 | MYCBP2 | MYC binding protein 2, E3 | −2.047368853 |
| | | | | ubiquitin protein ligase | |
| 9530091C08RIK | Description Not Found | 1.944858446 | MAS1 | MAS1 proto-oncogene, G | −2.048055651 |
| | | | | protein-coupled receptor | |
| RHOJ | ras homolog family | 1.944858446 | GEMIN6 | gem nuclear organelle | −2.053111336 |
| | member J | | | associated protein 6 | |
| SFRP1 | secreted frizzled related | 1.944858446 | TMEM100 | transmembrane protein 100 | −2.053111336 |
| | protein 1 | | | | |
| XPNPEP2 | X-prolyl aminopeptidase | 1.944858446 | FOXI1 | forkhead box I1 | −2.053111336 |
| | (aminopeptidase P) 2, | | | | |
| | membrane-bound | | | | |
| RNASE4 | ribonuclease A family | 1.935459748 | OPLAH | 5-oxoprolinase (ATP- | −2.053111336 |
| | member 4 | | | hydrolysing) | |
| NAPSA | napsin A aspartic | 1.931586931 | BC094916 | Description Not Found | −2.058337935 |
| | peptidase | | | | |
| TIMM22 | translocase of inner | 1.931202999 | GZMM | granzyme M | −2.061193332 |
| | mitochondrial membrane | | | | |
| | 22 homolog (yeast) | | | | |
| MTCH2 | mitochondrial carrier 2 | 1.929774464 | RCOR2 | REST corepressor 2 | −2.06280495 |
| ADCK4 | aarF domain containing | 1.927921426 | NR2E1 | nuclear receptor subfamily | −2.06366268 |
| | kinase 4 | | | 2 group E member 1 | |
| PDSS1 | prenyl (decaprenyl) | 1.926245513 | NT5DC1 | 5′-nucleotidase domain | −2.065994119 |
| | diphosphate synthase, | | | containing 1 | |
| | subunit 1 | | | | |
| ZFP94 | zinc finger protein | 1.925999419 | SCN8A | sodium voltage-gated | −2.06750099 |
| | 94(Zfp94) | | | channel alpha subunit 8 | |
| FABP9 | fatty acid binding protein | 1.925999419 | CBX7 | chromobox 7 | −2.06750099 |
| | 9 | | | | |
| RNF170 | ring finger protein 170 | 1.925999419 | FHAD1 | forkhead associated | −2.068114527 |
| | | | | phosphopeptide binding | |
| | | | | domain 1 | |
| TLR3 | toll like receptor 3 | 1.925999419 | KCNQ3 | potassium voltage-gated | −2.068885643 |
| | | | | channel subfamily Q | |
| | | | | member 3 | |
| LIPH | lipase H | 1.925999419 | BC025920 | zinc finger protein | −2.070389328 |
| | | | | pseudogene(BC025920) | |
| PLEKHA7 | pleckstrin homology | 1.925999419 | FCGR1 | Fc receptor, IgG, high | −2.070389328 |
| | domain containing A7 | | | affinity I(Fcgr1) | |
| LXN | latexin | 1.9244606 | SYN3 | synapsin III | −2.070389328 |
| PPCS | phosphopantothenoylcyst | 1.92294738 | KLHL5 | kelch like family member 5 | −2.070389328 |
| | eine synthetase | | | | |
| BTRC | beta-transducin repeat | 1.92065845 | EDA2R | ectodysplasin A2 receptor | −2.070389328 |
| | containing E3 ubiquitin | | | | |
| | protein ligase | | | | |
| APIP | APAF1 interacting | 1.920326443 | STK38 | serine/threonine kinase 38 | −2.070389328 |
| | protein | | | | |
| ANK1 | ankyrin 1 | 1.916476644 | CDKN2D | cyclin dependent kinase | −2.072205467 |
| | | | | inhibitor 2D | |
| TOMM70A | translocase of outer | 1.913107017 | IL6ST | interleukin 6 signal | −2.072660321 |
| | mitochondrial membrane | | | transducer | |
| | 70 homolog A | | | | |
| | (yeast)(Tomm70a) | | | | |
| ABCB1B | ATP-binding cassette, | 1.908033945 | OLFR427 | olfactory receptor | −2.074318985 |
| | sub-family B | | | 427(Olfr427) | |
| | (MDR/TAP), member | | | | |
| | 1B(Abcb1b) | | | | |
| ACN9 | Description Not Found | 1.906890596 | BAIAP2 | BAIl associated protein 2 | −2.078951341 |
| DLX1AS | distal-less homeobox 1, | 1.906890596 | TIMP2 | TIMP metallopeptidase | −2.079805224 |
| | antisense(Dlx1as) | | | inhibitor 2 | |
| MRGPRD | MAS related GPR family | 1.906890596 | CDCP1 | CUB domain containing | −2.083991945 |
| | member D | | | protein 1 | |
| WDHD1 | WD repeat and HMG-box | 1.906890596 | RGS14 | regulator of G-protein | −2.084198537 |
| | DNA binding protein 1 | | | signaling 14 | |
| USP46 | ubiquitin specific | 1.906890596 | VASP | vasodilator-stimulated | −2.086359868 |
| | peptidase 46 | | | phosphoprotein | |
| PKN3 | protein kinase N3 | 1.906890596 | ZFP318 | zinc finger protein | −2.087462841 |
| | | | | 318(Zfp318) | |
| OSCAR | osteoclast associated, | 1.906890596 | PSG25 | pregnancy-specific | −2.087462841 |
| | immunoglobulin-like | | | glycoprotein 25(Psg25) | |
| | receptor | | | | |
| CDK2 | cyclin dependent kinase 2 | 1.906746727 | PDZD8 | PDZ domain containing 8 | −2.087462841 |
| TRIM62 | tripartite motif containing | 1.905520967 | DET1 | de-etiolated homolog 1 | −2.087462841 |
| | 62 | | | (Arabidopsis) | |
| SQLE | squalene epoxidase | 1.903767694 | CHST3 | carbohydrate | −2.087462841 |
| | | | | sulfotransferase 3 | |
| MCM10 | minichromosome | 1.89598378 | EHHADH | enoyl-CoA, hydratase/3- | −2.087462841 |
| | maintenance 10 | | | hydroxyacyl CoA | |
| | replication initiation | | | dehydrogenase | |
| | factor | | | | |
| CCDC90B | coiled-coil domain | 1.894803124 | FCGRT | Fc fragment of IgG receptor | −2.090735607 |
| | containing 90B | | | and transporter | |
| SPATS1 | spermatogenesis | 1.892848083 | CFP | complement factor | −2.09437407 |
| | associated serine rich 1 | | | properdin | |
| GPNMB | glycoprotein nmb | 1.891427809 | SOCS6 | suppressor of cytokine | −2.094638136 |
| | | | | signaling 6 | |
| MST1 | macrophage stimulating 1 | 1.88993148 | SYT11 | synaptotagmin 11 | −2.09592442 |
| LTB4R1 | leukotriene B4 receptor | 1.887644112 | MBTPS2 | membrane bound | −2.09592442 |
| | 1(Ltb4r1) | | | transcription factor | |
| | | | | peptidase, site 2 | |
| DNAJC5B | DnaJ heat shock protein | 1.887525271 | MEFV | Mediterranean fever | −2.097059135 |
| | family (Hsp40) member | | | | |
| | C5 beta | | | | |
| PCDHGC4 | protocadherin gamma | 1.887525271 | SRPK2 | SRSF protein kinase 2 | −2.10044313 |
| | subfamily C, 4 | | | | |
| HMX2 | H6 family homeobox 2 | 1.887525271 | DUSP16 | dual specificity phosphatase | −2.102740277 |
| | | | | 16 | |
| NDUFAB1 | NADH:ubiquinone | 1.887525271 | SLC6A7 | solute carrier family 6 | −2.103129681 |
| | oxidoreductase subunit | | | member 7 | |
| | AB1 | | | | |
| MGP | matrix Gla protein | 1.887525271 | HBB-B1 | hemoglobin, beta adult | −2.10433666 |
| | | | | major chain(Hbb-b1) | |
| ZKSCAN2 | zinc finger with KRAB | 1.887525271 | TNPO3 | transportin 3 | −2.10433666 |
| | and SCAN domains 2 | | | | |
| CCDC51 | coiled-coil domain | 1.887525271 | CSNK2B | casein kinase 2 beta | −2.10433666 |
| | containing 51 | | | | |
| CTSK | cathepsin K | 1.887525271 | BCAS1 | breast carcinoma amplified | −2.10433666 |
| | | | | sequence 1 | |
| PRDM9 | PR domain 9 | 1.887525271 | INO80 | INO80 complex subunit | −2.10433666 |
| C8A | complement component 8 | 1.887525271 | MPG | N-methylpurine DNA | −2.10433666 |
| | alpha subunit | | | glycosylase | |
| NEUROG1 | neurogenin 1 | 1.887082413 | FOXP1 | forkhead box P1 | −2.107557734 |
| NUSAP1 | nucleolar and spindle | 1.886951242 | USP21 | ubiquitin specific peptidase | −2.107658353 |
| | associated protein 1 | | | 21 | |
| LZIC | leucine zipper and | 1.877899051 | LIMS1 | LIM zinc finger domain | −2.112700133 |
| | CTNNBIP1 domain | | | containing 1 | |
| | containing | | | | |
| ZFP609 | zinc finger protein | 1.87774425 | FXYD1 | FXYD domain containing | −2.112700133 |
| | 609(Zfp609) | | | ion transport regulator 1 | |
| GPR87 | G protein-coupled | 1.87774425 | POU3F1 | POU class 3 homeobox 1 | −2.113574207 |
| | receptor 87 | | | | |
| GMPPB | GDP-mannose | 1.871523637 | OLFR591 | olfactory receptor | −2.114494844 |
| | pyrophosphorylase B | | | 591(Olfr591) | |
| TMEM115 | transmembrane protein | 1.870364796 | GRAMD4 | GRAM domain containing | −2.114673101 |
| | 115 | | | 4 | |
| DSN1 | DSN1 homolog, MIS12 | 1.868479018 | BCL2 | BCL2, apoptosis regulator | −2.115878669 |
| | kinetochore complex | | | | |
| | component | | | | |
| A530099J19RIK | Description Not Found | 1.867896464 | PELI3 | pellino E3 ubiquitin protein | −2.118915146 |
| | | | | ligase family member 3 | |
| 1700007K09RIK | Description Not Found | 1.867896464 | PPP1CB | protein phosphatase 1 | −2.119236221 |
| | | | | catalytic subunit beta | |
| 1810043G02RIK | Description Not Found | 1.867896464 | TFF2 | trefoil factor 2 | −2.121015401 |
| UCHL1 | ubiquitin C-terminal | 1.867896464 | GCA | grancalcin | −2.121015401 |
| | hydrolase L1 | | | | |
| PTCH2 | patched 2 | 1.867896464 | LYL1 | LYL1, basic helix-loop- | −2.121015401 |
| | | | | helix family member | |
| APBB3 | amyloid beta precursor | 1.867896464 | ATG4B | autophagy related 4B | −2.121015401 |
| | protein binding family B | | | cysteine peptidase | |
| | member 3 | | | | |
| PTER | phosphotriesterase related | 1.867896464 | CCDC102A | coiled-coil domain | −2.121015401 |
| | | | | containing 102A | |
| PRKCE | protein kinase C epsilon | 1.867896464 | ATP2A1 | ATPase | −2.121015401 |
| | | | | sarcoplasmic/endoplasmic | |
| | | | | reticulum Ca2+ transporting | |
| | | | | 1 | |
| PLEKHM3 | pleckstrin homology | 1.867896464 | TERF2 | telomeric repeat binding | −2.123585568 |
| | domain containing M3 | | | factor 2 | |
| HIST1H4C | histone cluster 1, H4c | 1.867896464 | LCN5 | lipocalin 5(Lcn5) | −2.124432612 |
| PLS3 | plastin 3 | 1.867896464 | TM6SF1 | transmembrane 6 | −2.124533495 |
| | | | | superfamily member 1 | |
| DUSP4 | dual specificity | 1.867686654 | SSBP2 | single stranded DNA | −2.129283017 |
| | phosphatase 4 | | | binding protein 2 | |
| SCLY | selenocysteine lyase | 1.862802277 | KRTAP6-2 | keratin associated protein 6- | −2.137503524 |
| | | | | 2 | |
| RPRD1A | regulation of nuclear pre- | 1.861777838 | CRHBP | corticotropin releasing | −2.137503524 |
| | mRNA domain | | | hormone binding protein | |
| | containing 1A | | | | |
| CCRL2 | C-C motif chemokine | 1.86175579 | TOPBP1 | topoisomerase (DNA) II | −2.137503524 |
| | receptor like 2 | | | binding protein 1 | |
| CCT7 | chaperonin containing | 1.861636037 | SLC35A3 | solute carrier family 35 | −2.137503524 |
| | TCP1 subunit 7 | | | member A3 | |
| ZFP217 | zinc finger protein | 1.861097096 | CACNB4 | calcium voltage-gated | −2.137503524 |
| | 217(Zfp217) | | | channel auxiliary subunit | |
| | | | | beta 4 | |
| ACTN4 | actinin alpha 4 | 1.859689938 | TASP1 | taspase 1 | −2.137503524 |
| KCNA3 | potassium voltage-gated | 1.859135363 | HMBOX1 | homeobox containing 1 | −2.145313833 |
| | channel subfamily A | | | | |
| | member 3 | | | | |
| CUL7 | cullin 7 | 1.858597911 | ZFP62 | ZFP62 zinc finger protein | −2.145677455 |
| LRRC59 | leucine rich repeat | 1.857543219 | PCDHB4 | protocadherin beta 4 | −2.148666128 |
| | containing 59 | | | | |
| PHTF2 | putative homeodomain | 1.855602651 | SLC35F3 | solute carrier family 35 | −2.15120644 |
| | transcription factor 2 | | | member F3 | |
| KDELC1 | KDEL motif containing 1 | 1.852556218 | AW549877 | expressed sequence | −2.151324826 |
| | | | | AW549877(AW549877) | |
| SEC24D | SEC24 homolog D, | 1.8483841 | GIMAP9 | GTPase, IMAP family | −2.152400921 |
| | COPII coat complex | | | member 9(Gimap9) | |
| | component | | | | |
| OLFR222 | olfactory receptor | 1.847996907 | ZFP329 | zinc finger protein | −2.153805336 |
| | 222(Olfr222) | | | 329(Zfp329) | |
| OLFR118 | olfactory receptor | 1.847996907 | KRT74 | keratin 74 | −2.153805336 |
| | 118(Olfr118) | | | | |
| CASKIN2 | CASK interacting protein | 1.847996907 | REG3A | regenerating family | −2.153805336 |
| | 2 | | | member 3 alpha | |
| TPK1 | thiamin | 1.847996907 | RAB4A | RAB4A, member RAS | −2.154308231 |
| | pyrophosphokinase 1 | | | oncogene family | |
| NOL3 | nucleolar protein 3 | 1.847996907 | CECR5 | cat eye syndrome | −2.155682653 |
| | | | | chromosome region, | |
| | | | | candidate 5 | |
| UBA6 | ubiquitin like modifier | 1.847388943 | ESM1 | endothelial cell specific | −2.157156463 |
| | activating enzyme 6 | | | molecule 1 | |
| RAVER1 | ribonucleoprotein, PTB | 1.846151947 | HS6ST1 | heparan sulfate 6-O- | −2.164820712 |
| | binding 1 | | | sulfotransferase 1 | |
| NAT10 | N-acetyltransferase 10 | 1.843300131 | DDB2 | damage specific DNA | −2.168338824 |
| | | | | binding protein 2 | |
| HIST1H3H | histone cluster 1, H3h | 1.842055889 | 5430435G22RIK | Description Not Found | −2.169925001 |
| SNX8 | sorting nexin 8 | 1.840985134 | ALOX12B | arachidonate 12- | −2.169925001 |
| | | | | lipoxygenase, 12R type | |
| POLR3K | polymerase (RNA) III | 1.839538616 | SLC34A3 | solute carrier family 34 | −2.169925001 |
| | subunit K | | | member 3 | |
| WDR55 | WD repeat domain 55 | 1.835957408 | TNS4 | tensin 4 | −2.169925001 |
| WDR93 | WD repeat domain 93 | 1.830541464 | CANX | calnexin | −2.169925001 |
| PLSCR1 | phospholipid scramblase | 1.828635636 | BET1 | Bet1 golgi vesicular | −2.169925001 |
| | 1 | | | membrane trafficking | |
| | | | | protein | |
| ARL6 | ADP ribosylation factor | 1.827819025 | BEST2 | bestrophin 2 | −2.169925001 |
| | like GTPase 6 | | | | |
| NOL9 | nucleolar protein 9 | 1.827819025 | USP28 | ubiquitin specific peptidase | −2.172998154 |
| | | | | 28 | |
| PNKD | paroxysmal | 1.827819025 | PDE4B | phosphodiesterase 4B | −2.173614018 |
| | nonkinesigenic dyskinesia | | | | |
| TMEM139 | transmembrane protein | 1.827819025 | CNOT4 | CCR4-NOT transcription | −2.177917792 |
| | 139 | | | complex subunit 4 | |
| ASPH | aspartate beta- | 1.827819025 | NECAP1 | NECAP endocytosis | −2.178043245 |
| | hydroxylase | | | associated 1 | |
| LZTFL1 | leucine zipper | 1.827819025 | JUN | Jun proto-oncogene, AP-1 | −2.178565309 |
| | transcription factor like 1 | | | transcription factor subunit | |
| RHEBL1 | Ras homolog enriched in | 1.827819025 | SLC10A7 | solute carrier family 10 | −2.17990909 |
| | brain like 1 | | | member 7 | |
| CHCHD5 | coiled-coil-helix-coiled- | 1.82552849 | IL17A | interleukin 17A | −2.181702586 |
| | coil-helix domain | | | | |
| | containing 5 | | | | |
| GPD2 | glycerol-3-phosphate | 1.824148697 | ERICH1 | glutamate rich 1 | −2.182286216 |
| | dehydrogenase 2 | | | | |
| STK39 | serine/threonine kinase 39 | 1.823608879 | HN1L | hematological and | −2.185866545 |
| | | | | neurological expressed 1- | |
| | | | | like | |
| MAGED2 | MAGE family member | 1.820863253 | SLFNL1 | schlafen like 1 | −2.185866545 |
| | D2 | | | | |
| TBC1D9B | TBC1 domain family | 1.813219568 | MYOD1 | myogenic differentiation 1 | −2.185866545 |
| | member 9B | | | | |
| LSS | lanosterol synthase (2,3- | 1.809540228 | TRIM35 | tripartite motif containing | −2.185866545 |
| | oxidosqualene-lanosterol | | | 35 | |
| | cyclase) | | | | |
| OLFR859 | olfactory receptor | 1.807354922 | CHRNE | cholinergic receptor | −2.186397884 |
| | 859(Olfr859) | | | nicotinic epsilon subunit | |
| OLFR1225 | olfactory receptor | 1.807354922 | PHF21A | PHD finger protein 21A | −2.190943197 |
| | 1225(Olfr1225) | | | | |
| IFNA11 | interferon alpha | 1.807354922 | HIST1H2AE | histone cluster 1, H2ae | −2.196698179 |
| | 11(Ifna11) | | | | |
| ARG1 | arginase 1 | 1.807354922 | SATB1 | SATB homeobox 1 | −2.198659952 |
| ASCL3 | achaete-scute family | 1.807354922 | LCN8 | lipocalin 8 | −2.201633861 |
| | bHLH transcription factor | | | | |
| | 3 | | | | |
| AGA | aspartylglucosaminidase | 1.807354922 | ABCG5 | ATP binding cassette | −2.201633861 |
| | | | | subfamily G member 5 | |
| MAP3K12 | mitogen-activated protein | 1.806530545 | KRBA1 | KRAB-A domain | −2.202959029 |
| | kinase kinase kinase 12 | | | containing 1 | |
| COMMD10 | COMM domain | 1.802771724 | CD274 | CD274 molecule | −2.206081393 |
| | containing 10 | | | | |
| STYX | serine/threonine/tyrosine | 1.801251483 | DYRK2 | dual specificity tyrosine | −2.206730511 |
| | interacting protein | | | phosphorylation regulated | |
| | | | | kinase 2 | |
| EPHA6 | EPH receptor A6 | 1.797583147 | ZFP292 | zinc finger protein | −2.209453366 |
| | | | | 292(Zfp292) | |
| SERPINA3F | serine (or cysteine) | 1.794445043 | PRX | periaxin | −2.209453366 |
| | peptidase inhibitor, clade | | | | |
| | A, member 3F(Serpina3f) | | | | |
| PUS10 | pseudouridylate synthase | 1.791814071 | SPAG1 | sperm associated antigen 1 | −2.209453366 |
| | 10 | | | | |
| RASL12 | RAS like family 12 | 1.791652715 | ASGR2 | asialoglycoprotein receptor | −2.209784456 |
| | | | | 2 | |
| MRPL51 | mitochondrial ribosomal | 1.787631232 | PTEN | phosphatase and tensin | −2.215013513 |
| | protein L51 | | | homolog | |
| OLFR1306 | olfactory receptor | 1.786596362 | IL1A | interleukin 1 alpha | −2.217230716 |
| | 1306(Olfr1306) | | | | |
| BCL2A1C | B cell | 1.786596362 | TPCN2 | two pore segment channel 2 | −2.217230716 |
| | leukemia/lymphoma 2 | | | | |
| | related protein | | | | |
| | Alc(Bcl2a1c) | | | | |
| HOXD1 | homeobox D1 | 1.786596362 | IKBKB | inhibitor of kappa light | −2.217230716 |
| | | | | polypeptide gene enhancer | |
| | | | | in B-cells, kinase beta | |
| MEMO1 | mediator of cell motility 1 | 1.786596362 | ST6GAL1 | ST6 beta-galactoside alpha- | −2.218342351 |
| | | | | 2,6-sialyltransferase 1 | |
| ARCN1 | archain 1 | 1.786596362 | TMEM161A | transmembrane protein | −2.232660757 |
| | | | | 161A | |
| NUDT10 | nudix hydrolase 10 | 1.786596362 | STK32B | serine/threonine kinase 32B | −2.232660757 |
| SLC4A4 | solute carrier family 4 | 1.786596362 | CHST14 | carbohydrate | −2.232660757 |
| | member 4 | | | sulfotransferase 14 | |
| DHRS4 | dehydrogenase/reductase | 1.786596362 | AQP3 | aquaporin 3 (Gill blood | −2.232660757 |
| | 4 | | | group) | |
| TOM1 | target of myb1 membrane | 1.786596362 | RASSF3 | Ras association domain | −2.233505898 |
| | trafficking protein | | | family member 3 | |
| TST | thiosulfate | 1.786596362 | OTUD7B | OTU deubiquitinase 7B | −2.242923867 |
| | sulfurtransferase | | | | |
| RIPK2 | receptor interacting | 1.784428584 | AP3M2 | adaptor related protein | −2.247481244 |
| | serine/threonine kinase 2 | | | complex 3 mu 2 subunit | |
| NAIP2 | NLR family, apoptosis | 1.780351745 | PSMA6 | proteasome subunit alpha 6 | −2.247927513 |
| | inhibitory protein | | | | |
| | 2(Naip2) | | | | |
| OLFR133 | olfactory receptor | 1.77946628 | PRCC | papillary renal cell | −2.247927513 |
| | 133(Olfr133) | | | carcinoma (translocation- | |
| | | | | associated) | |
| NBR1 | NBR1, autophagy cargo | 1.776995396 | ZFP688 | zinc finger protein | −2.262218541 |
| | receptor | | | 688(Zfp688) | |
| GLIS1 | GLIS family zinc finger 1 | 1.776512203 | DOCK11 | dedicator of cytokinesis 11 | −2.262218541 |
| SLC35A2 | solute carrier family 35 | 1.776232819 | PLA2G4F | phospholipase A2 group | −2.263034406 |
| | member A2 | | | IVF | |
| AU022252 | expressed sequence | 1.774559318 | MYPN | myopalladin | −2.263034406 |
| | AU022252(AU022252) | | | | |
| OLFR64 | olfactory receptor | 1.773991786 | FRS2 | fibroblast growth factor | −2.263034406 |
| | 64(Olfr64) | | | receptor substrate 2 | |
| PPAPDC2 | Description Not Found | 1.771983065 | STARD6 | StAR related lipid transfer | −2.263034406 |
| | | | | domain containing 6 | |
| DIS3 | DIS3 homolog, exosome | 1.771375295 | WSCD2 | WSC domain containing 2 | −2.270653766 |
| | endoribonuclease and 3′- | | | | |
| | 5′ exoribonuclease | | | | |
| 4931440F15RIK | Description Not Found | 1.770829046 | TLE1 | transducin like enhancer of | −2.272631746 |
| | | | | split 1 | |
| ZFP771 | zinc finger protein | 1.77019569 | HDHD3 | haloacid dehalogenase like | −2.272966802 |
| | 771(Zfp771) | | | hydrolase domain | |
| | | | | containing 3 | |
| HMBS | hydroxymethylbilane | 1.769676967 | 1700029J07RIK | Description Not Found | −2.277984747 |
| | synthase | | | | |
| RCC1 | regulator of chromosome | 1.768267605 | CLEC2D | C-type lectin domain family | −2.277984747 |
| | condensation 1 | | | 2 member D | |
| SPAG5 | sperm associated antigen | 1.767980257 | PPM1G | protein phosphatase, | −2.277984747 |
| | 5 | | | Mg2+/Mn2+ dependent 1G | |
| TSPAN31 | tetraspanin 31 | 1.767626782 | CDKN1B | cyclin dependent kinase | −2.280970508 |
| | | | | inhibitor 1B | |
| PCDHGB8 | protocadherin gamma | 1.765534746 | OASL1 | 2′-5′ oligoadenylate | −2.28169825 |
| | subfamily B, 8(Pcdhgb8) | | | synthetase-like 1(Oasl1) | |
| PRL2B1 | prolactin family 2, | 1.765534746 | G0S2 | G0/G1 switch 2 | −2.282045463 |
| | subfamily b, member | | | | |
| | 1(Prl2b1) | | | | |
| OBOX5 | oocyte specific homeobox | 1.765534746 | TMEM17 | transmembrane protein 17 | −2.285402219 |
| | 5(Obox5) | | | | |
| PIK3R3 | phosphoinositide-3-kinase | 1.765534746 | BLVRB | biliverdin reductase B | −2.290619427 |
| | regulatory subunit 3 | | | | |
| MAP3K4 | mitogen-activated protein | 1.765534746 | GOSR1 | golgi SNAP receptor | −2.290897209 |
| | kinase kinase kinase 4 | | | complex member 1 | |
| LRRC30 | leucine rich repeat | 1.765534746 | ZFP26 | zinc finger protein | −2.292781749 |
| | containing 30 | | | 26(Zfp26) | |
| EN2 | engrailed homeobox 2 | 1.765534746 | CXCL2 | C-X-C motif chemokine | −2.292781749 |
| | | | | ligand 2 | |
| HOOK3 | hook microtubule- | 1.765534746 | SNX7 | sorting nexin 7 | −2.292781749 |
| | tethering protein 3 | | | | |
| MYO9A | myosin IXA | 1.765534746 | ZDHHC23 | zinc finger DHHC-type | −2.292781749 |
| | | | | containing 23 | |
| STX7 | syntaxin 7 | 1.765060364 | GALNT6 | polypeptide N- | −2.292781749 |
| | | | | acetylgalactosaminyl- | |
| | | | | transferase 6 | |
| ATM | ATM serine/threonine | 1.763504031 | AMPD1 | adenosine monophosphate | −2.297844157 |
| | kinase | | | deaminase 1 | |
| KCNK6 | potassium two pore | 1.763385753 | GIMAP5 | GTPase, IMAP family | −2.303246615 |
| | domain channel | | | member 5 | |
| | subfamily K member 6 | | | | |
| PQLC3 | PQ loop repeat containing | 1.759954577 | ATP5F1 | ATP synthase, H+ | −2.305399163 |
| | 3 | | | transporting, mitochondrial | |
| | | | | Fo complex subunit B1 | |
| KIFAP3 | kinesin associated protein | 1.758843168 | LHFPL2 | lipoma HMGIC fusion | −2.307428525 |
| | 3 | | | partner-like 2 | |
| E2F4 | E2F transcription factor 4 | 1.757752886 | KIF1B | kinesin family member 1B | −2.313231129 |
| ETV5 | ETS variant 5 | 1.757709335 | TLE6 | transducin like enhancer of | −2.321928095 |
| | | | | split 6 | |
| GTF2E2 | general transcription | 1.75666387 | SHF | Src homology 2 domain | −2.330691998 |
| | factor IIE subunit 2 | | | containing F | |
| GPR150 | G protein-coupled | 1.75547927 | NGFR | nerve growth factor | −2.331438521 |
| | receptor 150 | | | receptor | |
| E130308A19RIK | RIKEN cDNA | 1.754887502 | KLRA4 | killer cell lectin-like | −2.334485632 |
| | E130308A19 | | | receptor, subfamily A, | |
| | gene(E130308A19Rik) | | | member 4(Klra4) | |
| DPYSL4 | dihydropyrimidinase like | 1.754887502 | ITGAE | integrin subunit alpha E | −2.335948972 |
| | 4 | | | | |
| FNBP1 | formin binding protein 1 | 1.75468902 | PQLC2 | PQ loop repeat containing 2 | −2.336141568 |
| TMOD4 | tropomodulin 4 | 1.754064107 | KLRB1A | killer cell lectin-like | −2.336283388 |
| | | | | receptor subfamily B | |
| | | | | member 1A(Klrb1a) | |
| ERLIN1 | ER lipid raft associated 1 | 1.751154691 | IRF9 | interferon regulatory factor | −2.336308285 |
| | | | | 9 | |
| ENOPH1 | enolase-phosphatase 1 | 1.748447442 | GATA3 | GATA binding protein 3 | −2.338971433 |
| RAB31 | RAB31, member RAS | 1.746215332 | RSAD2 | radical S-adenosyl | −2.33997952 |
| | oncogene family | | | methionine domain | |
| | | | | containing 2 | |
| HOXA6 | homeobox A6 | 1.745184623 | RNF215 | ring finger protein 215 | −2.341976415 |
| TAS2R126 | taste receptor, type 2, | 1.744161096 | IL7R | interleukin 7 receptor | −2.343395577 |
| | member 126(Tas2r126) | | | | |
| AGXT2 | alanine--glyoxylate | 1.744161096 | ACP5 | acid phosphatase 5, tartrate | −2.345270806 |
| | aminotransferase 2 | | | resistant | |
| STK32C | serine/threonine kinase | 1.744161096 | STYXL1 | serine/threonine/tyrosine | −2.346956889 |
| | 32C | | | interacting-like 1 | |
| P2RY2 | purinergic receptor P2Y2 | 1.744161096 | NOXO1 | NADPH oxidase organizer | −2.35030956 |
| | | | | 1 | |
| NWD1 | NACHT and WD repeat | 1.744161096 | IGFALS | insulin like growth factor | −2.358664554 |
| | domain containing 1 | | | binding protein acid labile | |
| | | | | subunit | |
| UQCRQ | ubiquinol-cytochrome c | 1.744161096 | STIM1 | stromal interaction | −2.359335599 |
| | reductase complex III | | | molecule 1 | |
| | subunit VII | | | | |
| PPP1R3A | protein phosphatase 1 | 1.744161096 | TMEM186 | transmembrane protein 186 | −2.361030771 |
| | regulatory subunit 3A | | | | |
| GOLT1A | golgi transport 1A | 1.744161096 | OLFR1043 | olfactory receptor | −2.364572432 |
| | | | | 1043(Olfr1043) | |
| EZH1 | enhancer of zeste 1 | 1.744161096 | D8ERTD82E | DNA segment, Chr 8, | −2.364572432 |
| | polycomb repressive | | | ERATO Doi 82, | |
| | complex 2 subunit | | | expressed(D8Ertd82e) | |
| MTHFD2 | methylenetetrahydrofolate | 1.744154314 | MYOG | myogenin | −2.364572432 |
| | dehydrogenase (NADP+ | | | | |
| | dependent) 2, | | | | |
| | methenyltetrahydrofolate | | | | |
| | cyclohydrolase | | | | |
| PGRMC1 | progesterone receptor | 1.742545062 | NCLN | nicalin | −2.364572432 |
| | membrane component 1 | | | | |
| DNAJB12 | DnaJ heat shock protein | 1.741863621 | MTSS1 | metastasis suppressor 1 | −2.364572432 |
| | family (Hsp40) member | | | | |
| | B12 | | | | |
| DNAJC11 | DnaJ heat shock protein | 1.738767837 | TRMU | tRNA 5- | −2.364572432 |
| | family (Hsp40) member | | | methylaminomethyl-2- | |
| | C11 | | | thiouridylate | |
| | | | | methyltransferase | |
| TOMM6 | translocase of outer | 1.738448709 | EMILIN2 | elastin microfibril interfacer | −2.369119767 |
| | mitochondrial membrane | | | 2 | |
| | 6 | | | | |
| RPS6KL1 | ribosomal protein S6 | 1.738393453 | MPV17L | MPV17 mitochondrial inner | −2.371558863 |
| | kinase like 1 | | | membrane protein like | |
| CDC73 | cell division cycle 73 | 1.73665741 | WWC2 | WW and C2 domain | −2.371558863 |
| | | | | containing 2 | |
| NDC80 | NDC80, kinetochore | 1.732078892 | TMEM178 | transmembrane protein | −2.374005585 |
| | complex component | | | 178(Tmem178) | |
| TACC3 | transforming acidic | 1.731372884 | TPCN1 | two pore segment channel 1 | −2.375232208 |
| | coiled-coil containing | | | | |
| | protein 3 | | | | |
| CPSF3 | cleavage and | 1.727926568 | LRRC45 | leucine rich repeat | −2.377207351 |
| | polyadenylation specific | | | containing 45 | |
| | factor 3 | | | | |
| ARID3A | AT-rich interaction | 1.726471722 | 1110059G10RIK | Description Not Found | −2.377915929 |
| | domain 3A | | | | |
| LLPH | LLP homolog, long-term | 1.726107859 | MCOLN2 | mucolipin 2 | −2.378511623 |
| | synaptic facilitation | | | | |
| PCNA | proliferating cell nuclear | 1.725441599 | DDX58 | DEXD/H-box helicase 58 | −2.378511623 |
| | antigen | | | | |
| GJC2 | gap junction protein | 1.722978517 | H2-OA | histocompatibility 2, O | −2.382329516 |
| | gamma 2 | | | region alpha locus(H2-Oa) | |
| OLFR373 | olfactory receptor | 1.722466024 | RARG | retinoic acid receptor | −2.388827772 |
| | 373(Olfr373) | | | gamma | |
| H2-T24 | histocompatibility 2, T | 1.722466024 | SERPINB1A | serine (or cysteine) | −2.392317423 |
| | region locus 24(H2-T24) | | | peptidase inhibitor, clade B, | |
| | | | | member la(Serpinb1a) | |
| AKAP7 | A-kinase anchoring | 1.722466024 | GHRL | ghrelin/obestatin | −2.392317423 |
| | protein 7 | | | prepropeptide | |
| NDUFB7 | NADH:ubiquinone | 1.722466024 | ZMAT4 | zinc finger matrin-type 4 | −2.392317423 |
| | oxidoreductase subunit | | | | |
| | B7 | | | | |
| PRR11 | proline rich 11 | 1.722466024 | BTBD6 | BTB domain containing 6 | −2.392897478 |
| TJP1 | tight junction protein 1 | 1.722466024 | KLRA16 | killer cell lectin-like | −2.394534969 |
| | | | | receptor, subfamily A, | |
| | | | | member 16(Klra16) | |
| S100A3 | S100 calcium binding | 1.722466024 | EPS15L1 | epidermal growth factor | −2.397012831 |
| | protein A3 | | | receptor pathway substrate | |
| | | | | 15 like 1 | |
| KRT78 | keratin 78 | 1.718729711 | VCPIP1 | valosin containing protein | −2.397303585 |
| | | | | interacting protein 1 | |
| GMDS | GDP-mannose 4,6- | 1.717904741 | RRP7A | ribosomal RNA processing | −2.404992223 |
| | dehydratase | | | 7 homolog A | |
| PDGFB | platelet derived growth | 1.714400534 | IL1B | interleukin 1 beta | −2.40599236 |
| | factor subunit B | | | | |
| SLC36A1 | solute carrier family 36 | 1.714297338 | NAT14 | N-acetyltransferase 14 | −2.40599236 |
| | member 1 | | | (putative) | |
| RSU1 | Ras suppressor protein 1 | 1.712647036 | SLC40A1 | solute carrier family 40 | −2.40599236 |
| | | | | member 1 | |
| STX12 | syntaxin 12 | 1.711911478 | RAB37 | RAB37, member RAS | −2.40599236 |
| | | | | oncogene family | |
| SLC25A34 | solute carrier family 25 | 1.711494907 | IL 17RA | interleukin 17 receptor A | −2.40599236 |
| | member 34 | | | | |
| AFG3L2 | AFG3 like matrix AAA | 1.711057 | BACE1 | beta-secretase 1 | −2.40599236 |
| | peptidase subunit 2 | | | | |
| RPL24 | ribosomal protein L24 | 1.709193708 | CTNS | cystinosin, lysosomal | −2.40599236 |
| | | | | cystine transporter | |
| UBE3C | ubiquitin protein ligase | 1.708789682 | IFIT3 | interferon induced protein | −2.411404504 |
| | E3C | | | with tetratricopeptide | |
| | | | | repeats 3 | |
| CAR12 | carbonic anhydrase | 1.70867626 | ZFYVE21 | zinc finger FYVE-type | −2.412378292 |
| | 12(Car12) | | | containing 21 | |
| ZFP207 | zinc finger protein | 1.707603009 | 1700016D06RIK | Description Not Found | −2.419538892 |
| | 207(Zfp207) | | | | |
| XIST | X inactive specific | 1.706065607 | STK25 | serine/threonine kinase 25 | −2.419538892 |
| | transcript (non-protein | | | | |
| | coding) | | | | |
| NCAPD2 | non-SMC condensin I | 1.705012178 | PLEKHJ1 | pleckstrin homology | −2.419538892 |
| | complex subunit D2 | | | domain containing J1 | |
| ZSWIM2 | zinc finger SWIM-type | 1.704802998 | TGIF2 | TGFB induced factor | −2.419538892 |
| | containing 2 | | | homeobox 2 | |
| CASP1 | caspase 1 | 1.70065942 | SLC25A29 | solute carrier family 25 | −2.419538892 |
| | | | | member 29 | |
| OLFR701 | olfactory receptor | 1.700439718 | DAPL1 | death associated protein like | −2.419661316 |
| | 701(Olfr701) | | | 1 | |
| CBLC | Cbl proto-oncogene C | 1.700439718 | P2RX4 | purinergic receptor P2X 4 | −2.425748008 |
| HIST1H2AC | histone cluster 1, H2ac | 1.700439718 | 1700001O22RIK | Description Not Found | −2.426264755 |
| EPHA10 | EPH receptor A10 | 1.700439718 | C9 | complement component 9 | −2.429615964 |
| NDUFC2 | NADH:ubiquinone | 1.700439718 | KLF13 | Kruppel like factor 13 | −2.430628023 |
| | oxidoreductase subunit | | | | |
| | C2 | | | | |
| DLG1 | discs large MAGUK | 1.700439718 | GADD45A | growth arrest and DNA | −2.432591239 |
| | scaffold protein 1 | | | damage inducible alpha | |
| SCN10A | sodium voltage-gated | 1.700439718 | OLFR788 | olfactory receptor | −2.432959407 |
| | channel alpha subunit 10 | | | 788(Olfr788) | |
| RGL3 | ral guanine nucleotide | 1.700439718 | FADS6 | fatty acid desaturase 6 | −2.432959407 |
| | dissociation stimulator | | | | |
| | like 3 | | | | |
| TMCO3 | transmembrane and | 1.700439718 | CHCHD2 | coiled-coil-helix-coiled- | −2.432959407 |
| | coiled-coil domains 3 | | | coil-helix domain | |
| | | | | containing 2 | |
| BCL2L14 | BCL2 like 14 | 1.700439718 | MPPE1 | metallophosphoesterase 1 | −2.432959407 |
| THOP1 | thimet oligopeptidase 1 | 1.700290033 | CHAC1 | ChaC glutathione specific | −2.432959407 |
| | | | | gamma- | |
| | | | | glutamylcyclotransferase 1 | |
| MTIF3 | mitochondrial | 1.698305331 | 2310011J03RIK | Description Not Found | −2.435017448 |
| | translational initiation | | | | |
| | factor 3 | | | | |
| XDH | xanthine dehydrogenase | 1.697717724 | LRSAM1 | leucine rich repeat and | −2.437473925 |
| | | | | sterile alpha motif | |
| | | | | containing 1 | |
| ANXA9 | annexin A9 | 1.697184071 | SIRPA | signal regulatory protein | −2.443125132 |
| | | | | alpha | |
| OLFR1502 | olfactory receptor | 1.694046727 | CYP24A1 | cytochrome P450 family 24 | −2.44625623 |
| | 1502(Olfr1502) | | | subfamily A member 1 | |
| HCFC2 | host cell factor C2 | 1.693780609 | NQO1 | NAD(P)H quinone | −2.44625623 |
| | | | | dehydrogenase 1 | |
| DIDO1 | death inducer-obliterator | 1.693596948 | HRH4 | histamine receptor H4 | −2.44625623 |
| | 1 | | | | |
| PGAM1 | phosphoglycerate mutase | 1.689846917 | NUDCD1 | NudC domain containing 1 | −2.44625623 |
| | 1 | | | | |
| RASGEFIC | RasGEF domain family | 1.689299161 | CCND1 | cyclin D1 | −2.447924527 |
| | member 1C | | | | |
| SLC25A42 | solute carrier family 25 | 1.686774817 | ADAM22 | ADAM metallopeptidase | −2.452858965 |
| | member 42 | | | domain 22 | |
| CPT2 | carnitine | 1.686364794 | MDK | midkine (neurite growth- | −2.456149035 |
| | palmitoyltransferase 2 | | | promoting factor 2) | |
| MAD2L1 | MAD2 mitotic arrest | 1.686161103 | STX1A | syntaxin 1A | −2.456729828 |
| | deficient-like 1 (yeast) | | | | |
| NQO2 | NAD(P)H quinone | 1.685558757 | HEMK1 | HemK methyltransferase | −2.459431619 |
| | dehydrogenase 2 | | | family member 1 | |
| HIP1R | huntingtin interacting | 1.685473307 | B4GALT7 | beta-1,4- | −2.459431619 |
| | protein 1 related | | | galactosyltransferase 7 | |
| ALOX12E | arachidonate | 1.684373244 | ASXL2 | additional sex combs like 2, | −2.459431619 |
| | lipoxygenase, | | | transcriptional regulator | |
| | epidermal(Alox12e) | | | | |
| LMAN1 | lectin, mannose binding 1 | 1.683514205 | TLR7 | toll like receptor 7 | −2.46052038 |
| ASB3 | ankyrin repeat and SOCS | 1.680142991 | TDP1 | tyrosyl-DNA | −2.464461869 |
| | box containing 3 | | | phosphodiesterase 1 | |
| XKR5 | XK related 5 | 1.679254438 | 1700025G04RIK | Description Not Found | −2.469303076 |
| ZFP235 | zinc finger protein | 1.678071905 | SLC16A6 | solute carrier family 16 | −2.471045434 |
| | 235(Zfp235) | | | member 6 | |
| OLFR971 | olfactory receptor | 1.678071905 | DOXL2 | diamine oxidase-like | −2.472487771 |
| | 971(Olfr971) | | | protein 2(Dox12) | |
| OLFR374 | olfactory receptor | 1.678071905 | PKD1L3 | polycystin 1 like 3, transient | −2.472487771 |
| | 374(Olfr374) | | | receptor potential channel | |
| | | | | interacting | |
| NOS1AP | nitric oxide synthase 1 | 1.678071905 | ZC3H11A | zinc finger CCCH-type | −2.472487771 |
| | adaptor protein | | | containing 11A | |
| GALM | galactose mutarotase | 1.678071905 | LY6K | lymphocyte antigen 6 | −2.472487771 |
| | | | | complex, locus K | |
| MEGF9 | multiple EGF like | 1.678071905 | KLF7 | Kruppel like factor 7 | −2.474755307 |
| | domains 9 | | | | |
| CCDC66 | coiled-coil domain | 1.678071905 | BTLA | B and T lymphocyte | −2.475604026 |
| | containing 66 | | | associated | |
| LRRC40 | leucine rich repeat | 1.678071905 | CDON | cell adhesion associated, | −2.485426827 |
| | containing 40 | | | oncogene regulated | |
| RALA | RALA Ras like proto- | 1.678071905 | DDC | dopa decarboxylase | −2.485426827 |
| | oncogene A | | | | |
| YIPF4 | Yip1 domain family | 1.678071905 | GTF2A2 | general transcription factor | −2.485426827 |
| | member 4 | | | IIA subunit 2 | |
| TAL2 | T-cell acute lymphocytic | 1.678071905 | DTX4 | deltex E3 ubiquitin ligase 4 | −2.485426827 |
| | leukemia 2 | | | | |
| LRRC8A | leucine rich repeat | 1.678071905 | GSTK1 | glutathione S-transferase | −2.486195934 |
| | containing 8 family | | | kappa 1 | |
| | member A | | | | |
| APOM | apolipoprotein M | 1.678071905 | OLFR213 | olfactory receptor | −2.489125048 |
| | | | | 213(Olfr213) | |
| KCNG3 | potassium voltage-gated | 1.678071905 | PDE5A | phosphodiesterase 5A | −2.490571469 |
| | channel modifier | | | | |
| | subfamily G member 3 | | | | |
| CNN1 | calponin 1 | 1.678071905 | TOB1 | transducer of ERBB2, 1 | −2.496763907 |
| STAC2 | SH3 and cysteine rich | 1.678071905 | 1700109H08RIK | Description Not Found | −2.498250868 |
| | domain 2 | | | | |
| SFRP2 | secreted frizzled related | 1.678071905 | LEFTY1 | left-right determination | −2.498250868 |
| | protein 2 | | | factor 1 | |
| SERPINB9E | serine (or cysteine) | 1.670169131 | SNAPC4 | small nuclear RNA | −2.500878922 |
| | peptidase inhibitor, clade | | | activating complex | |
| | B, member 9e(Serpinb9e) | | | polypeptide 4 | |
| TFB1M | transcription factor B1, | 1.668946692 | RNF41 | ring finger protein 41 | −2.503551585 |
| | mitochondrial | | | | |
| SLC25A10 | solute carrier family 25 | 1.668856925 | KLHL34 | kelch like family member | −2.504620392 |
| | member 10 | | | 34 | |
| BID | BH3 interacting domain | 1.667992567 | SSH2 | slingshot protein | −2.505492762 |
| | death agonist | | | phosphatase 2 | |
| MRPS27 | mitochondrial ribosomal | 1.667295766 | CAMK2B | calcium/calmodulin | −2.507047355 |
| | protein S27 | | | dependent protein kinase II | |
| | | | | beta | |
| NEDD4 | neural precursor cell | 1.666756592 | IRF7 | interferon regulatory factor | −2.507590939 |
| | expressed, | | | 7 | |
| | developmentally down- | | | | |
| | regulated 4, E3 ubiquitin | | | | |
| | protein ligase | | | | |
| VANGL2 | VANGL planar cell | 1.666756592 | SCML4 | sex comb on midleg-like 4 | −2.523118672 |
| | polarity protein 2 | | | (Drosophila) | |
| UBE2R2 | ubiquitin conjugating | 1.666641116 | EPB4.1 | Description Not Found | −2.523561956 |
| | enzyme E2 R2 | | | | |
| KLHL30 | kelch like family member | 1.666519523 | PARP12 | poly(ADP-ribose) | −2.523561956 |
| | 30 | | | polymerase family member | |
| | | | | 12 | |
| FBXO36 | F-box protein 36 | 1.665588375 | CACNB3 | calcium voltage-gated | −2.529877218 |
| | | | | channel auxiliary subunit | |
| | | | | beta 3 | |
| DCT | dopachrome tautomerase | 1.664016818 | NRG4 | neuregulin 4 | −2.53318567 |
| CCDC120 | coiled-coil domain | 1.663931727 | OLFR1383 | olfactory receptor | −2.5360529 |
| | containing 120 | | | 1383(Olfr1383) | |
| TMEM38B | transmembrane protein | 1.663455268 | PTGR1 | prostaglandin reductase 1 | −2.5360529 |
| | 38B | | | | |
| ENDOD1 | endonuclease domain | 1.663327923 | NFAM1 | NFAT activating protein | −2.5360529 |
| | containing 1 | | | with ITAM motif 1 | |
| PTPRD | protein tyrosine | 1.663215776 | ARL4C | ADP ribosylation factor like | −2.5360529 |
| | phosphatase, receptor | | | GTPase 4C | |
| | type D | | | | |
| ARL3 | ADP ribosylation factor | 1.661690196 | LACE1 | lactation elevated 1 | −2.5360529 |
| | like GTPase 3 | | | | |
| CDC37 | cell division cycle 37 | 1.661567827 | CDC14B | cell division cycle 14B | −2.545350645 |
| MKKS | McKusick-Kaufman | 1.66106548 | GUCA1A | guanylate cyclase activator | −2.548436625 |
| | syndrome | | | 1A | |
| CHN2 | chimerin 2 | 1.660998764 | KIF21B | kinesin family member 21B | −2.554588852 |
| CRTAP | cartilage associated | 1.659431912 | ARID3B | AT-rich interaction domain | −2.558087884 |
| | protein | | | 3B | |
| CXCR6 | C-X-C motif chemokine | 1.657515938 | HBA-A1 | hemoglobin alpha, adult | −2.560714954 |
| | receptor 6 | | | chain 1(Hba-a1) | |
| BUB1B | BUB1 mitotic checkpoint | 1.65691495 | CSF2RB2 | colony stimulating factor 2 | −2.560714954 |
| | serine/threonine kinase B | | | receptor, beta 2, low- | |
| | | | | affinity (granulocyte- | |
| | | | | macrophage)(Csf2rb2) | |
| B430306N03RIK | RIKEN cDNA | 1.655351829 | ATP6V1B1 | ATPase H+ transporting V1 | −2.560714954 |
| | B430306N03 | | | subunit B1 | |
| | gene(B430306N03Rik) | | | | |
| OLFR1262 | olfactory receptor | 1.655351829 | PCSK1N | proprotein convertase | −2.560714954 |
| | 1262(Olfr1262) | | | subtilisin/kexin type 1 | |
| | | | | inhibitor | |
| SLC38A5 | solute carrier family 38 | 1.655351829 | ZFP667 | zinc finger protein | −2.566670372 |
| | member 5 | | | 667(Zfp667) | |
| VAT1L | vesicle amine transport 1- | 1.655351829 | SH3BP1 | SH3 domain binding | −2.566734604 |
| | like | | | protein 1 | |
| HOXB7 | homeobox B7 | 1.655351829 | FFAR2 | free fatty acid receptor 2 | −2.572889668 |
| GAN | gigaxonin | 1.655351829 | EEF2K | eukaryotic elongation factor | −2.572889668 |
| | | | | 2 kinase | |
| MMP28 | matrix metallopeptidase | 1.655351829 | SLPI | secretory leukocyte | −2.574721828 |
| | 28 | | | peptidase inhibitor | |
| METTL10 | methyltransferase like 10 | 1.655351829 | CMA1 | chymase 1 | −2.584962501 |
| SIX4 | SIX homeobox 4 | 1.655351829 | ASCL1 | achaete-scute family bHLH | −2.584962501 |
| | | | | transcription factor 1 | |
| TDRD6 | tudor domain containing 6 | 1.655351829 | ACPP | acid phosphatase, prostate | −2.584962501 |
| COMMD5 | COMM domain | 1.654604999 | CLCNKB | chloride voltage-gated | −2.596935142 |
| | containing 5 | | | channel Kb | |
| PRDX4 | peroxiredoxin 4 | 1.651923925 | FBXW7 | F-box and WD repeat | −2.596935142 |
| | | | | domain containing 7 | |
| HS3ST3A1 | heparan sulfate- | 1.649298274 | OLIG3 | oligodendrocyte | −2.596935142 |
| | glucosamine 3- | | | transcription factor 3 | |
| | sulfotransferase 3A1 | | | | |
| CALCA | calcitonin related | 1.649067786 | WHRN | whirlin | −2.606789951 |
| | polypeptide alpha | | | | |
| SLC12A2 | solute carrier family 12 | 1.648449243 | DNAJC14 | DnaJ heat shock protein | −2.608809243 |
| | member 2 | | | family (Hsp40) member | |
| | | | | C14 | |
| TJP2 | tight junction protein 2 | 1.644145647 | PIGT | phosphatidylinositol glycan | −2.611031218 |
| | | | | anchor biosynthesis class T | |
| LRRC16B | Description Not Found | 1.64385619 | AP1G2 | adaptor related protein | −2.614709844 |
| | | | | complex 1 gamma 2 subunit | |
| AP3S2 | adaptor related protein | 1.64385619 | SAA2 | serum amyloid A2 | −2.62058641 |
| | complex 3 sigma 2 | | | | |
| | subunit | | | | |
| PSMD9 | proteasome 26S subunit, | 1.64385619 | USP30 | ubiquitin specific peptidase | −2.62058641 |
| | non-ATPase 9 | | | 30 | |
| PARD6G | par-6 family cell polarity | 1.643379419 | RPE65 | retinal pigment epithelium | −2.632268216 |
| | regulator gamma | | | specific protein 65 | |
| CIAPIN1 | cytokine induced | 1.643219709 | CML1 | Description Not Found | −2.634891632 |
| | apoptosis inhibitor 1 | | | | |
| CKAP5 | cytoskeleton associated | 1.642747156 | SLC6A19 | solute carrier family 6 | −2.640930751 |
| | protein 5 | | | member 19 | |
| E430025E21RIK | RIKEN cDNA | 1.641902626 | FGF15 | fibroblast growth factor | −2.64385619 |
| | E430025E21 | | | 15(Fgf15) | |
| | gene(E430025E21Rik) | | | | |
| PIAS3 | protein inhibitor of | 1.641884484 | HERC3 | HECT and RLD domain | −2.64385619 |
| | activated STAT 3 | | | containing E3 ubiquitin | |
| | | | | protein ligase 3 | |
| USP1 | ubiquitin specific | 1.640233791 | ADAMTSL4 | ADAMTS like 4 | −2.64385619 |
| | peptidase 1 | | | | |
| RAB3GAP2 | RAB3 GTPase activating | 1.639592623 | HYAL3 | hyaluronoglucosaminidase | −2.64385619 |
| | non-catalytic protein | | | 3 | |
| | subunit 2 | | | | |
| CSRP2 | cysteine and glycine rich | 1.639046229 | SLC15A2 | solute carrier family 15 | −2.648217996 |
| | protein 2 | | | member 2 | |
| MOV10 | Mov10 RISC complex | 1.638073837 | UFSP1 | UFM1-specific peptidase 1 | −2.649553823 |
| | RNA helicase | | | (inactive) | |
| GM1965 | predicted gene | 1.637881562 | 6430573F11RIK | Description Not Found | −2.655351829 |
| | 1965(Gm1965) | | | | |
| POMGNT1 | protein O-linked mannose | 1.636237884 | DNM3OS | DNM3 opposite | −2.655351829 |
| | N- | | | strand/antisense RNA | |
| | acetylglucosaminyltransfe | | | | |
| | rase 1 (beta 1,2-) | | | | |
| FIGNL1 | fidgetin like 1 | 1.633950492 | F2RL1 | F2R like trypsin receptor 1 | −2.655351829 |
| TMEM177 | transmembrane protein | 1.633475547 | SNX33 | sorting nexin 33 | −2.666654581 |
| | 177 | | | | |
| ALX4 | ALX homeobox 4 | 1.632864872 | CXCL9 | C-X-C motif chemokine | −2.666756592 |
| | | | | ligand 9 | |
| OLFR533 | olfactory receptor | 1.632268216 | TEAD2 | TEA domain transcription | −2.666756592 |
| | 533(Olfr533) | | | factor 2 | |
| H2-M10.3 | histocompatibility 2, M | 1.632268216 | QSOX1 | quiescin sulfhydryl oxidase | −2.666756592 |
| | region locus 10.3(H2- | | | 1 | |
| | M10.3) | | | | |
| GPX7 | glutathione peroxidase 7 | 1.632268216 | TLR13 | toll-like receptor 13(Tlr13) | −2.678071905 |
| STXBP6 | syntaxin binding protein 6 | 1.632268216 | SCD3 | stearoyl-coenzyme A | −2.678071905 |
| | | | | desaturase 3(Scd3) | |
| RAB33A | RAB33A, member RAS | 1.632268216 | SDC3 | syndecan 3 | −2.678071905 |
| | oncogene family | | | | |
| PDCL3 | phosducin like 3 | 1.632268216 | GRPR | gastrin releasing peptide | −2.678071905 |
| | | | | receptor | |
| GPR20 | G protein-coupled | 1.632268216 | MAFK | MAF bZIP transcription | −2.678071905 |
| | receptor 20 | | | factor K | |
| GSTA2 | glutathione S-transferase | 1.632268216 | DIRC2 | disrupted in renal | −2.678071905 |
| | alpha 2 | | | carcinoma 2 | |
| ADCY10 | adenylate cyclase 10 | 1.632268216 | ZCCHC12 | zinc finger CCHC-type | −2.67833354 |
| | (soluble) | | | containing 12 | |
| PEX12 | peroxisomal biogenesis | 1.632268216 | ADCY6 | adenylate cyclase 6 | −2.680886921 |
| | factor 12 | | | | |
| IQCC | IQ motif containing C | 1.632268216 | ECM1 | extracellular matrix protein | −2.68345512 |
| | | | | 1 | |
| ENPP1 | ectonucleotide | 1.632086279 | AFP | alpha fetoprotein | −2.689299161 |
| | pyrophosphatase/ | | | | |
| | phosphodiesterase 1 | | | | |
| ADAL | adenosine deaminase-like | 1.630664126 | GP5 | glycoprotein V platelet | −2.689299161 |
| SCRN2 | secernin 2 | 1.630566247 | GAB3 | GRB2 associated binding | −2.691405681 |
| | | | | protein 3 | |
| CEP78 | centrosomal protein 78 | 1.629851642 | USP2 | ubiquitin specific peptidase | −2.693334369 |
| | | | | 2 | |
| SLC25A15 | solute carrier family 25 | 1.629798606 | PLXNB1 | plexin B1 | −2.700439718 |
| | member 15 | | | | |
| ADSSL1 | adenylosuccinate synthase | 1.628272149 | PODXL2 | podocalyxin like 2 | −2.700799925 |
| | like 1 | | | | |
| TM6SF2 | transmembrane 6 | 1.627758638 | RAD9B | RAD9 checkpoint clamp | −2.70103836 |
| | superfamily member 2 | | | component B | |
| TUBG1 | tubulin gamma 1 | 1.624511879 | AKAP10 | A-kinase anchoring protein | −2.705977902 |
| | | | | 10 | |
| FASTK | Fas activated | 1.623336662 | PIGW | phosphatidylinositol glycan | −2.716990894 |
| | serine/threonine kinase | | | anchor biosynthesis class W | |
| RBBP5 | RB binding protein 5, | 1.622163711 | COL12A1 | collagen type XII alpha 1 | −2.722466024 |
| | histone lysine | | | chain | |
| | methyltransferase | | | | |
| | complex subunit | | | | |
| 1700071K01RIK | Description Not Found | 1.621465074 | GPR137B | G protein-coupled receptor | −2.733354341 |
| | | | | 137B | |
| SLC25A33 | solute carrier family 25 | 1.621282718 | IMMP2L | inner mitochondrial | −2.733354341 |
| | member 33 | | | membrane peptidase | |
| | | | | subunit 2 | |
| MDM4 | MDM4, p53 regulator | 1.62058641 | PIK3CB | phosphatidylinositol-4,5- | −2.737320423 |
| | | | | bisphosphate 3-kinase | |
| | | | | catalytic subunit beta | |
| TOP2A | topoisomerase (DNA) II | 1.620374948 | TGFBI | transforming growth factor | −2.740276443 |
| | alpha | | | beta induced | |
| OLFR139 | olfactory receptor | 1.619731323 | ZFP106 | zinc finger protein | −2.744161096 |
| | 139(Olfr139) | | | 106(Zfp106) | |
| PAPLN | papilin, proteoglycan like | 1.618762248 | ARNTL | aryl hydrocarbon receptor | −2.744161096 |
| | sulfated glycoprotein | | | nuclear translocator like | |
| PACSIN2 | protein kinase C and | 1.617401771 | HS3ST3B1 | heparan sulfate- | −2.744161096 |
| | casein kinase substrate in | | | glucosamine 3- | |
| | neurons 2 | | | sulfotransferase 3B1 | |
| TRDMT1 | tRNA aspartic acid | 1.615239219 | OASL2 | 2′-5′ oligoadenylate | −2.744892108 |
| | methyltransferase 1 | | | synthetase-like 2(Oas12) | |
| 4932438H23RIK | Description Not Found | 1.614681809 | PRDX6 | peroxiredoxin 6 | −2.75084462 |
| SPAG9 | sperm associated antigen | 1.614567709 | RASA2 | RAS p21 protein activator 2 | −2.751203108 |
| | 9 | | | | |
| RPA3 | replication protein A3 | 1.61436984 | HOXB2 | homeobox B2 | −2.754887502 |
| GNPTAB | N-acetylglucosamine-1- | 1.613298199 | TULP3 | tubby like protein 3 | −2.754887502 |
| | phosphate transferase | | | | |
| | alpha and beta subunits | | | | |
| SNX9 | sorting nexin 9 | 1.609251493 | MFRP | membrane frizzled-related | −2.754887502 |
| | | | | protein | |
| OLFR550 | olfactory receptor | 1.609195813 | MEN1 | menin 1 | −2.757556689 |
| | 550(Olfr550) | | | | |
| ZFP160 | zinc finger protein | 1.608809243 | C330021F23RIK | RIKEN cDNA | −2.762199201 |
| | 160(Zfp160) | | | C330021F23 | |
| | | | | gene(C330021F23Rik) | |
| TAS2R129 | taste receptor, type 2, | 1.608809243 | CSTAD | CSA-conditional, T cell | −2.765534746 |
| | member 129(Tas2r129) | | | activation-dependent | |
| | | | | protein(Cstad) | |
| OLFR371 | olfactory receptor | 1.608809243 | ALDH5A1 | aldehyde dehydrogenase 5 | −2.773022439 |
| | 371(Olfr371) | | | family member A1 | |
| OLFR281 | olfactory receptor | 1.608809243 | EPM2AIP1 | EPM2A interacting protein | −2.773468928 |
| | 281(Olfr281) | | | 1 | |
| OLFR195 | olfactory receptor | 1.608809243 | PDE8B | phosphodiesterase 8B | −2.776103988 |
| | 195(Olfr195) | | | | |
| OLFR142 | olfactory receptor | 1.608809243 | DMRTA1 | DMRT like family A1 | −2.776184379 |
| | 142(Olfr142) | | | | |
| PRSS3 | protease, serine 3 | 1.608809243 | LYPD6B | LY6/PLAUR domain | −2.780048768 |
| | | | | containing 6B | |
| CX3CL1 | C-X3-C motif chemokine | 1.608809243 | CD300E | CD300e molecule | −2.786596362 |
| | ligand 1 | | | | |
| TMPRSS6 | transmembrane protease, | 1.608809243 | NPFF | neuropeptide FF-amide | −2.786596362 |
| | serine 6 | | | peptide precursor | |
| ALK | anaplastic lymphoma | 1.608809243 | FASTKD1 | FAST kinase domains 1 | −2.793765229 |
| | receptor tyrosine kinase | | | | |
| ITGA9 | integrin subunit alpha 9 | 1.608809243 | OLFR802 | olfactory receptor | −2.797012978 |
| | | | | 802(Olfr802) | |
| TIMM13 | translocase of inner | 1.608809243 | HIVEP1 | human immunodeficiency | −2.797012978 |
| | mitochondrial membrane | | | virus type I enhancer | |
| | 13 | | | binding protein 1 | |
| MSH5 | mutS homolog 5 | 1.608809243 | HIC1 | hypermethylated in cancer 1 | −2.797012978 |
| XPO4 | exportin 4 | 1.605818241 | TRIM33 | tripartite motif containing | −2.802009226 |
| | | | | 33 | |
| MED21 | mediator complex subunit | 1.603309406 | SELL | selectin L | −2.803274253 |
| CHST12 | carbohydrate | 1.602612589 | EPHX1 | epoxide hydrolase 1 | −2.803758579 |
| | sulfotransferase 12 | | | | |
| 6030408B16RIK | Description Not Found | 1.602195565 | BCL9 | B-cell CLL/lymphoma 9 | −2.807354922 |
| SLU7 | SLU7 homolog, splicing | 1.601548066 | STAT2 | signal transducer and | −2.808521822 |
| | factor | | | activator of transcription 2 | |
| CDK5RAP2 | CDK5 regulatory subunit | 1.601120229 | ELMO3 | engulfment and cell motility | −2.812498225 |
| | associated protein 2 | | | 3 | |
| CASP7 | caspase 7 | 1.6002402 | HDC | histidine decarboxylase | −2.815167456 |
| KIF22 | kinesin family member 22 | 1.599011705 | AI317395 | Description Not Found | −2.817623258 |
| E2F1 | E2F transcription factor 1 | 1.598449678 | RPL14 | ribosomal protein L14 | −2.817623258 |
| MXI1 | MAX interactor 1, | 1.597690116 | SNAI1 | snail family transcriptional | −2.818256244 |
| | dimerization protein | | | repressor 1 | |
| DONSON | downstream neighbor of | 1.596935142 | NUPR1 | nuclear protein 1, | −2.827819025 |
| | SON | | | transcriptional regulator | |
| TBX22 | T-box 22 | 1.596935142 | IGSF8 | immunoglobulin | −2.827819025 |
| | | | | superfamily member 8 | |
| INPPL1 | inositol polyphosphate | 1.596300192 | SLC12A7 | solute carrier family 12 | −2.827819025 |
| | phosphatase like 1 | | | member 7 | |
| CSE1L | chromosome segregation | 1.59586273 | RENBP | renin binding protein | −2.837431463 |
| | 1 like | | | | |
| NDFIP2 | Nedd4 family interacting | 1.594709608 | ZFP553 | zinc finger protein | −2.837943242 |
| | protein 2 | | | 553(Zfp553) | |
| LYPD6 | LY6/PLAUR domain | 1.592962293 | LRFN2 | leucine rich repeat and | −2.837943242 |
| | containing 6 | | | fibronectin type III domain | |
| | | | | containing 2 | |
| DDX49 | DEAD-box helicase 49 | 1.592190323 | HP | haptoglobin | −2.839737506 |
| MGLL | monoglyceride lipase | 1.590948822 | TOMM40 | translocase of outer | −2.847996907 |
| | | | | mitochondrial membrane 40 | |
| NR4A3 | nuclear receptor | 1.59092994 | GABARAPL2 | GABA type A receptor | −2.847996907 |
| | subfamily 4 group A | | | associated protein like 2 | |
| | member 3 | | | | |
| LRRN3 | leucine rich repeat | 1.590360181 | TMEM86A | transmembrane protein 86A | −2.855497819 |
| | neuronal 3 | | | | |
| PTPRK | protein tyrosine | 1.587927102 | LRP1 | LDL receptor related | −2.857980995 |
| | phosphatase, receptor | | | protein 1 | |
| | type K | | | | |
| OLFR1212 | olfactory receptor | 1.584962501 | ATXN1 | ataxin 1 | −2.857980995 |
| | 1212(Olfr1212) | | | | |
| KLHL2 | kelch like family member | 1.584962501 | FAS | Fas cell surface death | −2.861524641 |
| | 2 | | | receptor | |
| UBE2G2 | ubiquitin conjugating | 1.584962501 | ZDHHC18 | zinc finger DHHC-type | −2.882740655 |
| | enzyme E2 G2 | | | containing 18 | |
| GRIN2A | glutamate ionotropic | 1.584962501 | LARGE | Description Not Found | −2.887525271 |
| | receptor NMDA type | | | | |
| | subunit 2A | | | | |
| INHA | inhibin alpha subunit | 1.584962501 | SP5 | Sp5 transcription factor | −2.887525271 |
| RNPC3 | RNA binding region | 1.584962501 | ATG7 | autophagy related 7 | −2.895440528 |
| | (RNP1, RRM) containing | | | | |
| | 3 | | | | |
| XKR7 | XK related 7 | 1.584962501 | DNAJC27 | DnaJ heat shock protein | −2.897240426 |
| | | | | family (Hsp40) member | |
| | | | | C27 | |
| STX19 | syntaxin 19 | 1.584962501 | PCSK4 | proprotein convertase | −2.900866808 |
| | | | | subtilisin/kexin type 4 | |
| SLC5A5 | solute carrier family 5 | 1.584962501 | RNF141 | ring finger protein 141 | −2.902073579 |
| | member 5 | | | | |
| VPS37C | VPS37C, ESCRT-I | 1.584022655 | GRAP2 | GRB2-related adaptor | −2.904150467 |
| | subunit | | | protein 2 | |
| ERMP1 | endoplasmic reticulum | 1.582531434 | VIPR1 | vasoactive intestinal peptide | −2.904484098 |
| | metallopeptidase 1 | | | receptor 1 | |
| ZFP790 | zinc finger protein | 1.581046002 | CAR15 | carbonic anhydrase | −2.906890596 |
| | 790(Zfp790) | | | 15(Car15) | |
| AA467197 | expressed sequence | 1.579947242 | RELL2 | RELT like 2 | −2.906890596 |
| | AA467197(AA467197) | | | | |
| UBE2Z | ubiquitin conjugating | 1.57976541 | HECA | hdc homolog, cell cycle | −2.916545968 |
| | enzyme E2 Z | | | regulator | |
| SOAT2 | sterol O-acyltransferase 2 | 1.577460518 | DPM1 | dolichyl-phosphate | −2.933100475 |
| | | | | mannosyltransferase | |
| | | | | polypeptide 1, catalytic | |
| | | | | subunit | |
| ZMAT5 | zinc finger matrin-type 5 | 1.576986214 | AOC2 | amine oxidase, copper | −2.936235748 |
| | | | | containing 2 | |
| CDCA3 | cell division cycle | 1.576323153 | HIST2H2BE | histone cluster 2, H2be | −2.936320631 |
| | associated 3 | | | | |
| NEUROD2 | neuronal differentiation 2 | 1.576266476 | ACAD10 | acyl-CoA dehydrogenase | −2.942514505 |
| | | | | family member 10 | |
| WDR35 | WD repeat domain 35 | 1.576120636 | NT5E | 5′-nucleotidase ecto | −2.944039663 |
| TWSG1 | twisted gastrulation BMP | 1.5758728 | SSH1 | slingshot protein | −2.944858446 |
| | signaling modulator 1 | | | phosphatase 1 | |
| PPT1 | palmitoyl-protein | 1.575321868 | SEMA4F | ssemaphorin 4F | −2.948329995 |
| | thioesterase 1 | | | | |
| IRF8 | interferon regulatory | 1.574489283 | NKD2 | naked cuticle homolog 2 | −2.953960396 |
| | factor 8 | | | | |
| PLEKHG5 | pleckstrin homology and | 1.574066379 | TCEB3 | transcription elongation | −2.95419631 |
| | RhoGEF domain | | | factor B subunit 3 | |
| | containing G5 | | | | |
| CDC20 | cell division cycle 20 | 1.573718243 | HDAC4 | histone deacetylase 4 | −2.95419631 |
| MFI2 | antigen p97 (melanoma | 1.572889668 | PCNX | pecanex homolog | −2.972692654 |
| | associated) identified by | | | (Drosophila)(Pcnx) | |
| | monoclonal antibodies | | | | |
| | 133.2 and 96.5(Mfi2) | | | | |
| HDAC9 | histone deacetylase 9 | 1.571625208 | ARL5C | ADP ribosylation factor like | −2.972692654 |
| | | | | GTPase 5C | |
| ASF1B | anti-silencing function 1B | 1.570544039 | 1600014C10RIK | Description Not Found | −2.981852653 |
| | histone chaperone | | | | |
| B3GNT1 | Description Not Found | 1.569171715 | ANKRD23 | ankyrin repeat domain 23 | −2.981852653 |
| SLC25A14 | solute carrier family 25 | 1.569127395 | CLOCK | clock circadian regulator | −2.985543793 |
| | member 14 | | | | |
| FYN | FYN proto-oncogene, Src | 1.567462919 | SFI1 | SFI1 centrin binding protein | −2.986410935 |
| | family tyrosine kinase | | | | |
| SERPINB6B | serine (or cysteine) | 1.567348435 | HEY1 | hes related family bHLH | −2.987632559 |
| | peptidase inhibitor, clade | | | transcription factor with | |
| | B, member 6b(Serpinb6b) | | | YRPW motif 1 | |
| TOP1MT | topoisomerase (DNA) I, | 1.567180597 | ATP11C | ATPase phospholipid | −2.99095486 |
| | mitochondrial | | | transporting 11C | |
| CCDC50 | coiled-coil domain | 1.566273906 | NUDCD3 | NudC domain containing 3 | −3 |
| | containing 50 | | | | |
| ZFP414 | zinc finger protein | 1.565776574 | CDC25A | cell division cycle 25A | −3.000238201 |
| | 414(Zfp414) | | | | |
| OGFOD2 | 2-oxoglutarate and iron | 1.565512016 | OLFR135 | olfactory receptor | −3.017921908 |
| | dependent oxygenase | | | 135(Olfr135) | |
| | domain containing 2 | | | | |
| CTNNAL1 | catenin alpha like 1 | 1.563586461 | RC3H1 | ring finger and CCCH-type | −3.019621529 |
| | | | | domains 1 | |
| CREB3L2 | CAMP responsive element | 1.561361122 | NSG2 | neuron specific gene family | −3.020466888 |
| | binding protein 3 like 2 | | | member 2(Nsg2) | |
| OLFR492 | olfactory receptor | 1.560714954 | ID1 | inhibitor of DNA binding 1, | −3.026800059 |
| | 492(Olfr492) | | | HLH protein | |
| OLFR1312 | olfactory receptor | 1.560714954 | CYP2D22 | cytochrome P450, family 2, | −3.044282215 |
| | 1312(Olfr1312) | | | subfamily d, polypeptide | |
| | | | | 22(Cyp2d22) | |
| UPK2 | uroplakin 2 | 1.560714954 | H2AFJ | H2A histone family | −3.044297135 |
| | | | | member J | |
| RESP18 | regulated endocrine | 1.560714954 | TGFBR3 | transforming growth factor | −3.053111336 |
| | specific protein 18 | | | beta receptor 3 | |
| CRCT1 | cysteine rich C-terminal 1 | 1.560714954 | IRS2 | insulin receptor substrate 2 | −3.061776198 |
| NEUROD4 | neuronal differentiation 4 | 1.560714954 | ADCY7 | adenylate cyclase 7 | −3.06608919 |
| SENP1 | SUMO1/sentrin specific | 1.560714954 | HYI | hydroxypyruvate isomerase | −3.072315809 |
| | peptidase 1 | | | (putative) | |
| MR1 | major histocompatibility | 1.560714954 | TRIP4 | thyroid hormone receptor | −3.078951341 |
| | complex, class I-related | | | interactor 4 | |
| BIVM | basic, immunoglobulin- | 1.560714954 | D730001G18RIK | RIKEN cDNA | −3.087462841 |
| | like variable motif | | | D730001G18 | |
| | containing | | | gene(D730001G18Rik) | |
| KPNA2 | karyopherin subunit alpha | 1.560714954 | PRR7 | proline rich 7 (synaptic) | −3.087462841 |
| | 2 | | | | |
| BAG2 | BCL2 associated | 1.560714954 | GFPT2 | glutamine-fructose-6- | −3.09592442 |
| | athanogene 2 | | | phosphate transaminase 2 | |
| SLC12A8 | solute carrier family 12 | 1.560714954 | SCMH1 | sex comb on midleg | −3.100136671 |
| | member 8 | | | homolog 1 (Drosophila) | |
| SCN7A | sodium voltage-gated | 1.560714954 | ANKRD12 | ankyrin repeat domain 12 | −3.107456458 |
| | channel alpha subunit 7 | | | | |
| SLC5A7 | solute carrier family 5 | 1.560714954 | PTPRV | protein tyrosine | −3.112700133 |
| | member 7 | | | phosphatase, receptor type, | |
| | | | | V(Ptprv) | |
| ENPEP | glutamyl aminopeptidase | 1.560714954 | TMEM135 | transmembrane protein 135 | −3.112700133 |
| ANGPTL4 | angiopoietin like 4 | 1.56060777 | AKAP3 | A-kinase anchoring protein | −3.11460665 |
| | | | | 3 | |
| OSBPL3 | oxysterol binding protein | 1.559778376 | CBR2 | carbonyl reductase 2(Cbr2) | −3.129283017 |
| | like 3 | | | | |
| MCFD2 | multiple coagulation | 1.559617874 | CXCL16 | C-X-C motif chemokine | −3.129283017 |
| | factor deficiency 2 | | | ligand 16 | |
| MAP2K1 | mitogen-activated protein | 1.558556708 | MBTD1 | mbt domain containing 1 | −3.145677455 |
| | kinase kinase 1 | | | | |
| ING2 | inhibitor of growth family | 1.557223521 | UBE2J2 | ubiquitin conjugating | −3.161887682 |
| | member 2 | | | enzyme E2 J2 | |
| CDCA5 | cell division cycle | 1.55643411 | STK36 | serine/threonine kinase 36 | −3.161887682 |
| | associated 5 | | | | |
| MAP3K7 | mitogen-activated protein | 1.554463905 | SLC14A1 | solute carrier family 14 | −3.16922072 |
| | kinase kinase kinase 7 | | | member 1 (Kidd blood | |
| | | | | group) | |
| GSTT3 | glutathione S-transferase, | 1.55048277 | CTSE | cathepsin E | −3.177917792 |
| | theta 3(Gstt3) | | | | |
| PFN2 | profilin 2 | 1.549690793 | HSD3B7 | hydroxy-delta-5-steroid | −3.177917792 |
| | | | | dehydrogenase, 3 beta- and | |
| | | | | steroid delta-isomerase 7 | |
| HPS4 | HPS4, biogenesis of | 1.549115647 | 3010003L21RIK | Description Not Found | −3.179249632 |
| | lysosomal organelles | | | | |
| | complex 3 subunit 2 | | | | |
| CAPN8 | calpain 8 | 1.548436625 | BAI1 | Description Not Found | −3.186461055 |
| RAB11FIP5 | RAB11 family interacting | 1.548436625 | ZFP451 | zinc finger protein | −3.187711618 |
| | protein 5 | | | 451(Zfp451) | |
| CD9 | CD9 molecule | 1.548429184 | CCDC28B | coiled-coil domain | −3.192207249 |
| | | | | containing 28B | |
| CCR6 | C-C motif chemokine | 1.548250633 | MCF2L | MCF.2 cell line derived | −3.199672345 |
| | receptor 6 | | | transforming sequence like | |
| ALG2 | ALG2, alpha-1,3/1,6- | 1.547992668 | BCL6 | B-cell CLL/lymphoma 6 | −3.201024389 |
| | mannosyltransferase | | | | |
| BCDIN3D | BCDIN3 domain | 1.546046129 | PFKFB4 | 6-phosphofructo-2- | −3.204935584 |
| | containing RNA | | | kinase/fructose-2,6- | |
| | methyltransferase | | | biphosphatase 4 | |
| NT5DC3 | 5′-nucleotidase domain | 1.54522349 | PROS1 | protein S (alpha) | −3.209453366 |
| | containing 3 | | | | |
| DNAJC18 | DnaJ heat shock protein | 1.544626916 | CTSH | cathepsin H | −3.21628737 |
| | family (Hsp40) member | | | | |
| | C18 | | | | |
| SH3RF1 | SH3 domain containing | 1.544156019 | CRTC3 | CREB regulated | −3.217230716 |
| | ring finger 1 | | | transcription coactivator 3 | |
| RGS16 | regulator of G-protein | 1.541382294 | TNKS | tankyrase | −3.217230716 |
| | signaling 16 | | | | |
| NCAPH | non-SMC condensin I | 1.540788228 | GRM6 | glutamate metabotropic | −3.224966365 |
| | complex subunit H | | | receptor 6 | |
| USP14 | ubiquitin specific | 1.540333713 | SPSB1 | splA/ryanodine receptor | −3.255500733 |
| | peptidase 14 | | | domain and SOCS box | |
| | | | | containing 1 | |
| RFT1 | RFT1 homolog | 1.54031759 | PARP8 | poly(ADP-ribose) | −3.263034406 |
| | | | | polymerase family member | |
| | | | | 8 | |
| SLC31A1 | solute carrier family 31 | 1.540275536 | KCNRG | potassium channel regulator | −3.263034406 |
| | member 1 | | | | |
| TCTEX1D2 | Tctex1 domain containing | 1.538332378 | POU6F1 | POU class 6 homeobox 1 | −3.268517714 |
| | 2 | | | | |
| TTF2 | transcription termination | 1.537871953 | REV3L | REV3 like, DNA directed | −3.270528942 |
| | factor 2 | | | polymerase zeta catalytic | |
| | | | | subunit | |
| ZFP7 | zinc finger protein | 1.5360529 | TCF7 | transcription factor 7 (T-cell | −3.272419178 |
| | 7(Zfp7) | | | specific, HMG-box) | |
| G6PD2 | glucose-6-phosphate | 1.5360529 | NME4 | NME/NM23 nucleoside | −3.283551423 |
| | dehydrogenase 2(G6pd2) | | | diphosphate kinase 4 | |
| DEFB14 | defensin beta 14(Defb14) | 1.5360529 | PLAUR | plasminogen activator, | −3.285402219 |
| | | | | urokinase receptor | |
| SLC18A3 | solute carrier family 18 | 1.5360529 | CD4 | CD4 molecule | −3.285402219 |
| | member A3 | | | | |
| AHNAK2 | AHNAK nucleoprotein 2 | 1.5360529 | ZMYND11 | zinc finger MYND-type | −3.293186363 |
| | | | | containing 11 | |
| HOXC12 | homeobox C12 | 1.5360529 | ARMCX5 | armadillo repeat containing, | −3.298404158 |
| | | | | X-linked 5 | |
| CEACAM16 | carcinoembryonic antigen | 1.5360529 | LPHN1 | Description Not Found | −3.300123725 |
| | related cell adhesion | | | | |
| | molecule 16 | | | | |
| MOSPD3 | motile sperm domain | 1.5360529 | PIK3IP1 | phosphoinositide-3-kinase | −3.307428525 |
| | containing 3 | | | interacting protein 1 | |
| DCTN1 | dynactin subunit 1 | 1.5360529 | ERDR1 | erythroid differentiation | −3.317651188 |
| | | | | regulator 1(Erdr1) | |
| MYB | MYB proto-oncogene, | 1.5360529 | PLD4 | phospholipase D family | −3.328444792 |
| | transcription factor | | | member 4 | |
| GLIPR1L2 | GLI pathogenesis-related | 1.5360529 | BMF | Bcl2 modifying factor | −3.336283388 |
| | 1 like 2 | | | | |
| ALDH1A3 | aldehyde dehydrogenase | 1.5360529 | GALNT11 | polypeptide N- | −3.345118795 |
| | 1 family member A3 | | | acetylgalactosaminyl- | |
| | | | | transferase 11 | |
| SLC2A8 | solute carrier family 2 | 1.5360529 | LCN2 | lipocalin 2 | −3.378511623 |
| | member 8 | | | | |
| SRC | SRC proto-oncogene, | 1.5360529 | PAG1 | phosphoprotein membrane | −3.385431037 |
| | non-receptor tyrosine | | | anchor with | |
| | kinase | | | glycosphingolipid | |
| | | | | microdomains 1 | |
| ZCCHC17 | zinc finger CCHC-type | 1.535618518 | DTX1 | deltex E3 ubiquitin ligase 1 | −3.425576064 |
| | containing 17 | | | | |
| HNRNPUL1 | heterogeneous nuclear | 1.534420207 | RFFL | ring finger and FYVE-like | −3.426684082 |
| | ribonucleoprotein U like 1 | | | domain containing E3 | |
| | | | | ubiquitin protein ligase | |
| TRIM68 | tripartite motif containing | 1.533057052 | MAFF | MAF bZIP transcription | −3.429615964 |
| | 68 | | | factor F | |
| TPST1 | tyrosylprotein | 1.53140111 | TOR1AIP2 | torsin 1A interacting protein | −3.432316325 |
| | sulfotransferase 1 | | | 2 | |
| OLFR922 | olfactory receptor | 1.531260941 | SNN | stannin | −3.432316325 |
| | 922(Olfr922) | | | | |
| FIG4 | FIG4 phosphoinositide 5- | 1.530442167 | CLEC4N | C-type lectin domain family | −3.433567144 |
| | phosphatase | | | 4, member n(Clec4n) | |
| SETMAR | SET domain and mariner | 1.530442167 | RREB1 | ras responsive element | −3.443780274 |
| | transposase fusion gene | | | binding protein 1 | |
| GSTM5 | glutathione S-transferase | 1.530053218 | CCDC84 | coiled-coil domain | −3.445188687 |
| | mu 5 | | | containing 84 | |
| TUBA3B | tubulin, alpha | 1.527986221 | ID3 | inhibitor of DNA binding 3, | −3.46350285 |
| | 3B(Tuba3b) | | | HLH protein | |
| PDCL | phosducin like | 1.527807072 | BC065397 | cDNA sequence | −3.465974465 |
| | | | | BC065397(BC065397) | |
| SMPDL3B | sphingomyelin | 1.527243888 | VRK1 | vaccinia related kinase 1 | −3.46760555 |
| | phosphodiesterase acid | | | | |
| | like 3B | | | | |
| ABHD14A | abhydrolase domain | 1.527213882 | HOXD13 | homeobox D13 | −3.491853096 |
| | containing 14A | | | | |
| TIPIN | TIMELESS interacting | 1.526972991 | MAPK8IP2 | mitogen-activated protein | −3.491853096 |
| | protein | | | kinase 8 interacting protein | |
| | | | | 2 | |
| DSCC1 | DNA replication and | 1.525986429 | HOXA5 | homeobox A5 | −3.517275693 |
| | sister chromatid cohesion | | | | |
| | 1 | | | | |
| PSMD1 | proteasome 26S subunit, | 1.525574957 | HIST1H1A | histone cluster 1, Hla | −3.523561956 |
| | non-ATPase 1 | | | | |
| BZRAP1 | benzodiazepine receptor | 1.524166255 | MAML1 | mastermind like | −3.523603553 |
| | associated protein | | | transcriptional coactivator 1 | |
| | 1(Bzrap1) | | | | |
| ENO3 | enolase 3 | 1.523778831 | PTPDC1 | protein tyrosine | −3.526694846 |
| | | | | phosphatase domain | |
| | | | | containing 1 | |
| E330034G19RIK | RIKEN cDNA | 1.523561956 | TNFRSF12A | tumor necrosis factor | −3.528725998 |
| | E330034G19 | | | receptor superfamily | |
| | gene(E330034G19Rik) | | | member 12A | |
| GABRP | gamma-aminobutyric acid | 1.523561956 | TNIP2 | TNFAIP3 interacting | −3.539158811 |
| | type A receptor pi subunit | | | protein 2 | |
| SLC14A2 | solute carrier family 14 | 1.523561956 | HIST2H4 | histone cluster 2, | −3.540773411 |
| | member 2 | | | H4(Hist2h4) | |
| YWHAE | tyrosine 3- | 1.522478712 | PIM2 | Pim-2 proto-oncogene, | −3.557655155 |
| | monooxygenase/tryptoph | | | serine/threonine kinase | |
| | an 5-monooxygenase | | | | |
| | activation protein epsilon | | | | |
| EHBP1L1 | EH domain binding | 1.522282169 | DOK7 | docking protein 7 | −3.567781854 |
| | protein 1 like 1 | | | | |
| CHGB | chromogranin B | 1.51924262 | TNFSF14 | tumor necrosis factor | −3.588895735 |
| | | | | superfamily member 14 | |
| TXNRD2 | thioredoxin reductase 2 | 1.519008256 | TDRKH | tudor and KH domain | −3.590961241 |
| | | | | containing | |
| NCF1 | neutrophil cytosolic factor | 1.518873761 | FIBCD1 | fibrinogen C domain | −3.608656121 |
| | 1 | | | containing 1 | |
| OAF | out at first homolog | 1.517431856 | RBBP9 | RB binding protein 9, serine | −3.608809243 |
| | | | | hydrolase | |
| FAM110A | family with sequence | 1.517263583 | DERL1 | derlin 1 | −3.617651119 |
| | similarity 110 member A | | | | |
| ANGEL1 | angel homolog 1 | 1.515832566 | LENG9 | leukocyte receptor cluster | −3.62058641 |
| | (Drosophila) | | | member 9 | |
| RTN4IP1 | reticulon 4 interacting | 1.515760776 | TRPC2 | transient receptor potential | −3.62058641 |
| | protein 1 | | | cation channel subfamily C | |
| | | | | member 2, pseudogene | |
| LAMP2 | lysosomal associated | 1.515709038 | CCDC134 | coiled-coil domain | −3.632268216 |
| | membrane protein 2 | | | containing 134 | |
| KRT4 | keratin 4 | 1.514299789 | OAS2 | 2′-5′-oligoadenylate | −3.632268216 |
| | | | | synthetase 2 | |
| PAFAH1B3 | platelet activating factor | 1.5142935 | 2410127L17RIK | Description Not Found | −3.646738698 |
| | acetylhydrolase 1b | | | | |
| | catalytic subunit 3 | | | | |
| STT3A | STT3A, catalytic subunit | 1.513537695 | RSAD1 | radical S-adenosyl | −3.649220471 |
| | of the | | | methionine domain | |
| | oligosaccharyltransferase | | | containing 1 | |
| | complex | | | | |
| PRKAR1B | protein kinase cAMP- | 1.51340003 | H2-DMB1 | histocompatibility 2, class | −3.649615459 |
| | dependent type I | | | II, locus Mb1(H2-DMb1) | |
| | regulatory subunit beta | | | | |
| HIST1H2BB | histone cluster 1, H2bb | 1.512941595 | IFT81 | intraflagellar transport 81 | −3.673839056 |
| ZFP39 | zinc finger protein | 1.511385424 | MID1 | midline 1 | −3.683696454 |
| | 39(Zfp39) | | | | |
| PLK1 | polo like kinase 1 | 1.511151166 | DEPDC1B | DEP domain containing 1B | −3.683696454 |
| 1700028P14RIK | Description Not Found | 1.510961919 | SMAD3 | SMAD family member 3 | −3.716296166 |
| D10BWG1379E | Description Not Found | 1.510961919 | UBTD1 | ubiquitin domain containing | −3.716990894 |
| | | | | 1 | |
| TREM3 | triggering receptor | 1.510961919 | FBXO44 | F-box protein 44 | −3.738767837 |
| | expressed on myeloid | | | | |
| | cells 3(Trem3) | | | | |
| GM128 | predicted gene | 1.510961919 | KCNMB4 | potassium calcium- | −3.741951111 |
| | 128(Gm128) | | | activated channel subfamily | |
| | | | | M regulatory beta subunit 4 | |
| OLFR741 | olfactory receptor | 1.510961919 | FAIM3 | Description Not Found | −3.754887502 |
| | 741(Olfr741) | | | | |
| OLFR523 | olfactory receptor | 1.510961919 | CCM2 | CCM2 scaffolding protein | −3.754887502 |
| | 523(Olfr523) | | | | |
| DCPP1 | demilune cell and parotid | 1.510961919 | DAG1 | dystroglycan 1 | −3.760220946 |
| | protein 1(Dcpp1) | | | | |
| RPRML | reprimo like | 1.510961919 | FCGR3 | Fc receptor, IgG, low | −3.776103988 |
| | | | | affinity III(Fcgr3) | |
| CHRD | chordin | 1.510961919 | ZNRF1 | zinc and ring finger 1, E3 | −3.776103988 |
| | | | | ubiquitin protein ligase | |
| C5AR1 | complement component | 1.510961919 | TLR1 | toll like receptor 1 | −3.786596362 |
| | 5a receptor 1 | | | | |
| APOA2 | apolipoprotein A2 | 1.510961919 | HSD17B11 | hydroxysteroid 17-beta | −3.789207575 |
| | | | | dehydrogenase 11 | |
| PRG2 | proteoglycan 2, pro | 1.510961919 | ZPBP | zona pellucida binding | −3.887525271 |
| | eosinophil major basic | | | protein | |
| | protein | | | | |
| VCAM1 | vascular cell adhesion | 1.510961919 | ZSWIM3 | zinc finger SWIM-type | −3.892391026 |
| | molecule 1 | | | containing 3 | |
| LY6G5B | lymphocyte antigen 6 | 1.510961919 | SOCS1 | suppressor of cytokine | −3.892391026 |
| | complex, locus G5B | | | signaling 1 | |
| AIM2 | absent in melanoma 2 | 1.510961919 | KLF9 | Kruppel like factor 9 | −3.902021342 |
| DMBX1 | diencephalon/ | 1.510961919 | AHSA2 | AHA1, activator of heat | −3.904760449 |
| | mesencephalon | | | shock 90 kDa protein | |
| | homeobox 1 | | | ATPase homolog 2 (yeast) | |
| HCN2 | hyperpolarization | 1.510961919 | DDHD1 | DDHD domain containing 1 | −3.914086097 |
| | activated cyclic | | | | |
| | nucleotide gated | | | | |
| | potassium channel 2 | | | | |
| MRGPRF | MAS related GPR family | 1.510961919 | CNKSR3 | CNKSR family member 3 | −3.930737338 |
| | member F | | | | |
| CYTH4 | cytohesin 4 | 1.510961919 | CPEB2 | cytoplasmic | −4.017516295 |
| | | | | polyadenylation element | |
| | | | | binding protein 2 | |
| ANGPTL3 | angiopoietin like 3 | 1.510961919 | TRP53BP2 | transformation related | −4.021932279 |
| | | | | protein 53 binding protein | |
| | | | | 2(Trp53bp2) | |
| DHX29 | DEAH-box helicase 29 | 1.510667738 | FAM178A | family with sequence | −4.03562391 |
| | | | | similarity 178, member | |
| | | | | A(Fam178a) | |
| PMPCB | peptidase, mitochondrial | 1.509477625 | RCN3 | reticulocalbin 3 | −4.03562391 |
| | processing beta subunit | | | | |
| HRH3 | histamine receptor H3 | 1.508554002 | SPTLC2 | serine palmitoyltransferase | −4.040015679 |
| | | | | long chain base subunit 2 | |
| ZFP282 | zinc finger protein | 1.507419453 | ZFP810 | zinc finger protein | −4.070389328 |
| | 282(Zfp282) | | | 810(Zfp810) | |
| TBC1D7 | TBC1 domain family | 1.504847821 | NAGA | alpha-N- | −4.074676686 |
| | member 7 | | | acetylgalactosaminidase | |
| ARSB | arylsulfatase B | 1.504845728 | KLRA20 | killer cell lectin-like | −4.078951341 |
| | | | | receptor subfamily A, | |
| | | | | member 20(Klra20) | |
| RAD17 | RAD17 checkpoint clamp | 1.504177542 | STK11IP | serine/threonine kinase 11 | −4.083213368 |
| | loader component | | | interacting protein | |
| CMTM7 | CKLF like MARVEL | 1.503297831 | KLF4 | Kruppel like factor 4 | −4.084306687 |
| | transmembrane domain | | | | |
| | containing 7 | | | | |
| NFKB2 | nuclear factor kappa B | 1.500363085 | INADL | Description Not Found | −4.086667018 |
| | subunit 2 | | | | |
| TOP3A | topoisomerase (DNA) III | −1.50007357 | URM1 | ubiquitin related modifier 1 | −4.0907078 |
| | alpha | | | | |
| RAB33B | RAB33B, member RAS | −1.50054042 | PELI1 | pellino E3 ubiquitin protein | −4.093813673 |
| | oncogene family | | | ligase 1 | |
| LYSMD1 | LysM domain containing | −1.500614885 | FBLN1 | fibulin 1 | −4.098032083 |
| | 1 | | | | |
| POLG2 | polymerase (DNA) | −1.500707646 | HR | hair growth associated | −4.135452784 |
| | gamma 2, accessory | | | | |
| | subunit | | | | |
| TGIF1 | TGFB induced factor | −1.501196523 | ASB6 | ankyrin repeat and SOCS | −4.137503524 |
| | homeobox 1 | | | box containing 6 | |
| RELL1 | RELT like 1 | −1.50300255 | SLC27A5 | solute carrier family 27 | −4.141596278 |
| | | | | member 5 | |
| CYP26B1 | cytochrome P450 family | −1.50439813 | PPP1R3F | protein phosphatase 1 | −4.14974712 |
| | 26 subfamily B member 1 | | | regulatory subunit 3F | |
| PTRH2 | peptidyl-tRNA hydrolase | −1.504678598 | AB124611 | cDNA sequence | −4.173373402 |
| | 2 | | | AB124611(AB124611) | |
| ZKSCAN3 | zinc finger with KRAB | −1.504916722 | CD40 | CD40 molecule | −4.181897643 |
| | and SCAN domains 3 | | | | |
| SP8 | Sp8 transcription factor | −1.505999092 | SMAD5 | SMAD family member 5 | −4.183883459 |
| SAMD14 | sterile alpha motif domain | −1.506272343 | COL23A1 | collagen type XXIII alpha 1 | −4.221103725 |
| | containing 14 | | | chain | |
| MX2 | MX dynamin like GTPase | −1.507268463 | ZFP595 | zinc finger protein | −4.228818691 |
| | 2 | | | 595(Zfp595) | |
| OCRL | OCRL, inositol | −1.507638755 | PECAM1 | platelet and endothelial cell | −4.232789973 |
| | polyphosphate-5- | | | adhesion molecule 1 | |
| | phosphatase | | | | |
| SYNJ2BP | synaptojanin 2 binding | −1.507669173 | TMEM138 | transmembrane protein 138 | −4.241228289 |
| | protein | | | | |
| CPLX4 | complexin 4 | −1.508554002 | RFX2 | regulatory factor X2 | −4.244125943 |
| LGALS9 | galectin 9 | −1.509246723 | KCTD12 | potassium channel | −4.247846204 |
| | | | | tetramerization domain | |
| | | | | containing 12 | |
| TAZ | tafazzin | −1.509269953 | TRIM56 | tripartite motif containing | −4.262008929 |
| | | | | 56 | |
| 2310002L09RIK | Description Not Found | −1.510961919 | EIF4EBP2 | eukaryotic translation | −4.263034406 |
| | | | | initiation factor 4E binding | |
| | | | | protein 2 | |
| ZFP97 | zinc finger protein | −1.510961919 | RALGPS2 | Ral GEF with PH domain | −4.279842694 |
| | 97(Zfp97) | | | and SH3 binding motif 2 | |
| OLFR1494 | olfactory receptor | −1.510961919 | TGM2 | transglutaminase 2 | −4.293161941 |
| | 1494(Olfr1494) | | | | |
| BC030867 | cDNA sequence | −1.510961919 | ENC1 | ectodermal-neural cortex 1 | −4.311067102 |
| | BC030867(BC030867) | | | | |
| CEACAM9 | carcinoembryonic | −1.510961919 | LRIG1 | leucine rich repeats and | −4.375039431 |
| | antigen-related cell | | | immunoglobulin like | |
| | adhesion molecule | | | domains 1 | |
| | 9(Ceacam9) | | | | |
| LRIT1 | leucine rich repeat, Ig-like | −1.510961919 | PRM1 | protamine 1 | −4.375039431 |
| | and transmembrane | | | | |
| | domains 1 | | | | |
| KLK5 | kallikrein related | −1.510961919 | DUSP7 | dual specificity phosphatase | −4.383538076 |
| | peptidase 5 | | | 7 | |
| KRT27 | keratin 27 | −1.510961919 | SERTAD3 | SERTA domain containing | −4.399171094 |
| | | | | 3 | |
| CACNG4 | calcium voltage-gated | −1.510961919 | KCNC1 | potassium voltage-gated | −4.409390936 |
| | channel auxiliary subunit | | | channel subfamily C | |
| | gamma 4 | | | member 1 | |
| IL13RA1 | interleukin 13 receptor | −1.510961919 | UBE2D3 | ubiquitin conjugating | −4.462706751 |
| | subunit alpha 1 | | | enzyme E2 D3 | |
| TMEM121 | transmembrane protein | −1.510961919 | SEPP1 | selenoprotein P, plasma, 1 | −4.463383458 |
| | 121 | | | | |
| HIST1H2AA | histone cluster 1, H2aa | −1.510961919 | ADRB2 | adrenoceptor beta 2 | −4.463910999 |
| MPZL3 | myelin protein zero like 3 | −1.510961919 | PPP1R13B | protein phosphatase 1 | −4.471417658 |
| | | | | regulatory subunit 13B | |
| TGFB2 | transforming growth | −1.510961919 | ARRDC3 | arrestin domain containing | −4.504620392 |
| | factor beta 2 | | | 3 | |
| IFT74 | intraflagellar transport 74 | −1.510961919 | GNGT2 | G protein subunit gamma | −4.531381461 |
| | | | | transducin 2 | |
| FCRL1 | Fc receptor like 1 | −1.510961919 | SIAH1A | seven in absentia | −4.539158811 |
| | | | | 1A(Siah1a) | |
| ADRB1 | adrenoceptor beta 1 | −1.510961919 | XPC | XPC complex subunit, | −4.563768278 |
| | | | | DNA damage recognition | |
| | | | | and repair factor | |
| MAGI2 | membrane associated | −1.510961919 | HIPK1 | homeodomain interacting | −4.683696454 |
| | guanylate kinase, WW | | | protein kinase 1 | |
| | and PDZ domain | | | | |
| | containing 2 | | | | |
| SCG5 | secretogranin V | −1.510961919 | H2-OB | histocompatibility 2, O | −4.700439718 |
| | | | | region beta locus(H2-Ob) | |
| GCK | glucokinase | −1.510961919 | BACH2 | BTB domain and CNC | −4.716990894 |
| | | | | homolog 2 | |
| ASB10 | ankyrin repeat and SOCS | −1.510961919 | MAPILC3A | microtubule associated | −4.722466024 |
| | box containing 10 | | | protein 1 light chain 3 alpha | |
| SELE | selectin E | −1.510961919 | LRRFIP1 | LRR binding FLII | −4.761551232 |
| | | | | interacting protein 1 | |
| IGFBP3 | insulin like growth factor | −1.510961919 | ATP10D | ATPase phospholipid | −4.766581958 |
| | binding protein 3 | | | transporting 10D (putative) | |
| TPT1 | tumor protein, | −1.510961919 | IGFBP4 | insulin like growth factor | −4.790993785 |
| | translationally-controlled | | | binding protein 4 | |
| | 1 | | | | |
| ROCK1 | Rho associated coiled-coil | −1.510961919 | TMEM108 | transmembrane protein 108 | −4.865423978 |
| | containing protein kinase | | | | |
| | 1 | | | | |
| OGFRL1 | opioid growth factor | −1.510961919 | PTK2 | protein tyrosine kinase 2 | −4.875719796 |
| | receptor-like 1 | | | | |
| TMEM38A | transmembrane protein | −1.510961919 | CLEC11A | C-type lectin domain family | −4.897240426 |
| | 38A | | | 11 member A | |
| RLTPR | Description Not Found | −1.51227339 | LRP12 | LDL receptor related | −4.955029571 |
| | | | | protein 12 | |
| ITPKC | inositol-trisphosphate 3- | −1.512389725 | GCNT2 | glucosaminyl (N-acetyl) | −4.958842675 |
| | kinase C | | | transferase 2, I-branching | |
| | | | | enzyme (I blood group) | |
| TLE4 | transducin like enhancer | −1.51341989 | F10 | coagulation factor X | −4.965784285 |
| | of split 4 | | | | |
| PDE4D | phosphodiesterase 4D | −1.513667908 | DBP | D-box binding PAR bZIP | −4.966549451 |
| | | | | transcription factor | |
| A130010J15RIK | Description Not Found | −1.514296211 | ABCG1 | ATP binding cassette | −5.002252452 |
| | | | | subfamily G member 1 | |
| RNF167 | ring finger protein 167 | −1.514765492 | WDR78 | WD repeat domain 78 | −5.017921908 |
| CCBL1 | Description Not Found | −1.515626494 | DNAJC6 | DnaJ heat shock protein | −5.017921908 |
| | | | | family (Hsp40) member C6 | |
| HSD17B1 | hydroxysteroid 17-beta | −1.516875069 | AFF4 | AF4/FMR2 family member | −5.033423002 |
| | dehydrogenase 1 | | | 4 | |
| OSM | oncostatin M | −1.517234668 | TNFRSF26 | tumor necrosis factor | −5.040015679 |
| | | | | receptor superfamily, | |
| | | | | member 26(Tnfrsf26) | |
| RHPN1 | rhophilin, Rho GTPase | −1.517275693 | GFOD2 | glucose-fructose | −5.070389328 |
| | binding protein 1 | | | oxidoreductase domain | |
| | | | | containing 2 | |
| TAS2R105 | taste receptor, type 2, | −1.517431856 | TYROBP | TYRO protein tyrosine | −5.114783447 |
| | member 105(Tas2r105) | | | kinase binding protein | |
| NIPBL | NIPBL, cohesin loading | −1.517569618 | TMEM176B | transmembrane protein | −5.118941073 |
| | factor | | | 176B | |
| CXCR3 | C-X-C motif chemokine | −1.519325267 | ZFP710 | zinc finger protein | −5.159871337 |
| | receptor 3 | | | 710(Zfp710) | |
| SMURF1 | SMAD specific E3 | −1.520263252 | ENPP4 | ectonucleotide | −5.181897643 |
| | ubiquitin protein ligase 1 | | | pyrophosphatase/ | |
| | | | | phosphodiesterase 4 | |
| | | | | (putative) | |
| RNF208 | ring finger protein 208 | −1.52126647 | MAPK8 | mitogen-activated protein | −5.259272487 |
| | | | | kinase 8 | |
| ITGA5 | integrin subunit alpha 5 | −1.523517983 | TNFRSF25 | tumor necrosis factor | −5.289096702 |
| | | | | receptor superfamily | |
| | | | | member 25 | |
| USP18 | ubiquitin specific | −1.524814077 | LCN4 | lipocalin 4(Lcn4) | −5.366322214 |
| | peptidase 18 | | | | |
| PIP5K1A | phosphatidylinositol-4- | −1.525074369 | CRIM1 | cysteine rich | −5.369815424 |
| | phosphate 5-kinase type 1 | | | transmembrane BMP | |
| | alpha | | | regulator 1 | |
| STRBP | spermatid perinuclear | −1.52561213 | RTP4 | receptor transporter protein | −5.444600814 |
| | RNA binding protein | | | 4 | |
| GRAMD2 | GRAM domain | −1.52652805 | PRNP | prion protein | −5.495055528 |
| | containing 2 | | | | |
| ZFP101 | zinc finger protein | −1.526555668 | ZFP747 | zinc finger protein | −5.496654083 |
| | 101(Zfp101) | | | 747(Zfp747) | |
| RUNDC1 | RUN domain containing 1 | −1.526563287 | CD7 | CD7 molecule | −5.504620392 |
| SLC13A3 | solute carrier family 13 | −1.528487927 | ARHGAP26 | Rho GTPase activating | −5.548436625 |
| | member 3 | | | protein 26 | |
| CCDC94 | coiled-coil domain | −1.528487927 | S100A9 | S100 calcium binding | −5.557655155 |
| | containing 94 | | | protein A9 | |
| MRPS14 | mitochondrial ribosomal | −1.528962318 | AQP9 | aquaporin 9 | −5.572889668 |
| | protein S14 | | | | |
| NEU4 | neuraminidase 4 | −1.529820947 | CXCR5 | C-X-C motif chemokine | −5.573647187 |
| | (sialidase) | | | receptor 5 | |
| PCGF1 | polycomb group ring | −1.53059536 | CCNO | cyclin O | −5.574404309 |
| | finger 1 | | | | |
| PNPLA7 | patatin like phospholipase | −1.53207883 | LYNX1 | Ly6/neurotoxin 1 | −5.666756592 |
| | domain containing 7 | | | | |
| SPATA19 | spermatogenesis | −1.533014103 | CLDN10 | claudin 10 | −5.782015335 |
| | associated 19 | | | | |
| AP4B1 | adaptor related protein | −1.533821865 | AMIGO2 | adhesion molecule with Ig- | −5.83541884 |
| | complex 4 beta 1 subunit | | | like domain 2 | |
| BC068281 | cDNA sequence | −1.5360529 | CD79B | CD79b molecule | −5.94016675 |
| | BC068281(BC068281) | | | | |
| GK2 | glycerol kinase 2 | −1.5360529 | USP53 | ubiquitin specific peptidase | −5.980710829 |
| | | | | 53 | |
| PIGM | phosphatidylinositol | −1.5360529 | IKBKE | inhibitor of kappa light | −6.005624549 |
| | glycan anchor | | | polypeptide gene enhancer | |
| | biosynthesis class M | | | in B-cells, kinase epsilon | |
| FKBP6 | FK506 binding protein 6 | −1.5360529 | ALOX5AP | arachidonate 5- | −6.008988783 |
| | | | | lipoxygenase activating | |
| | | | | protein | |
| EVI5 | ecotropic viral integration | −1.5360529 | GGT1 | gamma-glutamyltransferase | −6.010108453 |
| | site 5 | | | 1 | |
| BCL11A | B-cell CLL/lymphoma | −1.5360529 | CAMK2D | calcium/calmodulin | −6.047669251 |
| | 11A | | | dependent protein kinase II | |
| | | | | delta | |
| PER1 | period circadian clock 1 | −1.537278499 | RAB3D | RAB3D, member RAS | −6.156841525 |
| | | | | oncogene family | |
| BTBD9 | BTB domain containing 9 | −1.537451456 | MAP3K8 | mitogen-activated protein | −6.376776572 |
| | | | | kinase kinase kinase 8 | |
| USP38 | ubiquitin specific | −1.537763627 | NOTCH4 | notch 4 | −6.495055528 |
| | peptidase 38 | | | | |
| LRRC57 | leucine rich repeat | −1.538083341 | MACROD1 | MACRO domain | −6.581200582 |
| | containing 57 | | | containing 1 | |
| 5830415F09RIK | Description Not Found | −1.53855912 | RNF144A | ring finger protein 144A | −6.632268216 |
| EGR2 | early growth response 2 | −1.540038325 | PDE2A | phosphodiesterase 2A | −6.86913112 |
| GMEB2 | glucocorticoid | −1.541122795 | THA1 | threonine aldolase 1(Tha1) | −6.885086225 |
| | modulatory element | | | | |
| | binding protein 2 | | | | |
| PIK3R4 | phosphoinositide-3-kinase | −1.541975323 | APP | amyloid beta precursor | −6.940754047 |
| | regulatory subunit 4 | | | protein | |
| KRR1 | KRR1, small subunit | −1.54225805 | FAM109A | family with sequence | −6.968666793 |
| | processome component | | | similarity 109 member A | |
| | homolog | | | | |
| COL9A1 | collagen type IX alpha 1 | −1.54225805 | LRG1 | leucine rich alpha-2- | −6.995484519 |
| | | | | glycoprotein 1 | |
| POLD4 | polymerase (DNA) delta | −1.542654605 | IL11RA1 | interleukin 11 receptor, | −7.016251155 |
| | 4, accessory subunit | | | alpha chain 1(Il11ra1) | |
| ACSS2 | acyl-CoA synthetase | −1.544045378 | CNR2 | cannabinoid receptor 2 | −7.213347282 |
| | short-chain family | | | | |
| | member 2 | | | | |
| PDLIM1 | PDZ and LIM domain 1 | −1.544785186 | NUAK2 | NUAK family kinase 2 | −7.369815424 |
| A430107P09RIK | Description Not Found | −1.544921568 | GPR146 | G protein-coupled receptor | −7.577806447 |
| | | | | 146 | |
| SLC38A11 | solute carrier family 38 | −1.546222547 | | | |
| | member 11 | | | | |

*Log2Fold Change = log2(4 + L+/4 − L−)

To investigate the molecular pathways between these three populations, gene ontology networks were grouped into nodes and the most significant pathways within each node were determined (FIG. 6A). Gene ontology (GO) terms shared between our dysfunctional T cell dataset and the published hypofunctional T cell dataset were greatly enriched in cell cycle genes, consistent with the observation that the dysfunctional population is largely Ki67⁺. GO terms shared between dysfunctional and exhausted gene sets encompassed effector programs such as regulation of cell killing, chemotaxis, interferon-γ production. GO terms shared between hypofunctional and exhausted gene sets consisted of cell cycle pathways, negative regulation of lymphocytes, and interferon-γ production. These data indicate that while some conserved molecular programs likely exist in these dysfunctional differentiation states, many pathways may be differentially regulated between chronic viral infections and in the tumor context. While many inhibitory receptors, including Pdcd1 (PD-1), Havcr2 (TIM-3), Cd244 (2B4), Klrel, and Lag3 were shared between all data sets; the co-stimulatory receptors Tnfrsf4 (OX-40) and Tnfrsf9 (4-1BB) were upregulated in dysfunctional and hypofunctional CD8⁺ TIL data sets. Therefore, to enrich in potential markers and therapeutic targets on tumor specific CD8⁺ TILs, the complete cell surface phenotype of the 4-1BB⁺LAG-3⁺ CD8 TIL population was characterized. Comparing the different CD8⁺ TIL subpopulations, several additional upregulated co-stimulatory receptors were found: Tnfrsf18 (GITR), Nkg2d (KLRK1) and Cd27. The transcript for Nrp1 (neuropilin-1), which encodes for a cell surface receptor protein implicated in CD4⁺ Treg function (Sarris et al., 2008; incorporated by reference in its entirety), was also highly expressed. Expression of many of these molecules was confirmed by flow cytometry at day 7, 14 and 21 after tumor inoculation (FIG. 6C). The analysis was extended to include the co-stimulatory molecules ICOS and CD160 and the inhibitory receptor T cell immunoreceptor with Ig and ITIM domains (TIGIT) because ICOS and CD160 were close to the cutoff value and no probe was present for TIGIT in the gene array. In addition, recent reports indicate that targeting these receptors can be therapeutic in murine models of cancer (Johnston et al., 2014; Fan et al., 2014; incorporated by reference in their entireties). PD-1, TIGIT, TIM-3, CD27 and NRP1 were expressed the majority of the 4-1BB⁺LAG-3⁺ TIL population and expression was maintained over time. 2B4, CD160, CTLA4, OX-40, and GITR subdivided a lesser fraction of the 4-1BB⁺LAG-3⁺ population. The expression of several inhibitory receptors, 2B4, TIM3 and CD160 increased over this 3-week time frame while expression of the co-stimulatory receptors, ICOS and OX-40, decreased (FIG. 6C).

To address if the dysfunctional CD8⁺ TILs are terminally-differentiated short term effector cells or memory-like cells, the expression of KLRG-1 and IL-7Ra (Joshi et al., 2007). Most of the CD8⁺ TIL were negative for KLRG-1 expression and there was no difference between the 4-1BB⁺LAG-3⁺ and 4-1BB⁻LAG-3⁻ populations. However, the majority of the 4-1BB⁺LAG-3⁺ TIL did not express the IL-7 receptor (IL-7Rα) compared to their negative counter parts (FIG. 6D). These results indicate that the 4-1BB⁻LAG-3⁻TIL, which are not apparently specific for antigens expressed in the tumor microenvironment, are more memory-like, yet at the same time the tumor antigen-specific LAG-3⁺4-1BB⁺ subset has not fully acquired a terminal effector phenotype.

Functional Relevance of Genes that are Differentially Regulated in CD8⁺4-1BB⁺LAG-3⁺ TILs

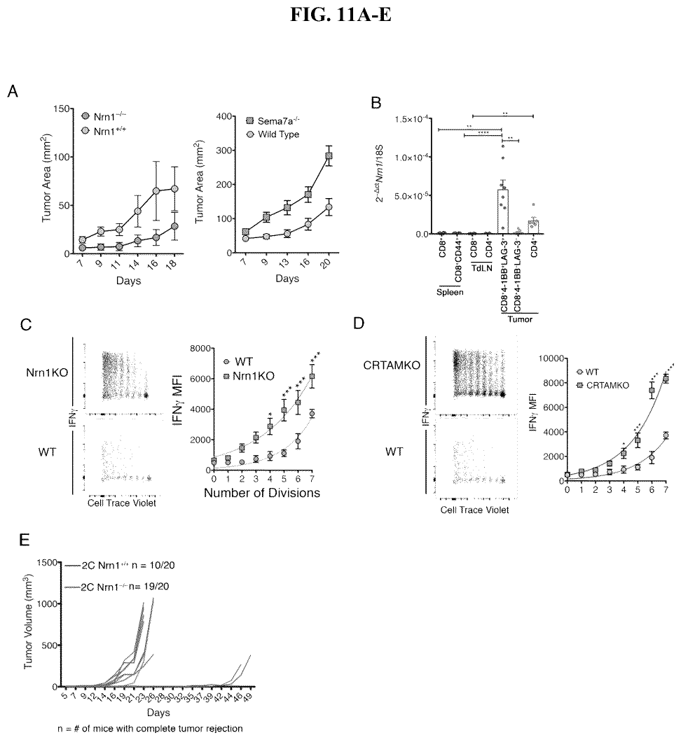

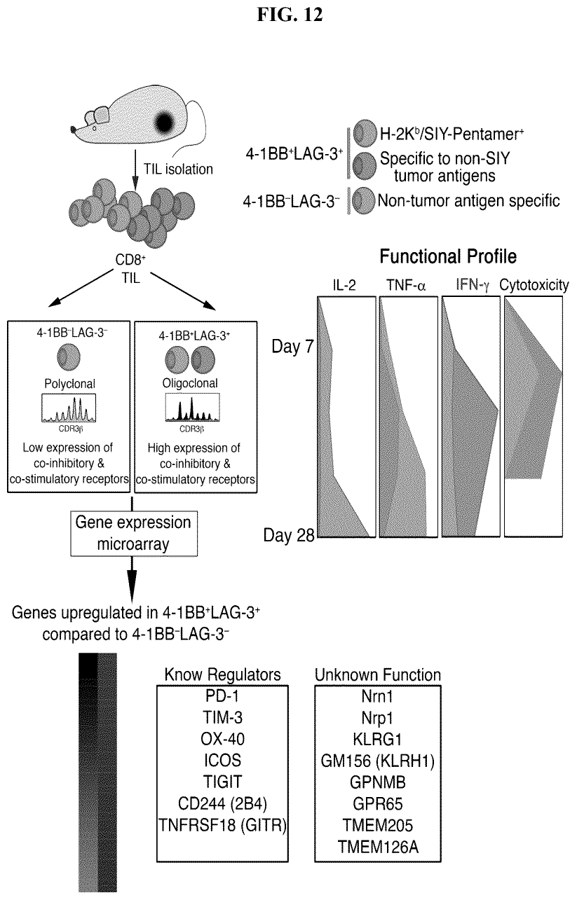

The gene array results in Table 2 provide a list of genes characterizing CD8⁺4-1BB⁺LAG-3⁺ TILs. The list includes therapeutic targets and additional markers of anti-tumor immunity. Experiments conducted during development of embodiments herein to test the functional relevance of these additional targets/markers (FIG. 11). Data indicate that the array has identified targets for immunotherapy, using knockout mice (e.g., PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM, Sema7a, etc.). Experiments demonstrate that Nrn1 and CRTAM are negative regulators of the anti-tumor immune response, as knockout mice lacking either of these molecules showed improved immune-mediated tumor control in vivo. In contrast, Sema7a is a positive regulator of anti-tumor immune responses, as knockout mice lacking this molecule show diminished immune-mediated tumor control in vivo (FIG. 11). These experiments indicate that agonists of Sema7a signaling and antagonists of Nrn1 and/or CRTAM should be useful therapeutics for the treatment of cancer.

### Targeting 4-1BB and LAG-3 Exerts Anti-Tumor Activity In Vivo and Normalizes the Function and Phenotypic Composition of CD8⁺ TILs

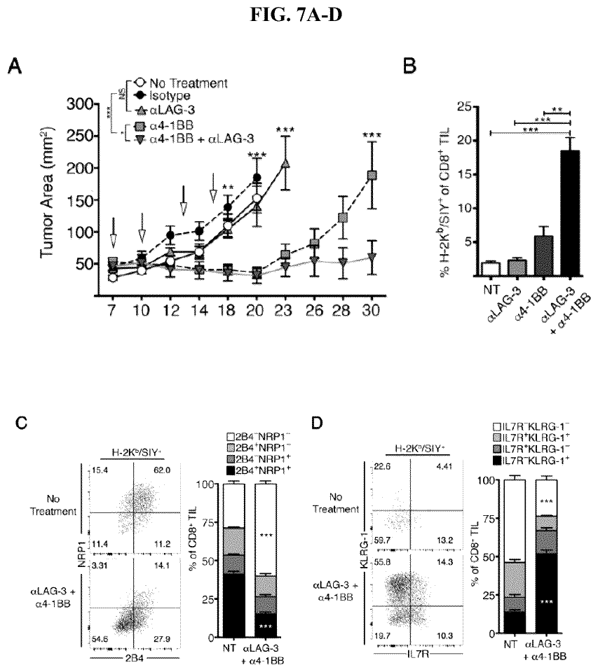

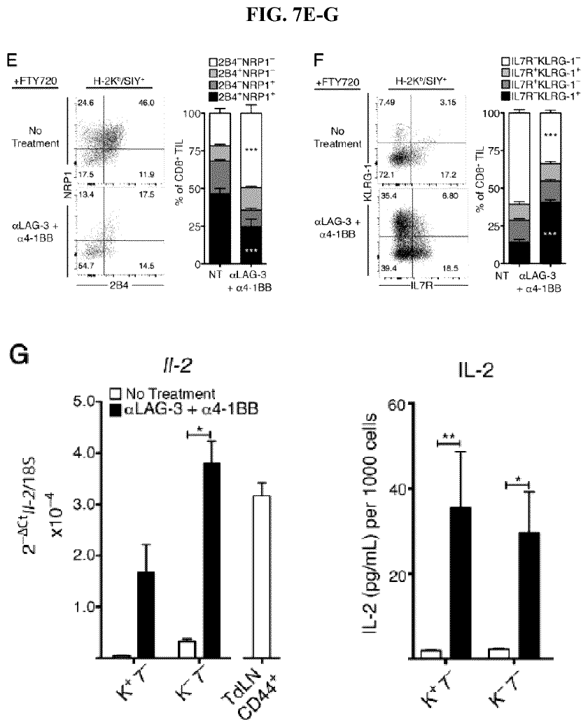

Experiments were conducted during development of embodiments herein to assess whether targeting these receptors might have therapeutic utility. To this end, an agonistic anti-4-1BB mAb was administered alone or in combination with a blocking anti-LAG-3 mAb in mice bearing established B16.SIY tumors. While each antibody treatment alone had some therapeutic effect as reflected by slower tumor growth, the combination was particularly potent (FIG. 7A). Analysis of the tumor microenvironment revealed that improved tumor control with the combination therapy was accompanied by an increase in the number of CD8⁺ TILs specific for the SIY antigen (FIG. 7B), consistent with results reported previously with anti-PD-L1+anti-CTLA-4 mAb (Spranger et al., 2014b; Twyman-Saint Victor et al., 2015; incorporated by reference in their entireties).

It was next examined whether the therapeutic effect of anti-4-1BB+anti-LAG-3 mAbs was associated with a loss of phenotypic markers defining dysfunctional T cells in the steady state. Due to concern that re-analyzing the T cells for expression of LAG-3 and 4-1BB might be problematic, as the administered Abs could theoretically modulate the target receptors from the cell surface, the coordinate expression of additional receptors as identified above by gene expression profiling was taken advantage of. Preliminary analyses of the bulk TIL subpopulations revealed decreased expression of NRP1 and 2B4 following anti-LAG-3+anti-4-1BB treatment (data not shown). Co-expression of 2B4 and NRP1 on SIY-reactive CD8⁺ TILs identified by pentamer staining was analyzed. A 2.7-fold-decrease in the co-expression of 2B4 and NRP1 was observed upon anti-4-1BB⁺ and anti-LAG-3 mAb treatment (FIG. 7C), indicating a loss of the surface phenotype associated with T cell dysfunction. To determine whether this change was accompanied by a shift towards an effector phenotype, expression of KLGR-1 was examined. Indeed, a marked increase in KLGR-1 expression was observed on the SIY-reactive TIL following treatment, and a 3.7-fold increase in the KLRG-11^{hi}IL-7RA^{lo} population was observed (FIG. 7D).

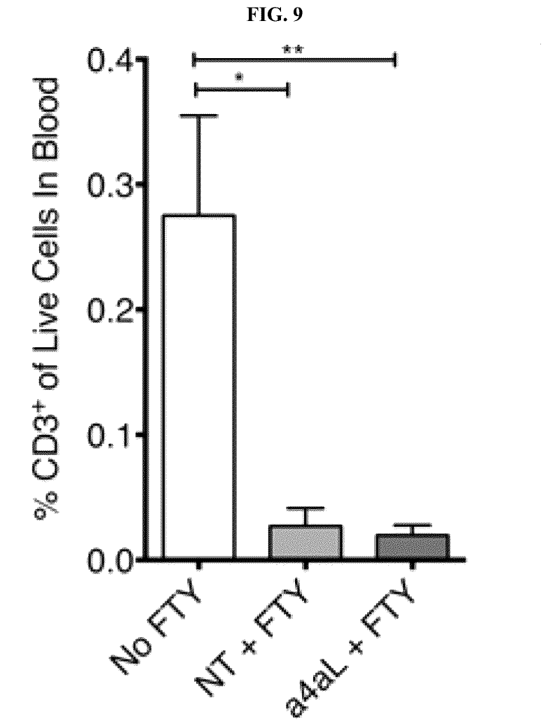

To eliminate the possibility that treatment with anti-LAG-3+anti-4-1BB mAbs was not altering the phenotype of T cells already within the tumor but rather was supporting recruitment of newly primed functional T cells from secondary lymphoid organs, the SlPR inhibitor FTY720, which prevents T cell egress from lymph nodes (Halin et al., 2005; incorporated by reference in its entirety), was utilized. We The efficacy of anti-PD-L1-based immunotherapies was preserved in the presence of FTY720, arguing for re-functionalization of TIL as the major mechanism of action (Spranger et al., 2014a; incorporated by reference in its entirety). FTY720 administration was started on day 6 after tumor inoculation, 24 hours before the start of anti-LAG-3+anti-4-1BB treatment, and continued every day until TIL analysis on day 14. Peripheral blood analyzed at the same time point revealed marked depletion of circulating T cells (FIG. 9). Despite this loss of circulating T cells, the down regulation of 2B4 and NRP1 and the shift towards the KLRG1^{hi}IL-7RA^{lo} phenotype was nonetheless preserved (FIGS. 7E and F). To examine functional restoration of the TIL, the KLRG-^{lo}IL-7RA^{lo} and KLRG-1^{hi} IL-7RA^{lo} CD8⁺ TIL populations were sorted from B16.SIY tumors on day 14 following treatment and analyzed for IL-2 after restimulation in vitro. Indeed, the KLRG-^{lo}IL-7RA^{lo} and KLRG-1^{hi}IL-7RA^{lo} populations showed an increased capacity to produce IL-2 upon stimulation (FIG. 7G). The relative level of Il-2 mRNA was comparable between the two CD8⁺ TIL populations and control CD8⁺CD44⁺ TdLN T cells. Collectively, these data indicate that anti-4-1BB/anti-LAG-3 combinatorial treatment induces significant changes in the phenotype profile and promotes functional restoration of tumor antigen-specific CD8⁺ T cells already present within the tumor microenvironment.

### REFERENCES

The following references, some of which are cited above, are herein incorporated by references in their entireties.
Ahmadzadeh, M., L. A. Johnson, B. Heemskerk, J. R. Wunderlich, M. E. Dudley, D. E. White, and S. A. Rosenberg. 2009. Tumor antigen-specific CD8 T cells infiltrating the tumor express high levels of PD-1 and are functionally impaired. Blood. 114:1537-1544. doi:10.1182/blood-2008-12-195792.
Baitsch, L., A. Legat, L. Barba, S. A. Fuertes Marraco, J.-P. Rivals, P. Baumgaertner, C. Christiansen-Jucht, H. Bouzourene, D. Rimoldi, H. Pircher, N. Rufer, M. Matter, O. Michielin, and D. E. Speiser. 2012. Extended co-expression of inhibitory receptors by human CD8 T-cells depending on differentiation, antigen-specificity and anatomical localization. PLoS ONE. 7:e30852. doi:10.1371/journal.pone.0030852.
Baitsch, L., P. Baumgaertner, E. Devêvre, S. K. Raghav, A. Legat, L. Barba, S. Wieckowski, H. Bouzourene, B. Deplancke, P. Romero, N. Rufer, and D. E. Speiser. 2011. Exhaustion of tumor-specific CD8⁺ T cells in metastases from melanoma patients. J. Clin. Invest. 121:2350-2360. doi:10.1172/JC146102.
Blackburn, S. D., H. Shin, W. N. Haining, T. Zou, C. J. Workman, A. Polley, M. R. Betts, G. J. Freeman, D. A. A. Vignali, and E. J. Wherry. 2009. Coregulation of CD8+ T cell exhaustion by multiple inhibitory receptors during chronic viral infection. Nature Publishing Group. 10:29-37. doi:10.1038/ni.1679.
Brown, I. E., C. Blank, J. Kline, A. K. Kacha, and T. F. Gajewski. 2006. Homeostatic proliferation as an isolated variable reverses CD8+ T cell anergy and promotes tumor rejection. Journal of Immunology (Baltimore, Md.: 1950). 177:4521-4529.
Clouthier, D. L., A. C. Zhou, and T. H. Watts. 2014. Anti-GITR agonist therapy intrinsically enhances CD8 T cell responses to chronic lymphocytic choriomeningitis virus (LCMV), thereby circumventing LCMV-induced downregulation of costimulatory GITR ligand on APC. The Journal of Immunology. 193:5033-5043. doi:10.4049/jimmunol.1401002.
Crawford, A., J. M. Angelosanto, C. Kao, T. A. Doering, P. M. Odorizzi, B. E. Barnett, and E. J. Wherry. 2014. Molecular and Transcriptional Basis of CD4(+) T Cell Dysfunction during Chronic Infection. Immunity. 40:289-302. doi:10.1016/j.immuni.2014.01.005.
Cunningham, C. R., A. Champhekar, M. V. Tullius, B. J. Dillon, A. Zhen, J. R. de la Fuente, J. Herskovitz, H. Elsaesser, L. M. Snell, E. B. Wilson, J. C. de la Torre, S. G. Kitchen, M. A. Horwitz, S. J. Bensinger, S. T. Smale, and D. G. Brooks. 2016. Type I and Type II Interferon Coordinately Regulate Suppressive Dendritic Cell Fate and Function during Viral Persistence. PLoS Pathog. 12:e1005356. doi:10.1371/journal.ppat.1005356.
Currier, J. R., and M. A. Robinson. 2001. Spectratype/Immunoscope Analysis of the Expressed TCR Repertoire. John Wiley & Sons, Inc., Hoboken, NJ, USA. 92544 pp.
Diamond, M. S., M. Kinder, H. Matsushita, M. Mashayekhi, G. P. Dunn, J. M. Archambault, H. Lee, C. D. Arthur, J. M. White, U. Kalinke, K. M. Murphy, and R. D. Schreiber. 2011. Type I interferon is selectively required by dendritic cells for immune rejection of tumors. J. Exp. Med. 208:1989-2003. doi:10.1084/jem.20101158.
Doering, T. A., A. Crawford, J. M. Angelosanto, M. A. Paley, C. G. Ziegler, and E. J. Wherry. 2012. Network analysis reveals centrally connected genes and pathways involved in CD8+ T cell exhaustion versus memory. Immunity. 37:1130-1144. doi:10.1016/j.immuni.2012.08.021.
Evaristo, C., S. Spranger, S. E. Barnes, M. L. Miller, L. L. Molinero, F. L. Locke, T. F. Gajewski, and M.-L. Alegre. 2016. Cutting Edge: Engineering Active IKKβ in T Cells Drives Tumor Rejection. The Journal of Immunology. 196:2933-2938. doi:10.4049/jimmunol.1501144.
Fan, X., S. A. Quezada, M. A. Sepulveda, P. Sharma, and J. P. Allison. 2014. Engagement of the ICOS pathway markedly enhances efficacy of CTLA-4 blockade in cancer immunotherapy. J. Exp. Med. 211:715-725. doi:10.1084/jem.20130590.
Fourcade, J., Z. Sun, O. Pagliano, P. Guillaume, I. F. Luescher, C. Sander, J. M. Kirkwood, D. Olive, V. Kuchroo, and H. M. Zarour. 2012. CD8(+) T cells specific for tumor antigens can be rendered dysfunctional by the tumor microenvironment through upregulation of the inhibitory receptors BTLA and PD-1. Cancer Res. 72:887-896. doi:10.1158/0008-5472.CAN-11-2637.
Fridman, W. H., F. Pages, C. Sautés-Fridman, and J. Galon. 2012. The immune contexture in human tumours: impact on clinical outcome. Nature Publishing Group. 12:298-306. doi:10.1038/nrc3245.
Fuertes, M. B., A. K. Kacha, J. Kline, S.-R. Woo, D. M. Kranz, K. M. Murphy, and T. F. Gajewski. 2011. Host type I IFN signals are required for antitumor CD8+ T cell responses through CD8{alpha}+dendritic cells. J. Exp. Med. 208:2005-2016. doi:10.1084/jem.20101159.
Gajewski, T. F. 2007a. The expanding universe of regulatory T cell subsets in cancer. Immunity. 27:185-187. doi:10.1016/j.immuni.2007.08.001.
Gajewski, T. F. 2007b. Failure at the Effector Phase: Immune Barriers at the Level of the Melanoma Tumor Microenvironment. Clin. Cancer Res. 13:5256-5261. doi:10.1158/1078-0432.CCR-07-0892.
Gajewski, T. F., H. Schreiber, and Y.-X. Fu. 2013. Innate and adaptive immune cells in the tumor microenvironment. Nat Immunol. 14:1014-1022. doi:10.1038/ni.2703.
Gajewski, T. F., Y. Meng, C. Blank, I. E. Brown, A. K. Kacha, J. Kline, and H. Harlin. 2006. Immune resistance orchestrated by the tumor microenvironment. Immunological Reviews. 1-15.
Goldszmid, R. S., A. Dzutsev, and G. Trinchieri. 2014. Host Immune Response to Infection and Cancer: Unexpected Commonalities. Cell Host and Microbe. 15:295-305. doi:10.1016/j.chom.2014.02.003.
Gros, A., P. F. Robbins, X. Yao, Y. F. Li, S. Turcotte, E. Tran, J. R. Wunderlich, A. Mixon, S. Farid, M. E. Dudley, K.-I. Hanada, J. R. Almeida, S. Darko, D. C. Douek, J. C. Yang, and S. A. Rosenberg. 2014. PD-1 identifies the patient-specific CD8⁺ tumor-reactive repertoire infiltrating human tumors. J. Clin. Invest. 124:2246-2259. doi:10.1172/JCI73639.
Halin, C., M. L. Scimone, R. Bonasio, J.-M. Gauguet, T. R. Mempel, E. Quackenbush, R. L. Proia, S. Mandala, and U. H. von Andrian. 2005. The SiP-analog FTY720 differentially modulates T-cell homing via HEV: T-cell-expressed SlPl amplifies integrin activation in peripheral lymph nodes but not in Peyer patches. Blood. 106:1314-1322. doi:10.1182/blood-2004-09-3687.
Harlin, H., T. V. Kuna, A. C. Peterson, Y. Meng, and T. F. Gajewski. 2006. Tumor progression despite massive influx of activated CD8+ T cells in a patient with malignant melanoma ascites. Cancer Immunol Immunother. 55:1185-1197. doi:10.1007/s00262-005-0118-2.
Harlin, H., Y. Meng, A. C. Peterson, Y. Zha, M. Tretiakova, C. Slingluff, M. McKee, and T. F. Gajewski. 2009. Chemokine expression in melanoma metastases associated with CD8+ T-cell recruitment. Cancer Res. 69:3077-3085. doi:10.1158/0008-5472.CAN-08-2281.
Hoelzinger, D. B., S. E. Smith, N. Mirza, A. L. Dominguez, S. Z. Manrique, and J. Lustgarten. 2010. Blockade of CCL1 Inhibits T Regulatory Cell Suppressive Function Enhancing Tumor Immunity without Affecting T Effector Responses. The Journal of Immunology. 184:6833-6842. doi:10.4049/jimmunol.0904084.
Jenkins, M. K., D. M. Pardoll, J. Mizuguchi, T. M. Chused, and R. H. Schwartz. 1987. Molecular events in the induction of a nonresponsive state in interleukin 2-producing helper T-lymphocyte clones. PNAS. 84:5409-5413.
Johnston, R. J., L. Comps-Agrar, J. Hackney, X. Yu, M. Huseni, Y. Yang, S. Park, V. Javinal, H. Chiu, B. Irving, D. L. Eaton, and J. L. Grogan. 2014. The immunoreceptor TIGIT regulates antitumor and antiviral CD8(+) T cell effector function. Cancer Cell. 26:923-937. doi:10.1016/j.ccell.2014.10.018.
Joshi, N. S., W. Cui, A. Chandele, H. K. Lee, D. R. Urso, J. Hagman, L. Gapin, and S. M. Kaech. 2007. Inflammation Directs Memory Precursor and Short-Lived Effector CD8+ T Cell Fates via the Graded Expression of T-bet Transcription Factor. Immunity. 27:281-295. doi:10.1016/j.immuni.2007.07.010.
Kaech, S. M., J. T. Tan, E. J. Wherry, B. T. Konieczny, C. D. Surh, and R. Ahmed. 2003. Selective expression of the interleukin 7 receptor identifies effector CD8 T cells that give rise to long-lived memory cells. Nat Immunol. 4:1191-1198. doi:10.1038/nil009.
Kearse, M., R. Moir, A. Wilson, S. Stones-Havas, M. Cheung, S. Sturrock, S. Buxton, A. Cooper, S. Markowitz, C. Duran, T. Thierer, B. Ashton, P. Meintjes, and A. Drummond. 2012. Geneious Basic: an integrated and extendable desktop software platform for the organization and analysis of sequence data. Bioinformatics. 28:1647-1649. doi:10.1093/bioinformatics/bts199.
Kim, J.-O., H. W. Kim, K.-M. Baek, and C.-Y. Kang. 2003. NF-xB and AP-1 regulate activation-dependent CD137 (4-1BB) expression in T cells. FEBS Letters. 541:163-170. doi:10.1016/50014-5793(03)00326-0.
Kline, J., L. Zhang, L. Battaglia, K. S. Cohen, and T. F. Gajewski. 2012. Cellular and molecular requirements for rejection of B16 melanoma in the setting of regulatory T cell depletion and homeostatic proliferation. The Journal of Immunology. 188:2630-2642. doi:10.4049/jimmunol.1100845.
Kuchroo, V. K., A. C. Anderson, and C. Petrovas. 2014. Coinhibitory receptors and CD8 T cell exhaustion in chronic infections. Current Opinion in HIV and AIDS. 9:439-445. doi:10.1097/COH.0000000000000088.
Larkin, J., F. S. Hodi, and J. D. Wolchok. 2015. Combined Nivolumab and Ipilimumab or Monotherapy in Untreated Melanoma. New England Journal of Medicine. 373:1270-1271. doi:10.1056/NEJMc1509660.
Li, S., T. Miao, M. Sebastian, P. Bhullar, E. Ghaffari, M. Liu, A. L. J. Symonds, and P. Wang. 2012. The transcription factors Egr2 and Egr3 are essential for the control of inflammation and antigen-induced proliferation of B and T cells. Immunity. 37:685-696. doi:10.1016/j.immuni.2012.08.001.
Martinez, G. J., R. M. Pereira, T. Aija, E. Y. Kim, F. Marangoni, M. E. Pipkin, S. Togher, V. Heissmeyer, Y. C. Zhang, S. Crotty, E. D. Lamperti, K. M. Ansel, T. R. Mempel, H. Lähdesmäki, P. G. Hogan, and A. Rao. 2015. The Transcription Factor NFAT Promotes Exhaustion of Activated CD8⁺ T Cells. Immunity. 42:265-278. doi:10.1016/j.immuni.2015.01.006.
Odorizzi, P. M., K. E. Pauken, M. A. Paley, A. Sharpe, and E. J. Wherry. 2015. Genetic absence of PD-1 promotes accumulation of terminally differentiated exhausted CD8⁺ T cells. J. Exp. Med. 439:jem.20142237-1137. doi:10.1084/jem.20142237.
Palazón, A., I. Martínez-Forero, A. Teijeira, A. Morales-Kastresana, C. Alfaro, M. F. Sanmamed, J. L. Perez-Gracia, I. Peñuelas, S. Hervás-Stubbs, A. Rouzaut, M. O. de Landázuri, M. Jure-Kunkel, J. Aragonés, and I. Melero. 2012. The HIF-1α hypoxia response in tumor-infiltrating T lymphocytes induces functional CD137 (4-1BB) for immunotherapy. Cancer Discov. 2:608-623. doi:10.1158/2159-8290.CD-11-0314.
Pardoll, D. M. 2012. The blockade of immune checkpoints in cancer immunotherapy. 1-13. doi:10.1038/nrc3239.
Pauken, K. E., and E. J. Wherry. 2015. Overcoming T cell exhaustion in infection and cancer. Trends in Immunology. 36:265-276. doi:10.1016/j.it.2015.02.008.
Pearce, E. L., M. C. Poffenberger, C. H. Chang, and R. G. Jones. 2013. Fueling immunity: insights into metabolism and lymphocyte function. Science.
Plaisier, S. B., R. Taschereau, J. A. Wong, and T. G. Graeber. 2010. Rank-rank hypergeometric overlap: identification of statistically significant overlap between gene-expression signatures. Nucl. Acids Res. 38:e169-el69. doi:10.1093/nar/gkg636.
Rosenblatt, J. D., and J. L. Stein. 2014. RRHO: Test overlap using the Rank-Rank Hypergeometric test.
Safford, M., S. Collins, M. A. Lutz, A. Allen, C.-T. Huang, J. Kowalski, A. Blackford, M. R. Horton, C. Drake, R. H. Schwartz, and J. D. Powell. 2005. Egr-2 and Egr-3 are negative regulators of T cell activation. Nat Immunol. 6:472-480. doi:10.1038/nil193.
Sarkar, S., V. Kalia, W. N. Haining, B. T. Konieczny, S. Subramaniam, and R. Ahmed. 2008. Functional and genomic profiling of effector CD8 T cell subsets with distinct memory fates. J. Exp. Med. 205:625-640. doi:10.1084/jem.20071641.
Sarris, M., K. G. Andersen, F. Randow, L. Mayr, and A. G. Betz. 2008. Neuropilin-1 Expression on Regulatory T Cells Enhances Their Interactions with Dendritic Cells during Antigen Recognition. Immunity. 28:402-413. doi:10.1016/j.immuni.2008.01.012.
Schietinger, A., and P. D. Greenberg. 2014. Tolerance and exhaustion: defining mechanisms of T cell dysfunction. Trends in Immunology. 35:51-60. doi:10.1016/j.it.2013.10.001.
Schietinger, A., M. Philip, V. E. Krisnawan, E. Y. Chiu, J. J. Delrow, R. S. Basom, P. Lauer, D. G. Brockstedt, S. E. Knoblaugh, G. J. Hammerling, T. D. Schell, N. Garbi, and P. D. Greenberg. 2016. Tumor-Specific T Cell Dysfunction Is a Dynamic Antigen-Driven Differentiation Program Initiated Early during Tumorigenesis. Immunity. doi:10.1016/j.immuni.2016.07.011.
Schwartz, R. H. 2003. T C ELLA NERGY*. Annu. Rev. Immunol. 21:305-334. doi:10.1146/annurev.immunol.21.120601.141110.
Schwartz, R. H., D. L. Mueller, M. K. Jenkins, and H. Quill. 1989. T-cell clonal anergy. Cold Spring Harb. Symp. Quant. Biol. 54 Pt 2:605-610.
Shannon, P., A. Markiel, O. Ozier, N. S. Baliga, J. T. Wang, D. Ramage, N. Amin, B. Schwikowski, and T. Ideker. 2003. Cytoscape: a software environment for integrated models of biomolecular interaction networks. Genome Res. 13:2498-2504. doi:10.1101/gr.1239303.
Snell, L. M., and D. G. Brooks. 2015. New insights into type I interferon and the immunopathogenesis of persistent viral infections. Curr. Opin. Immunol. 34:91-98. doi:10.1016/j.coi.2015.03.002.
Spiotto, M. T., P. Yu, D. A. Rowley, M. I. Nishimura, S. C. Meredith, T. F. Gajewski, Y.-X. Fu, and H. Schreiber. 2002. Increasing Tumor Antigen Expression Overcomes “Ignorance” to Solid Tumors via Crosspresentation by Bone Marrow-Derived Stromal Cells. Immunity. 17:737-747. doi:10.1016/51074-7613(02)00480-6.
Spranger, S., H. K. Koblish, and B. Horton. 2014a. Mechanism of tumor rejection with doublets of CTLA-4, PD-1/PD-L1, or IDO blockade involves restored IL-2 production and proliferation of CD8⁺ T cells directly . . . Journal for . . .
Spranger, S., H. K. Koblish, B. Horton, P. A. Scherle, R. Newton, and T. F. Gajewski. 2014b. Mechanism of tumor rejection with doublets of CTLA-4, PD-1/PD-L1, or IDO blockade involves restored IL-2 production and proliferation of CD8(+) T cells directly within the tumor microenvironment. J Immunother Cancer. 2:3. doi:10.1186/2051-1426-2-3.
Spranger, S., R. M. Spaapen, Y. Zha, J. Williams, Y. Meng, T. T. Ha, and T. F. Gajewski. 2013. Up-Regulation of PD-L1, IDO, and Tregs in the Melanoma Tumor Microenvironment Is Driven by CD8⁺ T Cells. Science Translational Medicine. 5:200ra116-200ra116. doi:10.1126/scitranslmed.3006504.
Sumitomo, S., K. Fujio, T. Okamura, and K. Yamamoto. 2013. Egr2 and Egr3 are the unique regulators for systemic autoimmunity. jak-stat. 2:e23952. doi:10.4161/jkst.23952.
Tumeh, P. C., C. L. Harview, J. H. Yearley, I. P. Shintaku, E. J. M. Taylor, L. Robert, B. Chmielowski, M. Spasic, G. Henry, V. Ciobanu, A. N. West, M. Carmona, C. Kivork, E. Seja, G. Cherry, A. J. Gutierrez, T. R. Grogan, C. Mateus, G. Tomasic, J. A. Glaspy, R. O. Emerson, H. Robins, R. H. Pierce, D. A. Elashoff, C. Robert, and A. Ribas. 2014. PD-1 blockade induces responses by inhibiting adaptive immune resistance. Nature. 515:568-571. doi:10.1038/nature13954.
Twyman-Saint Victor, C., A. J. Rech, A. Maity, R. Rengan, K. E. Pauken, E. Stelekati, J. L. Benci, B. Xu, H. Dada, P. M. Odorizzi, R. S. Herati, K. D. Mansfield, D. Patsch, R. K. Amaravadi, L. M. Schuchter, H. Ishwaran, R. Mick, D. A. Pryma, X. Xu, M. D. Feldman, T. C. Gangadhar, S. M. Hahn, E. J. Wherry, R. H. Vonderheide, and A. J. Minn. 2015. Radiation and dual checkpoint blockade activate non-redundant immune mechanisms in cancer. Nature. 520:373-377. doi:10.1038/nature14292.
Vesely, M. D., M. H. Kershaw, R. D. Schreiber, and M. J. Smyth. 2011. Natural Innate and Adaptive Immunity to Cancer. Annu. Rev. Immunol. 29:235-271. doi:10.1146/annurev-immunol-031210-101324.
Waugh, K. A., S. M. Leach, B. L. Moore, T. C. Bruno, J. D. Buhrman, and J. E. Slansky. 2016. Molecular Profile of Tumor-Specific CD8⁺ T Cell Hypofunction in a Transplantable Murine Cancer Model. The Journal of Immunology. 197:1477-1488. doi:10.4049/jimmunol.1600589.
Wherry, E. J., and M. Kurachi. 2015. Molecular and cellular insights into T cell exhaustion. Nat Rev Immunol. 15:486-499. doi:10.1038/nri3862.
Wherry, E. J., S.-J. Ha, S. M. Kaech, W. N. Haining, S. Sarkar, V. Kalia, S. Subramaniam, J. N. Blattman, D. L. Barber, and R. Ahmed. 2007. Molecular signature of CD8⁺ T cell exhaustion during chronic viral infection. Immunity. 27:670-684. doi:10.1016/j.immuni.2007.09.006.
Wu, X., H. Zhang, Q. Xing, J. Cui, J. Li, Y. Li, Y. Tan, and S. Wang. 2014. PD-1(+) CD8(+) T cells are exhausted in tumours and functional in draining lymph nodes of colorectal cancer patients. Br. J. Cancer. 111:1391-1399. doi:10.1038/bjc.2014.416.
Zajac, A. J., J. N. Blattman, K. Murali-Krishna, D. J. Sourdive, M. Suresh, J. D. Altman, and R. Ahmed. 1998. Viral immune evasion due to persistence of activated T cells without effector function. J. Exp. Med. 188:2205-2213. doi:10.1084/jem.188.12.2205.
Zha, Y., R. Marks, A. W. Ho, A. C. Peterson, S. Janardhan, I. Brown, K. Praveen, S. Stang, J. C. Stone, and T. F. Gajewski. 2006. T cell anergy is reversed by active Ras and is regulated by diacylglycerol kinase-alpha. Nat Immunol. 7:1166-1173. doi:10.1038/nil394.
Zhang, L., T. F. Gajewski, and J. Kline. 2009. PD-1/PD-L1 interactions inhibit antitumor immune responses in a murine acute myeloid leukemia model. Blood. 114:1545-1552. doi:10.1182/blood-2009-03-206672.
Zheng, Y., Y. Zha, G. Driessens, F. Locke, and T. F. Gajewski. 2012. Transcriptional regulator early growth response gene 2 (Egr2) is required for T cell anergy in vitro and in vivo. Journal of Experimental Medicine. 209:2157-2163. doi:10.1084/jem.20120342.
Zheng, Y., Y. Zha, R. M. Spaapen, R. Mathew, K. Barr, A. Bendelac, and T. F. Gajewski. 2013. Egr2-dependent gene expression profiling and ChIP-Seq reveal novel biologic targets in T cell anergy. Mol. Immunol. 55:283-291. doi:10.1016/j.molimm.2013.03.006.

1. A method of treating a subject with cancer comprising administering an agent that specifically targets dysfunctional tumor antigen-specific CD8⁺ T cells.

2. The method of claim 1, wherein the subject suffers from a solid tumor cancer.

3. The method of claim 2, wherein the tumor allows T cell infiltration, but is resistant to immunotherapies.

4. The method of claim 2, wherein the tumor environment comprises dysfunctional tumor antigen-specific CD8⁺ T cells.

5. The method of claim 1, comprising contacting the dysfunctional tumor antigen-specific CD8⁺ T cells with an anti-4-1BB and/or anti-LAG3 agent.

6. The method of claim 5, wherein the anti-4-1BB and/or anti-LAG3 agent is an antibody, antibody fragment, or antibody mimetic molecule.

7. The method of claim 1, further comprising co-administration of an additional therapeutic agent.

8. The method of claim 7, wherein the additional therapeutic agent is a chemotherapeutic or an immunotherapeutic agent.

9. The method of claim 8, wherein the additional therapeutic agent is an immunotherapeutic agent selected from the list consisting of cell-based therapies, monoclonal antibody (mAb) therapy, cytokine therapy, and adjuvant treatment.

10. The method of claim 9, wherein the immunotherapeutic agent is a mAb therapy selected from the list consisting of anti-CTLA-4 monoclonal antibodies and/or anti-PD-L1 monoclonal antibodies.

11. The method of claim 9, wherein the immunotherapeutic agent is a cell-based therapy selected from the list consisting of dendritic-cell therapy and T-cell therapy.

12. The method of claim 7, wherein the additional therapeutic agent targets one of the receptors listed in Table 2.

13. The method of claim 7, wherein the additional therapeutic agent targets PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM and/or Sema7a.

14. The method of claim 1, comprising contacting the dysfunctional tumor antigen-specific CD8⁺ T cells with a therapeutic agent that targets one of the receptors listed in Table 2.

15. The method of claim 14, wherein the therapeutic agent targets PD-1, TIM-3, OX-40ICOS, TIGIT, CD244, TNFRSF18, Nrn1, Nrp1, KLRG1, GM156, GPNMB, GPR65, TMEM205, and TMEM126A, Nrn1, CRTAM and/or Sema7a.

16. The method of claim 15, wherein the therapeutic agent is an anti-Nrn1 antibody, antibody fragment, or antibody mimetic molecule.

17. The method of claim 15, wherein the therapeutic agent is an anti-Sema7a antibody, antibody fragment, or antibody mimetic molecule.

18. The method of claim 15, wherein the therapeutic agent is an anti-CRTAM antibody, antibody fragment, or antibody mimetic molecule.

19. A composition comprising: (a) one or more of an anti-4-1BB agent, an anti-LAG-3 agent, an anti-Nrn1 agent, an anti-Sema7a agent, and an anti-CRTAM agent; and (b) an immunotherapeutic agent, said composition formulated for therapeutic delivery to a subject.

20. The composition of claim 19, wherein the anti-4-1BB agent, anti-LAG-3 agent, anti-Nrn1 agent, anti-Sema7a agent, and/or anti-CRTAM agent is an antibody, antibody fragment, or antibody mimetic molecule.

21. A method comprising: (a) testing CD8⁺ T cells from a cell population to determine whether they co-express LAG-3 and 4-1BB; and (b) administering an anti-Nrn1 agent, an anti-Sema7a agent, and an anti-CRTAM agent.

22. The method of claim 21, wherein the anti-Nrn1 agent, anti-Sema7a agent, and/or anti-CRTAM agent is an antibody, antibody fragment, or antibody mimetic molecule.

23. The method of claim 21, wherein said testing is performed in vitro.

24. A method of identifying dysfunctional T cells by testing said cells for co-expression of 4-1BB and LAG-3.


SEQUENCE LISTING (60 sequences)

SEQ ID NO: 1 - DNA, 24 bp, synthetic construct
  source 1..24: mol_type = other DNA
ctcaaacaaggagaccttgggtgg

SEQ ID NO: 2 - DNA, 24 bp, synthetic construct
  source 1..24: mol_type = other DNA
cagacagctccaagctacttttac

SEQ ID NO: 3 - DNA, 24 bp, synthetic construct
  source 1..24: mol_type = other DNA
atgagccagggcagaaccttgtac

SEQ ID NO: 4 - DNA, 24 bp, synthetic construct
  source 1..24: mol_type = other DNA
gaaattcagtcctctgaggcagga

SEQ ID NO: 5 - DNA, 24 bp, synthetic construct
  source 1..24: mol_type = other DNA
ctaaagcctgatgactcggccaca

SEQ ID NO: 6 - DNA, 24 bp, synthetic construct
  source 1..24: mol_type = other DNA
ctttggagctagaggactctgccg

SEQ ID NO: 7 - DNA, 24 bp, synthetic construct
  source 1..24: mol_type = other DNA
ccttggaactggaggactctgcta

SEQ ID NO: 8 - DNA, 24 bp, synthetic construct
  source 1..24: mol_type = other DNA
gcccagaagaacgagatggccgtt

SEQ ID NO: 9 - DNA, 30 bp, synthetic construct
  source 1..30: mol_type = other DNA
ggattctgctaaaacaaaccagacatctgt

SEQ ID NO: 10 - DNA, 24 bp, synthetic construct
  source 1..24: mol_type = other DNA
gcttccctttctcagacagctgta

SEQ ID NO: 11 - DNA, 24 bp, synthetic construct
  source 1..24: mol_type = other DNA
gctaccccctctcagacatcagtg

SEQ ID NO: 12 - DNA, 23 bp, synthetic construct
  source 1..23: mol_type = other DNA
ggcttctccctctcagacatctt

SEQ ID NO: 13 - DNA, 24 bp, synthetic construct
  source 1..24: mol_type = other DNA
ctctctctacattggctctgcagg

SEQ ID NO: 14 - DNA, 24 bp, synthetic construct
  source 1..24: mol_type = other DNA
cttcgaatcaagtctgtagagccg

SEQ ID NO: 15 - DNA, 24 bp, synthetic construct
  source 1..24: mol_type = other DNA
tgaagatccagagcagcgggcccc

SEQ ID NO: 16 - DNA, 29 bp, synthetic construct
  source 1..29: mol_type = other DNA
ccactctgaagattcaacctacagaaccc

SEQ ID NO: 17 - DNA, 24 bp, synthetic construct
  source 1..24: mol_type = other DNA
caagatccagtctgcaaagcaggg

SEQ ID NO: 18 - DNA, 24 bp, synthetic construct
  source 1..24: mol_type = other DNA
gcacggagaagctgcttctcagcc

SEQ ID NO: 19 - DNA, 21 bp, synthetic construct
  source 1..21: mol_type = other DNA
gcatatcttgaagacagaggc

SEQ ID NO: 20 - DNA, 27 bp, synthetic construct
  source 1..27: mol_type = other DNA
ctctgaaaatccaacccacagcactgg

SEQ ID NO: 21 - DNA, 24 bp, synthetic construct
  source 1..24: mol_type = other DNA
tctgaagaagacgactcagcactg

SEQ ID NO: 22 - DNA, 24 bp, synthetic construct
  source 1..24: mol_type = other DNA
gcaaggcctggagacagcagtatc

SEQ ID NO: 23 - DNA, 19 bp, synthetic construct
  source 1..19: mol_type = other DNA
tgctttgggaagctccagt

SEQ ID NO: 24 - DNA, 20 bp, synthetic construct
  source 1..20: mol_type = other DNA
ccggtcttaagcacagacct

SEQ ID NO: 25 - DNA, 19 bp, synthetic construct
  source 1..19: mol_type = other DNA
ctacccggtggaagacctc

SEQ ID NO: 26 - DNA, 19 bp, synthetic construct
  source 1..19: mol_type = other DNA
tcaatcggctgcaagatgt

SEQ ID NO: 27 - DNA, 21 bp, synthetic construct
  source 1..21: mol_type = other DNA
agatccaacaacgaggagaca

SEQ ID NO: 28 - DNA, 19 bp, synthetic construct
  source 1..19: mol_type = other DNA
tcaccatgaaacccactgc

SEQ ID NO: 29 - DNA, 21 bp, synthetic construct
  source 1..21: mol_type = other DNA
caccctagcctaacctcaacc

SEQ ID NO: 30 - DNA, 20 bp, synthetic construct
  source 1..20: mol_type = other DNA
ctggcagatccagtcctgtt

SEQ ID NO: 31 - DNA, 20 bp, synthetic construct
  source 1..20: mol_type = other DNA
atgagggaggagagcggtat

SEQ ID NO: 32 - DNA, 20 bp, synthetic construct
  source 1..20: mol_type = other DNA
agaaaaccatcatgccaggt

SEQ ID NO: 33 - DNA, 18 bp, synthetic construct
  source 1..18: mol_type = other DNA
atcctcgcggtgcaaata

SEQ ID NO: 34 - DNA, 19 bp, synthetic construct
  source 1..19: mol_type = other DNA
ccggggagatctcatcaaa

SEQ ID NO: 35 - DNA, 24 bp, synthetic construct
  source 1..24: mol_type = other DNA
tgaagacacactacctgactcctg

SEQ ID NO: 36 - DNA, 21 bp, synthetic construct
  source 1..21: mol_type = other DNA
gctgctcactgtgaaggaagt

SEQ ID NO: 37 - DNA, 20 bp, synthetic construct
  source 1..20: mol_type = other DNA
caatctgtaccccgaggaga

SEQ ID NO: 38 - DNA, 19 bp, synthetic construct
  source 1..19: mol_type = other DNA
ctgtagcccacgtcgtagc

SEQ ID NO: 39 - DNA, 21 bp, synthetic construct
  source 1..21: mol_type = other DNA
gctgctcactgtgaaggaagt

SEQ ID NO: 40 - DNA, 19 bp, synthetic construct
  source 1..19: mol_type = other DNA
tcaccatgaaacccactgc

SEQ ID NO: 41 - DNA, 20 bp, synthetic construct
  source 1..20: mol_type = other DNA
tcttgctgtggcaattcaga

SEQ ID NO: 42 - DNA, 18 bp, synthetic construct
  source 1..18: mol_type = other DNA
gctgcagggaagatggac

SEQ ID NO: 43 - DNA, 20 bp, synthetic construct
  source 1..20: mol_type = other DNA
gaacggtactggcgtctgtc

SEQ ID NO: 44 - DNA, 21 bp, synthetic construct
  source 1..21: mol_type = other DNA
aatgttgatcatgccatctcc

SEQ ID NO: 45 - DNA, 21 bp, synthetic construct
  source 1..21: mol_type = other DNA
cgcagacagctgagtagttcc

SEQ ID NO: 46 - DNA, 20 bp, synthetic construct
  source 1..20: mol_type = other DNA
tcatgcaacgcttagactgg

SEQ ID NO: 47 - DNA, 20 bp, synthetic construct
  source 1..20: mol_type = other DNA
agcagcagctattggagacc

SEQ ID NO: 48 - DNA, 20 bp, synthetic construct
  source 1..20: mol_type = other DNA
tgaaaacctcctcccctctt

SEQ ID NO: 49 - DNA, 19 bp, synthetic construct
  source 1..19: mol_type = other DNA
acccagttcatgccatcct

SEQ ID NO: 50 - DNA, 21 bp, synthetic construct
  source 1..21: mol_type = other DNA
cagcttgttgtccaaatcgtc

SEQ ID NO: 51 - DNA, 20 bp, synthetic construct
  source 1..20: mol_type = other DNA
acaggtgaagcaggtctcgt

SEQ ID NO: 52 - DNA, 21 bp, synthetic construct
  source 1..21: mol_type = other DNA
gcccttaaagactgcatcaca

SEQ ID NO: 53 - DNA, 19 bp, synthetic construct
  source 1..19: mol_type = other DNA
tcgaaggccatgtcatctg

SEQ ID NO: 54 - DNA, 20 bp, synthetic construct
  source 1..20: mol_type = other DNA
cccacaatgtgttgcagttc

SEQ ID NO: 55 - DNA, 20 bp, synthetic construct
  source 1..20: mol_type = other DNA
tggggaatgcattttaccat

SEQ ID NO: 56 - DNA, 21 bp, synthetic construct
  source 1..21: mol_type = other DNA
ccgatgtccatcacattctct

SEQ ID NO: 57 - DNA, 18 bp, synthetic construct
  source 1..18: mol_type = other DNA
ttgagatccatgccgttg

SEQ ID NO: 58 - DNA, 20 bp, synthetic construct
  source 1..20: mol_type = other DNA
tggggaatgcattttaccat

SEQ ID NO: 59 - DNA, 20 bp, synthetic construct
  source 1..20: mol_type = other DNA
agcagcagctattggagacc

SEQ ID NO: 60 - DNA, 20 bp, synthetic construct
  source 1..20: mol_type = other DNA
gcagagggtgacggatgtag